(19) United States
(12) Patent Application Publication
(10) Pub. No.: US 2023/0137764 A1
(43) Pub. Date: May 4, 2023

(54) PRODRUGS OF FULVESTRANT

(71) Applicant: KASHIV BIOSCIENCES, LLC, Piscataway, NJ (US)

(72) Inventors: Parva Yogeshchandra PUROHIT, Ahmedabad (IN); Pathik Subhashchandra BRAHMKSHATRIYA, Ahmedabad (IN); Vishalgiri Gunvantgiri GOSWAMI, Ahmedabad (IN)

(21) Appl. No.: 17/779,015

(22) PCT Filed: Nov. 24, 2020
(86) PCT No.: PCT/IB2020/061065

§ 371 Date: May 23, 2022

(30) Foreign Application Priority Data
Nov. 24, 2019 (IN) 201921047986
Jul. 8, 2020 (IN) 202021029084

(51) Int. Cl. C07J 43/00; C07J 31/00; C07J 41/00

(52) U.S. Cl. CPC C07J 43/003; C07J 31/006; C07J 41/0072; C07J 43/006

(57) ABSTRACT

The present invention relates to Fulvestrant prodrugs and process for the preparation thereof. The present disclosure also relates to pharmaceutical composition of Fulvestrant prodrugs and method of treatment using the same.

### FIELD OF INVENTION

The present invention relates to Fulvestrant prodrugs and process for the preparation thereof. The present disclosure also relates to pharmaceutical composition of Fulvestrant prodrugs and method of treatment using the same.

### BACKGROUND OF THE INVENTION

Fulvestrant is an estrogen receptor antagonist marketed as Faslodex™ for the treatment of hormone receptor positive metastatic breast cancer in postmenopausal women with disease progression following anti-estrogen therapy. Fulvestrant has clinical therapeutic effect in patients failed in treatment with tamoxifen. Therefore, among many drugs for treating breast cancer, Fulvestrant is the only anti-estrogen agent that may be widely used in clinical treatment after the failure of tamoxifen, which has initiated a new way of treating hormone-sensitive breast cancer. Due to the poor solubility and oral bioavailability of Fulvestrant, the drug is currently administered via intramuscular injection of an oil-based Fulvestrant formulation. The Faslodex™ product is approved for administration by intramuscular injection on days 1, 15, 29 and once monthly thereafter. This injection contains upto 10% of benzyl alcohol which might act as anaesthetic level while castor oil is used as release rate modifier which can be painful at the time of injection due to high viscosity. According to FDA drug approval summaries, injection site reaction and hot flashes were observed.

Fulvestrant is a highly lipophilic molecule which is practically insoluble in water. This restricts their bioavailability. A drug with poor solubility will often exhibit poor bioavailability and require administration of high dosages to attain therapeutically effective blood levels of the drug.

U.S. Pat. No. 6,774,122 B2 describes that intra-muscular injections of Fulvestrant in the form of an aqueous suspension were not suitable for use. Those suspensions resulted in extensive local tissue irritation at the injection site as well as a poor release profile due to the presence of Fulvestrant in the form of solid particles.

The use of prodrugs allows the artisan to modify one or more properties of a biologically active compound. Prodrugs include chemical derivatives of a biologically active parent compound which, upon administration, will eventually liberate the active parent compound in vivo. The rate of release of the active drug is influenced by several factors including the rate of hydrolysis of the linker which joins the parent biologically active compound to the prodrug carrier.

WO2016/004166 A1 discloses boron based Fulvestrant prodrugs for the treatment of breast cancer. The application also discloses the need of improved bioavailability of Fulvestrant to make it more effective therapeutic regimen for tamoxifen-resistant breast cancer. Despite clinical efficacy of Fulvestrant, the utility of Fulvestrant has been limited by the amount of drug that can be administered in a single injection and by reduced bioavailability.

J. Med. Chem., 2016, 59 (17), pp. 8134-8140 discloses that Fulvestrant undergoes rapid and extensive O-glucoronidation and O-sulfation to form polar phase II metabolites that are inactive and water soluble.

Accordingly, an object of the present invention is to provide a compound or a salt thereof that can overcome the above challenges and is metabolized rapidly to produce Fulvestrant in the body.

### SUMMARY OF THE INVENTION

In one embodiment, the present invention relates to compound of formula I:

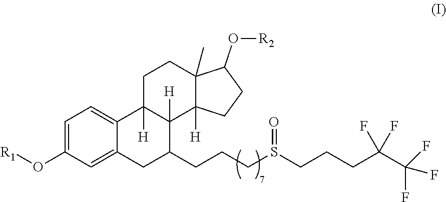

wherein; R₁ is

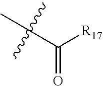

wherein R₁₇ is selected from NH₂, NHR₁₈, or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and;
R₂ is selected independently from group comprising of:
i)

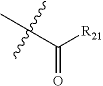

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl;
ii)

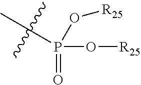

wherein each R₂₅ is selected independently from hydrogen; optionally substituted alkyl, aryl, or heteroaryl;
iii)

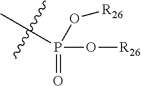

wherein each R₂₆ is selected independently from hydrogen; optionally substituted alkyl, cycloalkyl, heterocycloalkyl, aryl, or heteroaryl;
iv) amino acids, wherein the amino acid is linked via ester linkage at the point of attachment; and
enantiomers, diastereomers, racemates, pharmaceutically acceptable salts or solvates thereof.

In another embodiment, the present invention relates to compound of formula IV:

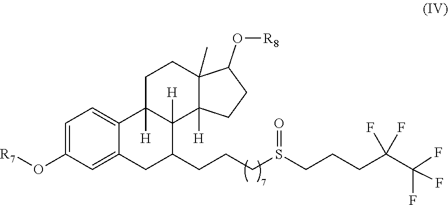

wherein R₇ is

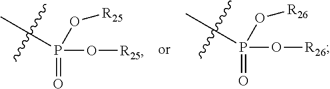

wherein each R₂₅ and R₂₆ is selected independently from hydrogen; optionally substituted alkyl, aryl or heteroaryl; and
R₈ is selected independently from group comprising of:
i)

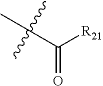

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl;
ii)

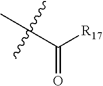

wherein R₁₇ is selected from NH₂, NHR₁₈ or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, —C(O)-aminoacid, carboxyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
iii)

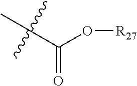

wherein R₂₇ is selected from group comprising optionally substituted alkyl, alkenyl, alkynyl, cycloalkyl, heterocycloalkyl, aryl, or heteroaryl;
iv)

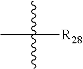

wherein R₂₈ is selected from group comprising optionally substituted alkyl, alkenyl, alkynyl, alkoxy, cycloalkyl, heterocycloalkyl, aryl or heteroaryl; wherein one or more substitution is selected from formyl, halo, phosphate, cyano, nitro, or amino;
v) amino acid, wherein the amino acid is linked via ester linkage at the point of attachment; and
enantiomers, diastereomers, racemates, pharmaceutically acceptable salts or solvates thereof.

In another embodiment, the present invention relates to compound of formula VI:

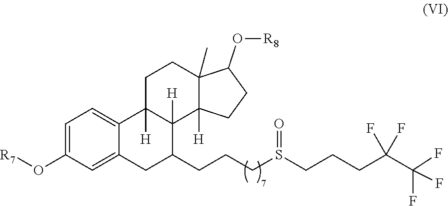

wherein, R₁₀ is

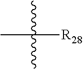

wherein R₂₈ is selected from group comprising optionally substituted alkyl, alkenyl, alkynyl, alkoxy, cycloalkyl, heterocycloalkyl, aryl, or heteroaryl; wherein one or more substitution is selected from formyl, halo, phosphate, cyano, nitro, amino, hydroxy, heterocycloalkyl, or alkoxy; and
R₁₁ is hydrogen or

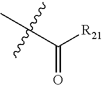

where R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
with the proviso that when R₁₁ is hydrogen, R₁₀ is not methyl or ethyl; and
enantiomers, diastereomers, racemates, pharmaceutically acceptable salts or solvates thereof.

In another embodiment, the present invention relates to compound of formula VIII:

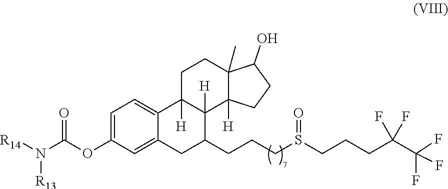

wherein R₁₃ is selected from hydrogen, alkyl; and
R₁₄ is selected from group comprising optionally substituted alkyl, heterocycloalkyl, or aryl; wherein one or more substitution is selected from alkyl, alkoxy, halo, heteroaryl, heterocycloalkyl, cycloalkyl, amino, or hydroxy; and
enantiomers, diastereomers, racemates, pharmaceutically acceptable salts or solvates thereof.
   In another embodiment, the present invention relates to prodrugs of Fulvestrant of formula IX:

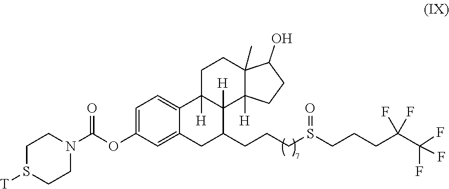

wherein S is selected from O, C, or N; and
T is selected from group comprising optionally substituted aryl, heteroaryl, heterocycloalkyl, cycloalkyl, amino,

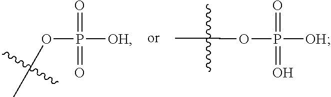

wherein one or more substitution is selected from alkyl, alkoxy; and enantiomers, diastereomers, racemates, pharmaceutically acceptable salts or solvates thereof.

In another embodiment, the present invention relates to prodrugs of Fulvestrant of formula X:

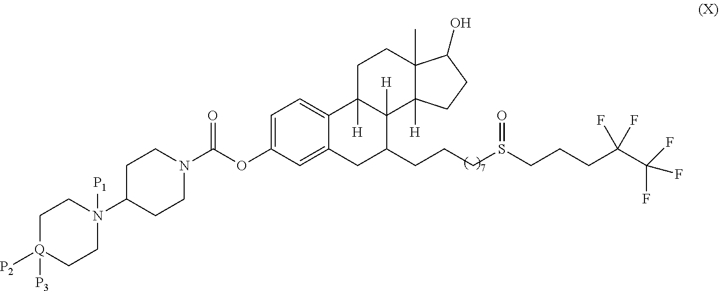

wherein, Q is selected from C or N; and
when Q is C, P₁ is selected independently from optionally substituted alkyl, optionally substituted

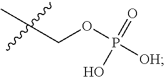 [SMILES: *COP(=O)(O)O]

wherein substitution on

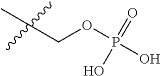 [SMILES: *COP(=O)(O)O]

is alkyl; and P₂ and P₃ is selected independently from hydrogen or alkyl; and
when Q is N, P₂ is selected independently from optionally substituted alkyl, optionally substituted

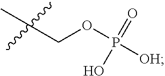 [SMILES: *COP(=O)(O)O]

wherein substitution on

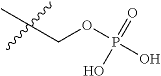 [SMILES: *COP(=O)(O)O]

is alkyl; P₁ is hydrogen and P₃ is optionally substituted alkyl; and
enantiomers, diastereomers, racemates, pharmaceutically acceptable salts or solvates thereof.

In another embodiment, the present invention relates to compound of formula XI:

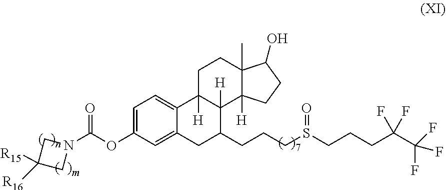

wherein R₁₅ and R₁₆ together forms a cyclic structure; wherein the said cyclic structure is selected from optionally substituted cycloalkyl, or heterocycloalkyl; wherein one or more substitution is selected from alkyl, alkoxy, halo, amino, or hydroxy; m and n can be independently 1 to 3; and enantiomers, diastereomers, racemates, pharmaceutically acceptable salts or solvates thereof.

In a preferred embodiment, the present invention relates to compound of formula XI where m and n both are 1 or 2.

In another embodiment, the present invention relates to pharmaceutical composition comprising Fulvestrant prodrug and enantiomers, diastereomers, racemates, pharmaceutically acceptable salts and solvates thereof and pharmaceutically acceptable excipients.

The pharmaceutical compositions of the present disclosure can be in any form known to those of skill in the art. The pharmaceutical compositions of this invention may be administered orally, parenterally, by inhalation spray, topically, rectally, nasally, buccally or via an implanted reservoir, preferably oral administration or administration by injection.

In another embodiment, the invention provides use of compounds of the present invention in the preparation of a pharmaceutical formulation as describe hereinabove, for the treatment of a benign or malignant disease of the breast or reproductive tract, preferably treating breast cancer.

In another embodiment, the invention provides compounds for the treatment of a benign or malignant disease of the breast or reproductive tract, preferably treating breast cancer.

### DETAILED DESCRIPTION OF THE INVENTION

As used herein, the term “prodrug” refers to a precursor compound that, following administration, releases a biologically active compound in vivo via a chemical or physiological process. A prodrug itself may either lack or possess the desired biological activity.

The terms “a” and “an” do not denote a limitation of quantity, but rather denote the presence of at least one of the referenced items.

The term “about” as used herein, when referring to a measurable value is meant to encompass variations of ±10%, preferably ±5%, more preferably ±1% and still more preferably ±0.1% from the specified value.

The term “cancer” refers to conditions including solid cancers, lymphomas and leukemias. Examples of different types of cancer include, but are not limited to, breast cancer, lung cancer, ovarian cancer, prostate cancer, colorectal cancer, liver cancer, renal cancer, bladder cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach cancer, cancer of the central nervous system, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, small cell lymphoma, large cell lymphoma, monocytic leukemia, myelogenous leukemia, acute lymphocytic leukemia, acute myelocytic leukemia, and multiple myeloma.

As used herein, the term “alkyl” refers to a straight or branched, saturated, aliphatic radical having from 1 to about 10 carbon atoms., for example, methyl, ethyl, propyl, isopropyl, n-butyl, i-butyl, t-butyl and the like.

The term “alkenyl” refers to a straight chain or branched hydrocarbon having at least 2 carbon atoms and at least one carbon-carbon double bond. Alkenyl groups can have any suitable number of double bonds, including, but not limited to 1, 2, 3, 4, 5 or more. Preferable alkenyl groups include ethenyl (—CH═CH₂), 2-propenyl (allyl, —CH2-CH═CH₂) and the like.

The term “alkynyl” denotes an alkynyl groups having from 2 to 10 carbon atoms and having at least 1-2 sites of alkynyl unsaturation, preferred alkynyl groups include ethynyl (—C≡CH), propargyl (—CH₂—C≡CH), 1-butenyl, 2-butenyl, isobutenyl, butadienyl and the like.

The term “cycloalkyl” denotes a saturated carbocyclic group having from 3 to 10 carbon atoms having a single ring (e.g., cyclohexyl) or multiple condensed rings (e.g., norbornyl). Preferred cycloalkyl include cyclopropyl, cyclobutyl, cyclopentyl, cyclohexyl, cycloheptyl, norbornyl and the like.

The term “heterocyclic ring” denotes organic compounds that contain a ring structure containing atoms in addition to carbon, such as sulphur, oxygen or nitrogen, as part of the ring. There can be more than one hetero atom substitution in the ring structure selected from sulphur, oxygen or nitrogen. The ring can be saturated, partially saturated or unsaturated ring structure which includes “heterocycloalkyl” and “heteroaryl”.

The term “heterocycloalkyl” denotes a C₃-C₁₀ cycloalkyl group according to the definition above, in which up to 4 carbon atoms are replaced by heteroatoms chosen from the group consisting of O, S or N. Preferred heterocycloalkyl include pyrrolidine, piperidine, piperazine, morpholine, tetrahydrofuran, tetrahydrothiophenyl, and the like.

The term “aryl” denotes a cyclic aromatic hydrocarbon radical consisting of one or more fused rings containing 6-14 carbon atoms in which at least one ring is aromatic in nature, for example phenyl, naphthyl, 1,2,3,4-tetrahydronaphthalenyl, indanyl and the like.

The term “heteroaryl” denotes a cyclic aromatic hydrocarbon radical consisting of one or more fused rings containing 5-14 ring atoms, preferably containing 5-10 ring atoms, in which at least one ring is aromatic in nature, and which contains at least one heteroatom, selected from N, O or S, for example pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, benzothienyl, benzotriazolyl, isobenzothienyl, indolyl, isoindolyl, benzimidazolyl, imidazo[1,2-a]pyridyl, benzothiazolyl, benzoxazolyl, quinolizinyl, quinazolinyl, benzoquinolyl and the like.

The term “halogen” denotes chlorine, iodine, fluorine and bromine.

As used herein, the term “leaving group” or “L” or “L₁” can be defined as part of a substrate that cleaved by the action of a nucleophile. Examples of leaving groups include, but are not limited to: halogen (F, CI, Br, and I), tosylate, mesylate, triflate, acetate, hydroxyl, camphorsulfonate, aryloxide, and aryloxide and the like.

The term “alkoxy” refers to the group —O-alkyl.

The term “alkoxyalkyloxy” refers to the group alkyl-O-alky-O—

The term “alkoxycarbonyloxy” refers to the group alkyl-O—CO—O—.

The term “phosphate” refers to —O—PO(OH)₂.

The term “phosphonyl” refers to —PO₃H₂.

The term “alkylphosphate” refers to (-alkyl-O—PO(OH)₂).

The term amino acid as used herein refers to two stereoisomeric forms, called “D” and “L.” The D and L form of any amino acid have identical physical properties and chemical reactivities, but rotate the plane of plane-polarized light equally but in opposite directions and react at different rates with asymmetric reagents. All naturally occurring amino acids in proteins are in the L form. Amino acid comprises lysine, valine, tryptophan, phenylalanine, methionine, leucine, threonine, isoleucine, arginine, histidine, tyrosine, carnitine, serine, glutamine, aspartic acid, proline, glycine, cysteine, alanine, glutamic acid. Amino acid may be present as either “D” or “L” enantiomer.

The term “—C(O)-aminoacid” refers to the amino acid attached to carbonyl group via amide or ester linkage: more preferably via amide linkage.

The term “optionally” means the subsequently described event or circumstance can or cannot occur, and that the description includes instances where the event or circumstance occurs and instances where it does not.

The term “pharmaceutically acceptable carrier” refers to a non-toxic carrier that may be administered to a patient, together with a compound of this invention, and which does not destroy the pharmacological activity thereof.

The term “pharmaceutically acceptable excipient” as used herein includes vehicles, adjuvants, or diluents or other auxiliary substances, such as those conventional in the art, which are readily available to the public. For example, pharmaceutically acceptable excipients include pH adjusting and buffering agents, tonicity adjusting agents, stabilizers, wetting agents and the like.

As used herein, the term “salt” refers to an acid or base salt of a compound of the invention. Salts of basic compounds are salts formed with mineral acids, organic carboxylic acids, organic sulfonic acids, and the like. Examples of suitable acids include hydrochloric, hydrobromic, sulfuric, nitric, perchloric, fumaric, maleic, phosphoric, glycollic, lactic, salicylic, succinic, toluene-p-sulfonic, tartaric, acetic, citric, methanesulfonic, formic, benzoic, malonic, naphthalene-2-sulfonic and benzenesulfonic acids. Salts of acidic compounds are formed with bases, namely cationic species such as alkali and alkaline earth metal cations e.g., sodium, lithium, potassium, calcium, and magnesium ions as well as ammonium cations e.g., ammonium, trimethylammonium and diethylammonium. Salts of acidic compounds are formed with organic bases, namely tromethamine and meglumine.

The compounds of this invention contain one or more asymmetric carbon atoms and thus occur as racemate and racemic mixtures, single enantiomers, diastereomeric mixtures and individual diastereomers. All such isomeric forms of these compounds are expressly included in the present invention. Each stereogenic carbon may be of the R or S configuration.

As used herein, the term “deprotecting agents” includes, but are not limited to, hydrogen and palladium on carbon (H₂, Pd/C), ammonium formate and palladium on carbon (HCOONH₄, Pd/C), hydrogen and palladium hydroxide on carbon (H₂, Pd(OH)₂/C), combination of Pd/C and Pd(OH)₂/C and acid such as hydrochloride acid, hydrobromic acid and the like.

For purposes of the present invention, the term “substituted” shall be understood to include adding or replacing one or more atoms contained within a functional group or compound with one or more different atoms.

Unless otherwise constrained by the definition of the individual substituent, the above set out groups, like “alkyl”, “alkenyl”, “alkynyl”, “cycloalkyl”, “heterocycloalkyl”, “aryl” and “heteroaryl” etc. groups can optionally be substituted with one or more substituents selected from the group consisting of “C1-C8-alkyl”, “C2-C8-alkenyl”, “C2-C8-alkynyl”, “cycloalkyl”, “heterocycloalkyl”, “aryl”, “heteroaryl”, “amino”, “alkylamino”, “acyl”, “acyloxy”, “acylamino”, “aminocarbonyl”, “alkoxycarbonyl”, “alkoxyalkyloxy”, “alkoxycarbonyloxy”, “carbamate,” “sulfinyl”, “sulfonyl”, “alkoxy”, “sulfanyl”, “halogen”, “carboxy”, “trihalomethyl”, “cyano”, “hydroxy”, “mercapto”, “nitro”, “phosphate”, “alkylphosphate” and the like. The preferred one or more substituents is selected from 1 to 10 in number; more preferably from 1 to 5 in number.

The term “coupling reagent” as used herein includes but not limited to O-benzotriazole-N,N,N′,N′-tetramethyl-uronium-hexafluoro-phosphate (HBTU), 2-(7-Aza-1H-benzotriazole-1-yl)-1,1,3,3-tetramethyluroniumhexafluorophosphate (HATU), acid halide, 1-hydroxybenzotriazole (HOBt), 1-Hydroxy-7-aza-1H-benzotriazole (HOAt), diisopropylcarbodiimide (DIC), dicyclohexylcarbodiimide (DCC), N-(3-Dimethylaminopropyl)-N′-ethylcarbodiimide hydrochloride (EDC), 2-(6-Chloro-1H-benzotriazol-1-yl)-N,N,N′,N′-tetramethylaminium hexafluoro-phosphate (HCTU), 1-[1-(Cyano-2-ethoxy-2-oxoethylideneaminooxy) dimethyl-aminomorpholino]-uroniumhexa-fluorophosphate (COMU) and the like.

Base used in the present invention can be inorganic and organic base. The examples of organic base includes but not limiting to amines such as diisopropylethylamine, triethylamine, pyridine, 1,8-diazabicyclo[5.4.0]undec-7-ene (DBU), imidazole, N,N-dimethyl aniline, N,N-dimethyl amino pyridine (DMAP), 1,5-diazabicyclo[4.3.0]non-5-ene (DBN) and the like or mixtures thereof. The examples of inorganic base includes but not limiting to alkali or alkaline earth metal carbonate, bicarbonate, hydroxide or phosphate such as potassium carbonate, sodium carbonate, lithium carbonate, sodium bicarbonate, potassium bicarbonate, lithium bicarbonate, sodium hydroxide, potassium hydroxide, lithium hydroxide, potassium phosphate, sodium phosphate; hydride such as sodium hydride, lithium hydride or potassium hydride; alkoxide such as sodium or potassium methoxide or ethoxide, tertiary butoxide and the like or mixtures thereof.

As used herein, the term “solvent” refers to the solvents include, but are not limited to, nitriles such as acetonitrile, propionitrile, butyronitrile, isobutyronitrile and the like; ethers such as dioxane, diethyl ether, diisopropylether, tetrahydrofuran, dimethoxyethane and the like; hydrocarbon such as toluene, xylene, hexane, heptane, cyclohexane and the like; chlorinated hydrocarbon such as methylene chloride, ethylene dichloride, carbon tetra chloride, chloroform, chlorobenzene and the like; polar aprotic solvents such as N,N-dimethylformamide (DMF), dimethyl acetamide (DMAc), dimethyl sulfoxide (DMSO) and the like or mixtures thereof.

The novel compounds of the present invention can be used in conventional solid or liquid pharmaceutical forms, for example as uncoated or film coated tablets, capsules, powders, granules, solutions or sprays. The active substances can for this purpose be processed with conventional pharmaceutical aids such as tablet binders, bulking agents, preservatives, tablet disintegrants, flow regulators, plasticizers, wetting agents, dispersants, emulsifiers, solvents, release-slowing agents, antioxidants and/or propellant gases.

The prodrugs of the present invention are characterized by unexpectedly high aqueous solubility. This solubility facilitates administration of higher doses of the prodrug, resulting in a greater drug load per unit dosage. The prodrugs of the present invention are also characterized by facile hydrolytic cleavage to release the active Fulvestrant in vivo. The high aqueous solubility and the facile in vivo metabolism result in a greater bioavailability of the drug.

When the prodrugs of this invention are administered in combination therapies with other agents, they may be administered sequentially or concurrently to the patient. Alternatively, pharmaceutical compositions according to this invention may be comprised of a combination of a prodrug of this invention and another therapeutic agent.

In one embodiment, the present invention relates to compound of formula I:

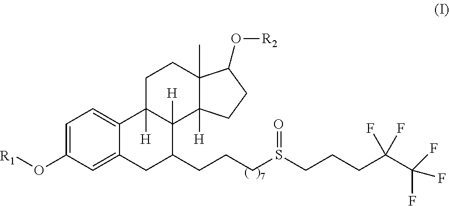

wherein; R₁ is

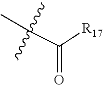

wherein R₁₇ is selected from NH₂, NHR₁₈, or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and;
R₂ is selected independently from group comprising of:
i)

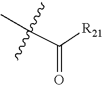

wherein R₂₁ is selected from group comprising optionally substituted alkyl, alkenyl, alkynyl, cycloalkyl, aryl, heteroaryl; or —CR₂₂R₂₃, where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl;
ii)

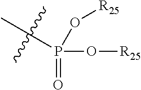

wherein each R₂₅ is selected independently from hydrogen; optionally substituted alkyl, aryl, or heteroaryl;
iii)

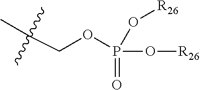

wherein each R₂₆ is selected independently from hydrogen; optionally substituted alkyl, cycloalkyl, heterocycloalkyl, aryl, or heteroaryl;
iv) amino acids, wherein the amino acid is linked via ester linkage at the point of attachment; and
enantiomers, diastereomers, racemates, pharmaceutically acceptable salts or solvates thereof.

In one embodiment, the present invention relates to compound of formula I:

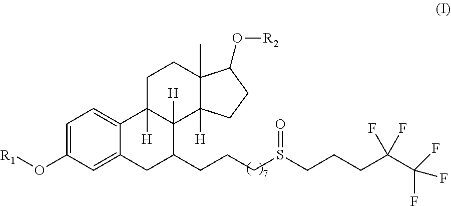

wherein; R₁ is

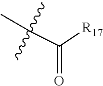

wherein R₁₇ is selected from NH₂, NHR₁₈, or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals,
wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and;
R₂ is

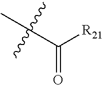

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl; and enantiomers, diastereomers, racemates, pharmaceutically acceptable salts or solvates thereof.

In one embodiment, the present invention relates to compound of formula I:

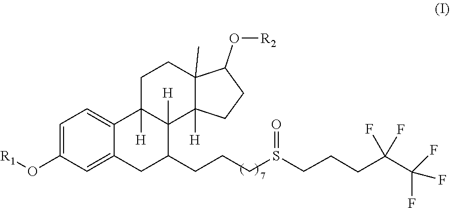

wherein; R₁ is

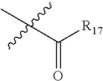

wherein R₁₇ is selected from NH₂, NHR₁₈, or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals,
wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and;
R₂ is

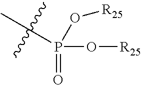

wherein each R₂₅ is selected independently from hydrogen; optionally substituted alkyl, aryl, or heteroaryl; and enantiomers, diastereomers, racemates, pharmaceutically acceptable salts or solvates thereof.

In one embodiment, the present invention relates to compound of formula I:

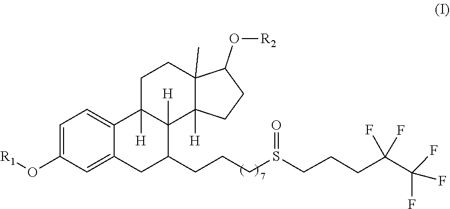

wherein; R₁ is

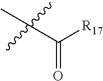

wherein R₁₇ is selected from NH₂, NHR₁₈, or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals,
wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and;
R₂ is

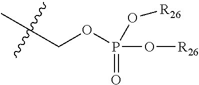

wherein each R₂₆ is selected independently from hydrogen; optionally substituted alkyl, cycloalkyl, heterocycloalkyl, aryl, or heteroaryl; and enantiomers, diastereomers, racemates, pharmaceutically acceptable salts or solvates thereof.

In one embodiment, the present invention relates to compound of formula I:

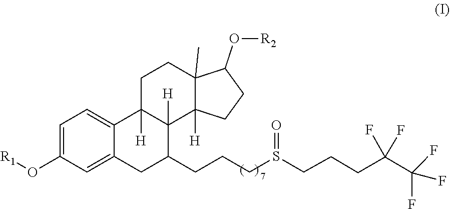

wherein; R₁ is

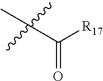

wherein R₁₇ is selected from NH₂, NHR₁₈, or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals,
wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and;
R₂ is amino acids, wherein the amino acid is linked via ester linkage at the point of attachment;
and enantiomers, diastereomers, racemates, pharmaceutically acceptable salts or solvates thereof.

In another embodiment, the present invention relates to compound of formula II:

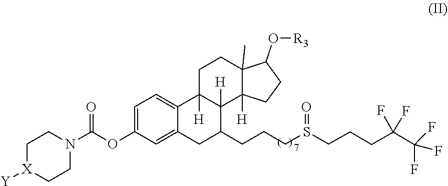

wherein X is selected from O, C, N; and
Y is selected from optionally substituted aryl, heteroaryl, heterocycloalkyl, or cycloalkyl; and
R₃ is selected independently from group comprising of:
i)

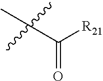

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, alkoxycarbonyl, alkenyl, alkynyl, alkylthio and arylcarbonyl;
ii)

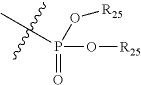

wherein each R₂₅ is selected independently from hydrogen; optionally substituted alkyl, aryl, or heteroaryl;
iii)

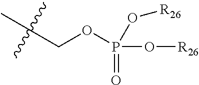

wherein each R₂₆ is selected independently from hydrogen; optionally substituted alkyl, cycloalkyl, heterocycloalkyl, aryl, or heteroaryl;
iv) amino acids, wherein the amino acid is linked via ester linkage at the point of attachment; and
enantiomers, diastereomers, racemates, pharmaceutically acceptable salts or solvates thereof.

In preferred embodiment, the present invention relates to compound of formula II:

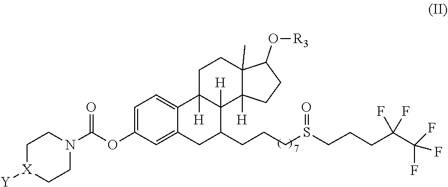

wherein X is selected from O, C, N; and
Y is selected from group comprising optionally substituted aryl, heteroaryl, heterocycloalkyl, or cycloalkyl; wherein the one or more substitution is selected from alkyl or alkoxy; and
R₃ is selected independently from group comprising of:
i)

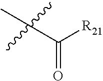

wherein R₂₁ is optionally substituted alkyl; wherein one or more substitution is selected from amino, phosphate, alkylphosphate, or hydroxy;
ii)

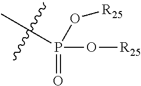

wherein each R₂₅ is selected independently from group comprising of hydrogen; optionally substituted alkyl;
iii)

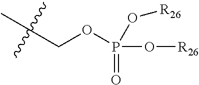

wherein each R₂₆ is selected independently from hydrogen; optionally substituted alkyl;
iv) amino acids; wherein the amino acid is linked via ester linkage at the point of attachment; and
enantiomers, diastereomers, racemates, pharmaceutically acceptable salts or solvates thereof.

In another embodiment, the present invention relates to compound of formula III:

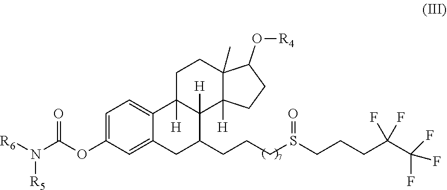

wherein; R₅ is selected from hydrogen, alkyl;
R₆ is selected from group comprising optionally substituted alkyl, aryl, heteroaryl, heterocycloalkyl, or cycloalkyl; and
R₄ is selected independently from group comprising of:
i)

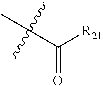

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, hydroxy, alkylcarbonyl, formyl, halo, alkylphosphate, phosphate, cyano, nitro, alkoxy, amino, alkoxycarbonyl, alkenyl, alkynyl, alkylthio and arylcarbonyl;
ii)

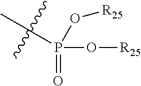

wherein each R₂₅ is selected independently from hydrogen; optionally substituted alkyl, aryl, or heteroaryl;
iii)

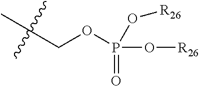

wherein each R₂₆ is selected independently from hydrogen; optionally substituted alkyl, cycloalkyl, heterocycloalkyl, aryl, or heteroaryl;
iv) amino acids, wherein the amino acid is linked via ester linkage at the point of attachment; and
enantiomers, diastereomers, racemates, pharmaceutically acceptable salts or solvates thereof.

In another embodiment, the present invention relates compound of formula III:

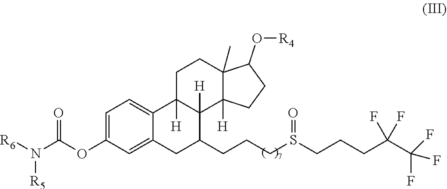

Wherein R₅ is selected from hydrogen, alkyl;
R₆ is selected from group comprising optionally substituted alkyl or aryl, wherein one or more substitution is selected from alkyl, alkoxy, halo, heteroaryl, heterocycloalkyl, cycloalkyl, amino, or hydroxy; and
R₄ is selected independently from group consisting of:
i)

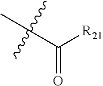

wherein R₂₁ is optionally substituted alkyl; wherein one or more substitution is selected from amino, phosphate, alkylphosphate, or hydroxy;
ii)

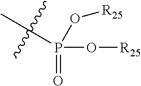

wherein each R₂₅ is selected independently from hydrogen, or optionally substituted alkyl;
iii)

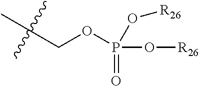

wherein each R₂₆ is selected independently from hydrogen or optionally substituted alkyl;
iv) amino acids, wherein the amino acid is linked via ester linkage at the point of attachment; and
enantiomers, diastereomers, racemates, pharmaceutically acceptable salts or solvates thereof.

In another embodiment, the present invention relates to compound selected from group consisting of:

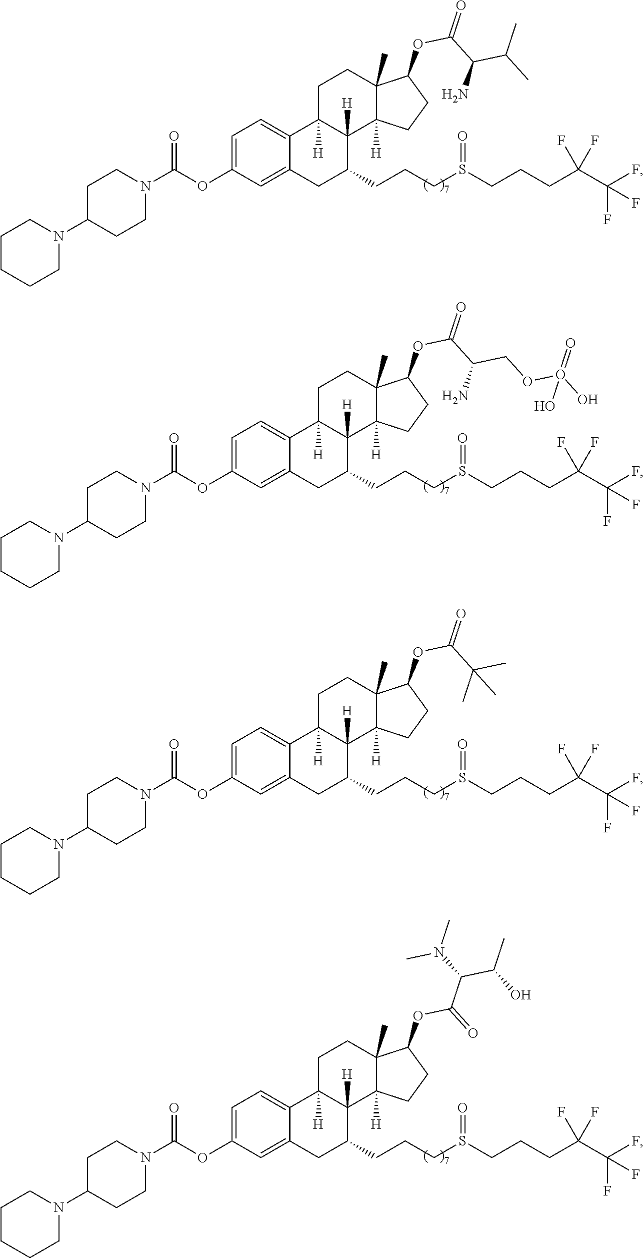

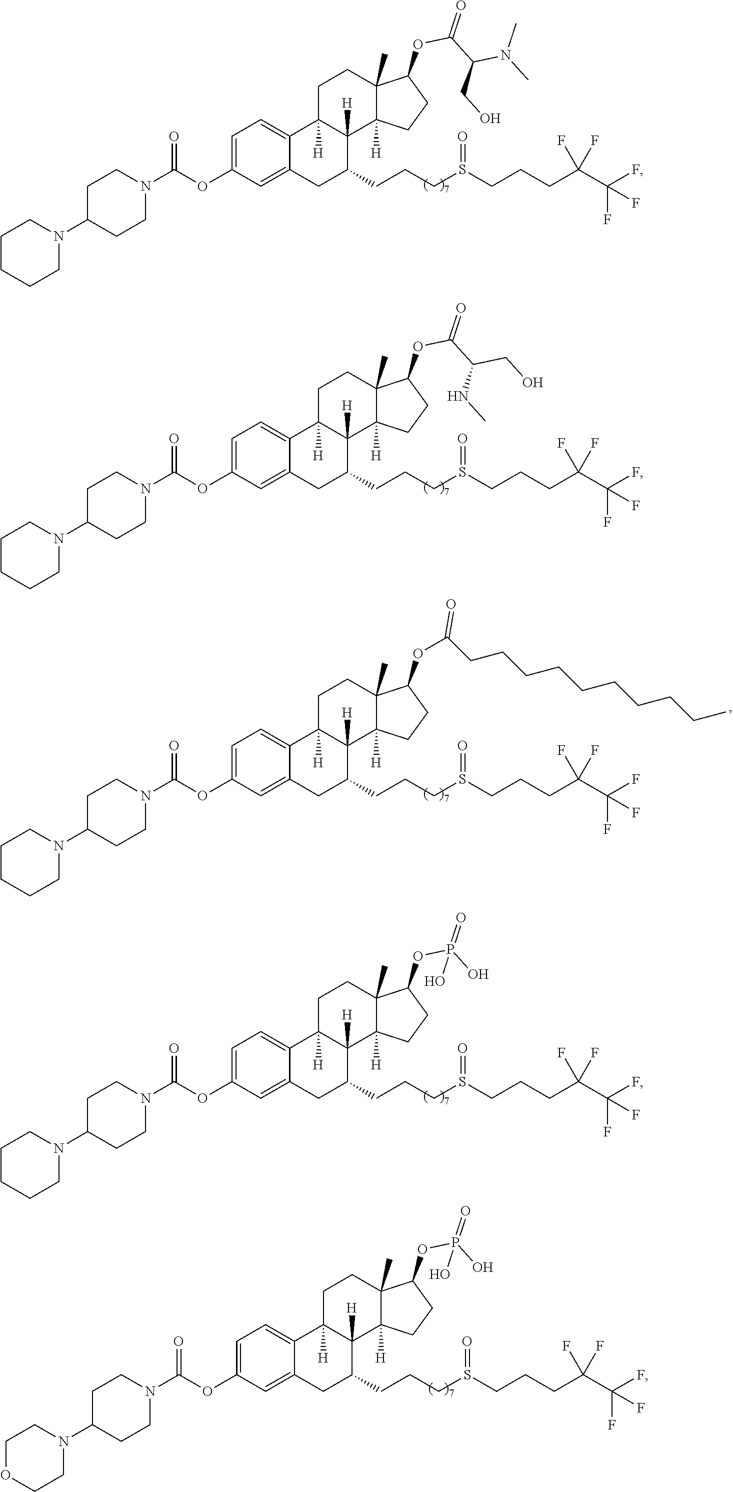

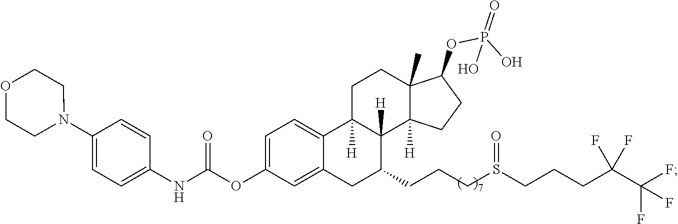

and pharmaceutically acceptable salts or solvates thereof.

In another embodiment, the present invention relates to compound of formula IV:

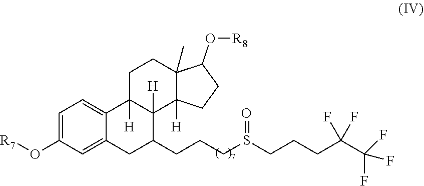

wherein R₇ is

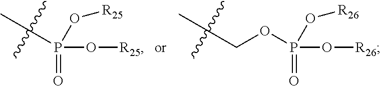

wherein each R₂₅ and R₂₆ is selected independently from hydrogen; optionally substituted alkyl, aryl or heteroaryl; and
R₈ is selected independently from group comprising of:
i)

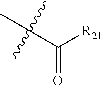

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl;
ii)

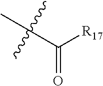

wherein R₁₇ is selected from NH₂, NHR₁₈ or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, —C(O)-aminoacid, carboxyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
iii)

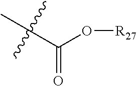

wherein R₂₇ is selected from group comprising optionally substituted alkyl, alkenyl, alkynyl, cycloalkyl, heterocycloalkyl, aryl, or heteroaryl;
iv)

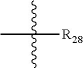

wherein R₂₈ is selected from group comprising optionally substituted alkyl, alkenyl, alkynyl, alkoxy, cycloalkyl, heterocycloalkyl, aryl or heteroaryl; wherein one or more substitution is selected from formyl, halo, phosphate, cyano, nitro, or amino;
v) amino acid, wherein the amino acid is linked via ester linkage at the point of attachment; and
enantiomers, diastereomers, racemates, pharmaceutically acceptable salts or solvates thereof.

In another embodiment, the present invention relates to compound of formula IV:

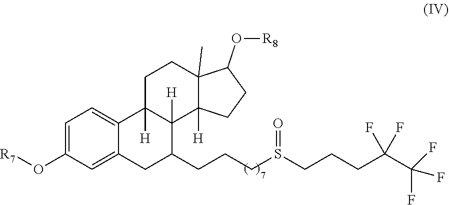

wherein R₇ is

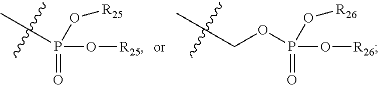

wherein each R₂₅ and R₂₆ is selected independently from hydrogen; optionally substituted alkyl, aryl or heteroaryl; and
R₈ is

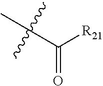

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl; and enantiomers, diastereomers, racemates, pharmaceutically acceptable salts or solvates thereof.

In another embodiment, the present invention relates to compound of formula IV:

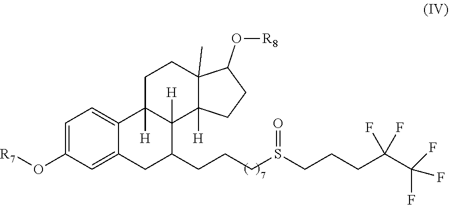

wherein R₇ is

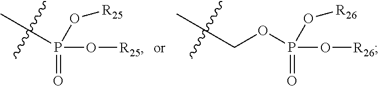

wherein each R₂₅ and R₂₆ is selected independently from hydrogen; optionally substituted alkyl, aryl or heteroaryl; and
R₈ is

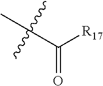

wherein R₁₇ is selected from NH₂, NHR₁₈ or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, —C(O)-aminoacid, carboxyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl; and enantiomers, diastereomers, racemates, pharmaceutically acceptable salts or solvates thereof.

In another embodiment, the present invention relates to compound of formula IV:

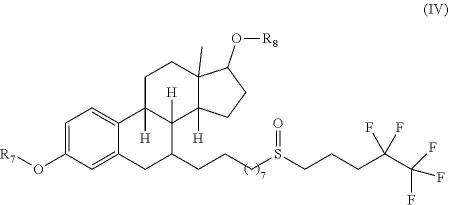

wherein R₇ is

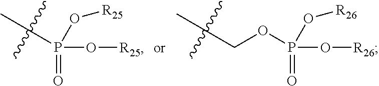

wherein each R₂₅ and R₂₆ is selected independently from hydrogen; optionally substituted alkyl, aryl or heteroaryl; and
R₈ is

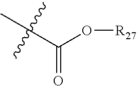

wherein R₂₇ is selected from group comprising optionally substituted alkyl, alkenyl, alkynyl, cycloalkyl, heterocycloalkyl, aryl, or heteroaryl; and enantiomers, diastereomers, racemates, pharmaceutically acceptable salts or solvates thereof.

In another embodiment, the present invention relates to compound of formula IV:

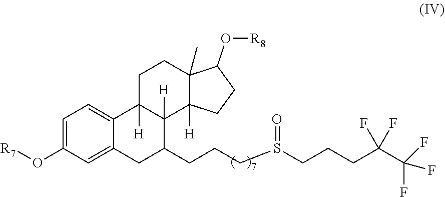

wherein R₇ is

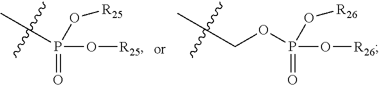

wherein each R₂₅ and R₂₆ is selected independently from hydrogen; optionally substituted alkyl, aryl or heteroaryl; and
R₈ is

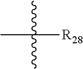

wherein R₂₈ is selected from group comprising optionally substituted alkyl, alkenyl, alkynyl, alkoxy, cycloalkyl, heterocycloalkyl, aryl or heteroaryl; wherein one or more substitution is selected from formyl, halo, phosphate, cyano, nitro, or amino; and enantiomers, diastereomers, racemates, pharmaceutically acceptable salts or solvates thereof.

In another embodiment, the present invention relates to compound of formula IV:

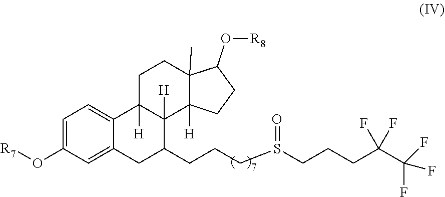

wherein R₇ is

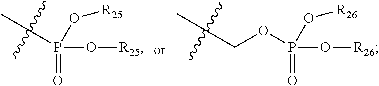

wherein each R₂₅ and R₂₆ is selected independently from hydrogen; optionally substituted alkyl, aryl or heteroaryl; and
R₈ is amino acid, wherein the amino acid is linked via ester linkage at the point of attachment; and
enantiomers, diastereomers, racemates, pharmaceutically acceptable salts or solvates thereof.

In another embodiment, the present invention relates to compound of formula V:

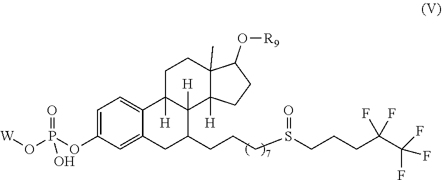

wherein W is selected from group comprising of hydrogen, or optionally substituted alkyl; and
R₉ is selected independently from group comprising of:
i)

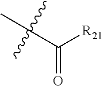

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, aryl, heteroaryl;
ii)

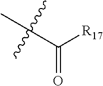

wherein R₁₇ is selected from NHR₁₈, or NR₁₉R₂₀; wherein R₁₈ is selected from group comprising optionally substituted alkyl, cycloalkyl, heterocycloalkyl, aryl and heteroaryl; R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the R₂₁ radicals are independently selected from the group consisting of optionally substituted alkyl, carboxyl, alkylcarbonyl, amino, —C(O)-aminoacid, cycloalkyl, aryl, aralkyl, heterocyclyoalkyl;
iii)

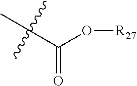

wherein R₂₇ is group comprising selected from optionally substituted alkyl, cycloalkyl, heterocycloalkyl, aryl, or heteroaryl; wherein the substitution can be amino;
iv)

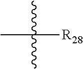

wherein R₂₈ is selected from group comprising optionally substituted alkyl, cycloalkyl, heterocycloalkyl, aryl or heteroaryl; wherein the substitution is amino;
v) amino acid, wherein the amino acid is linked via ester linkage at the point of attachment; and
enantiomers, diastereomers, racemates, pharmaceutically acceptable salts or solvates thereof.

In another embodiment, the present invention relates to compound of formula V-A:

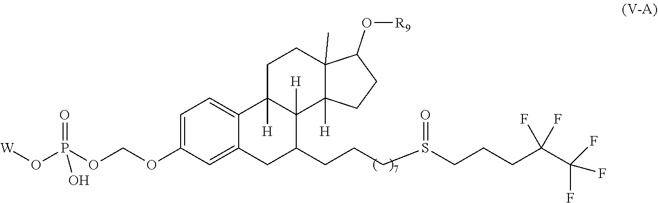

wherein W is selected from group comprising of hydrogen, or optionally substituted alkyl; and
R₉ is selected independently from group comprising of:
i)

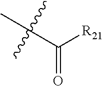

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, aryl, heteroaryl;
ii)

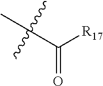

wherein R₁₇ is selected from NH₁₈, or NR₁₉R₂₀; wherein R₁₈ is selected from group comprising optionally substituted alkyl, cycloalkyl, heterocycloalkyl, aryl and heteroaryl; R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the R₂₁ radicals are independently selected independently from the group consisting of optionally substituted alkyl, carboxyl, alkylcarbonyl, amino, —C(O)-aminoacid, cycloalkyl, aryl, aralkyl, heterocyclyoalkyl;
iii)

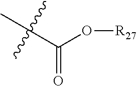

wherein R₂₇ is selected from group comprising optionally substituted alkyl, cycloalkyl, heterocycloalkyl, aryl, or heteroaryl; wherein the substitution can be amino;
iv)

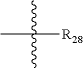

wherein R₂₈ is selected from group comprising optionally substituted alkyl, cycloalkyl, heterocycloalkyl, aryl or heteroaryl; wherein the substitution is amino;
v) amino acid, wherein the amino acid is linked via ester linkage at the point of attachment; and
enantiomers, diastereomers, racemates, pharmaceutically acceptable salts or solvates thereof.

In another embodiment, the present invention relates to compound selected from group consisting of:

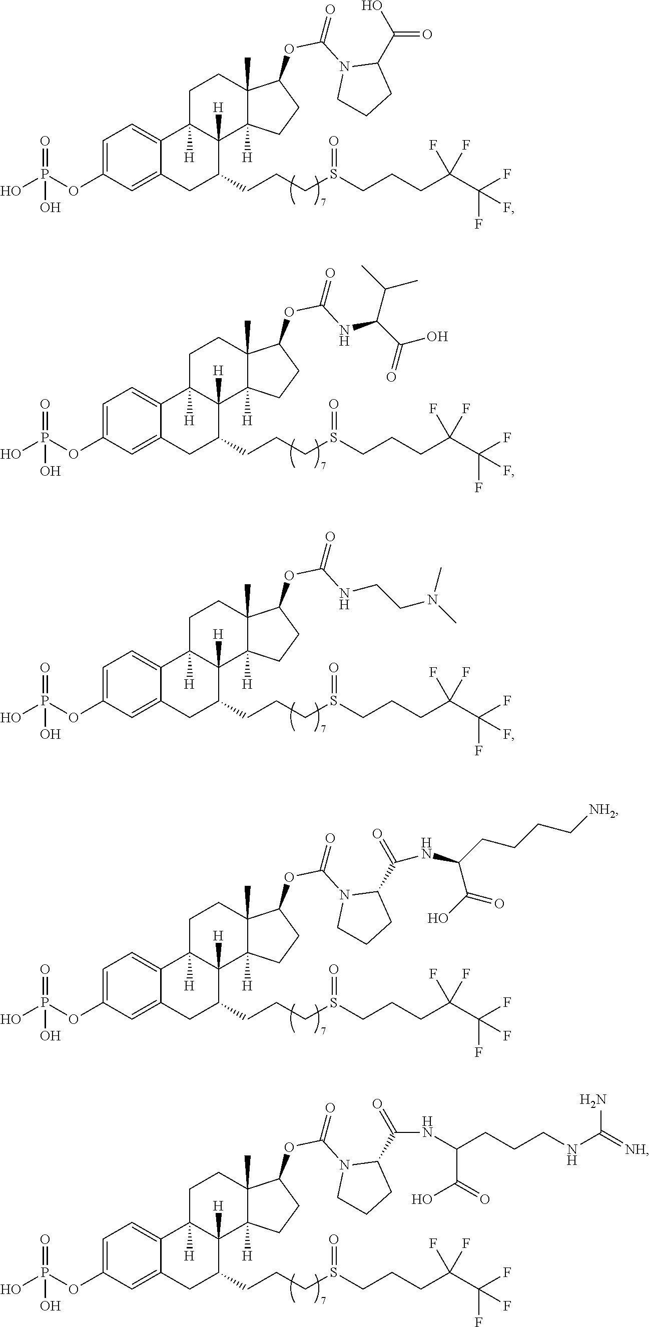

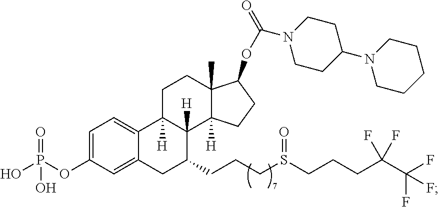

and pharmaceutically acceptable salts or solvates thereof.

In another embodiment, the present invention relates to compound:

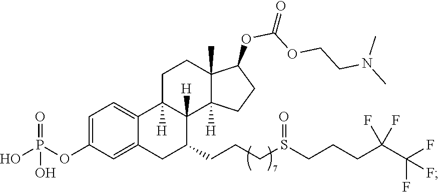

and pharmaceutically acceptable salts or solvates thereof.

In another embodiment, the present invention relates to compound selected from group consisting of:

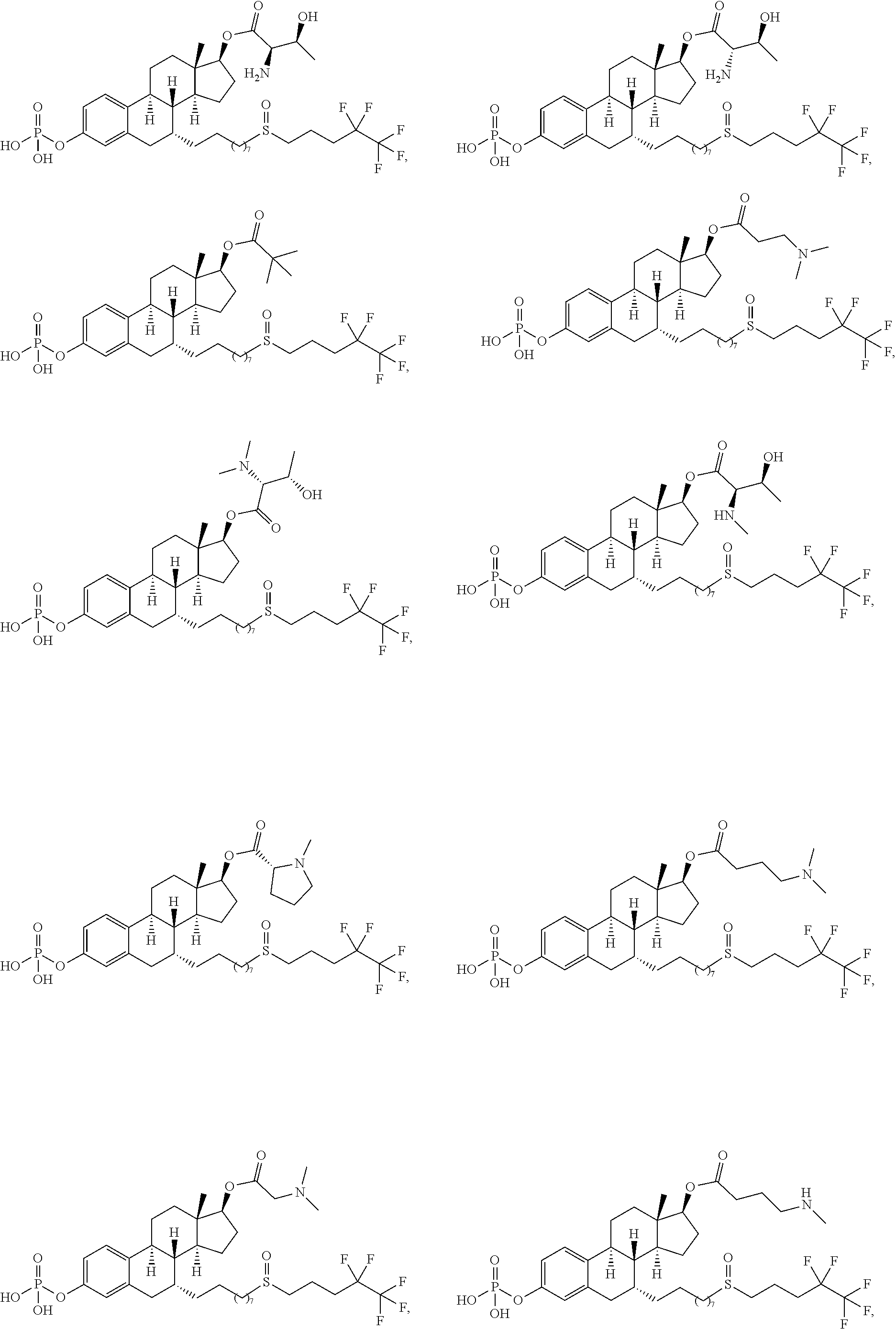

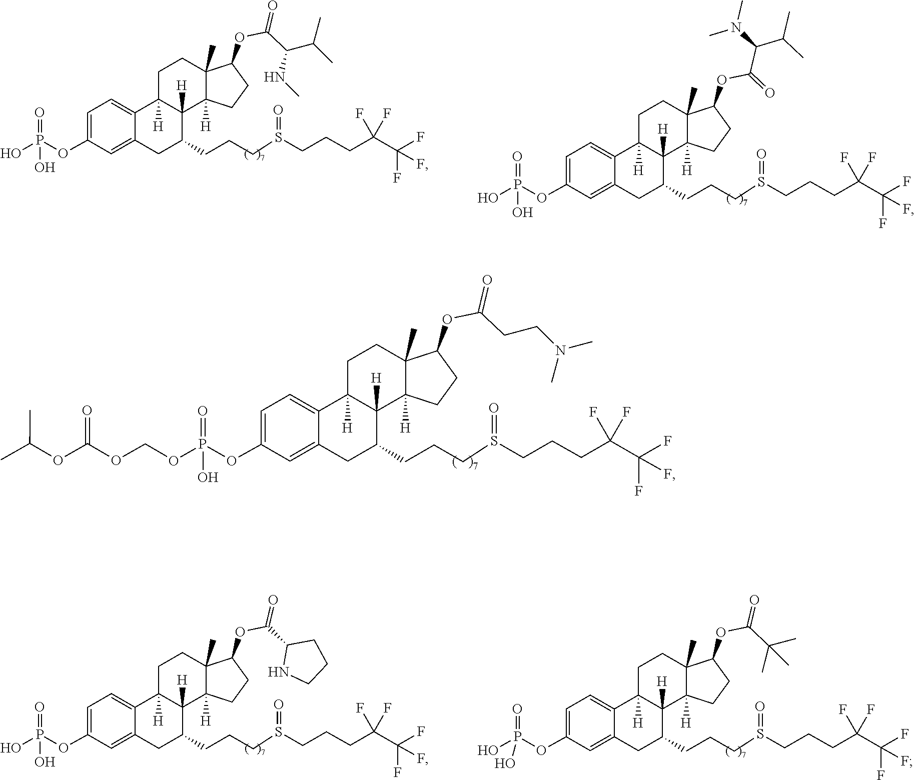

and pharmaceutically acceptable salts or solvates thereof.

In another embodiment, the present invention relates to compound:

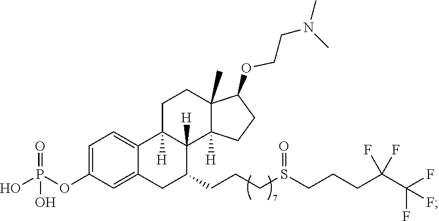

and pharmaceutically acceptable salts or solvates thereof.

In another embodiment, the present invention relates to compound of formula VI:

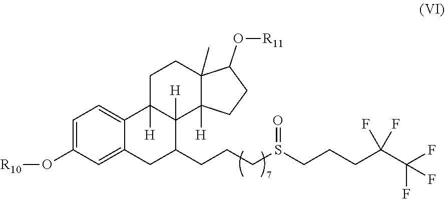

wherein, R₁₀ is

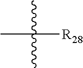

wherein R₂₈ is selected from group comprising optionally substituted alkyl, alkenyl, alkynyl, alkoxy, cycloalkyl, heterocycloalkyl, aryl, or heteroaryl; wherein one or more substitution is selected from formyl, halo, phosphate, cyano, nitro, amino, hydroxy, heterocycloalkyl, or alkoxy; and
R₁₁ is hydrogen or

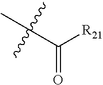

where R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
with the proviso that when R₁₁ is hydrogen, R₁₀ is not methyl or ethyl; and
enantiomers, diastereomers, racemates, pharmaceutically acceptable salts or solvates thereof.

In another embodiment, the present invention relates to compound of formula VII:

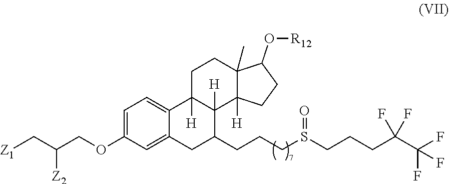

wherein, each Z₁ and Z₂ is independently selected from optionally substituted hydroxy, phosphate, or heterocycloalkyl; and
R₁₂ is hydrogen or

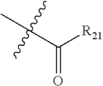

wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, alkoxycarbonyl, alkenyl, alkynyl, alkylthio, arylcarbonyl; and
enantiomers, diastereomers, racemates, pharmaceutically acceptable salts or solvates thereof.

In another embodiment, the present invention relates to compound of formula VII:

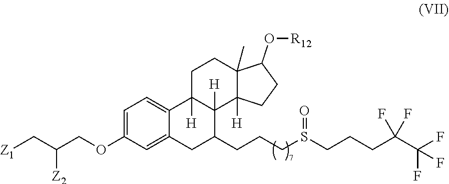

wherein, each Z₁ and Z₂ is independently selected from optionally substituted hydroxy, phosphate, or heterocycloalkyl; and
R₁₂ is hydrogen or

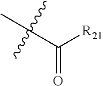

wherein R₂₁ is selected from group comprising optionally substituted alkyl, alkenyl, alkynyl, cycloalkyl, aryl, or heteroaryl; wherein one or more substitution is selected from alkyl, amino, or cycloalkyl; and
enantiomers, diastereomers, racemates, pharmaceutically acceptable salts or solvates thereof.

In another embodiment, the present invention relates to compound selected from group consisting of:

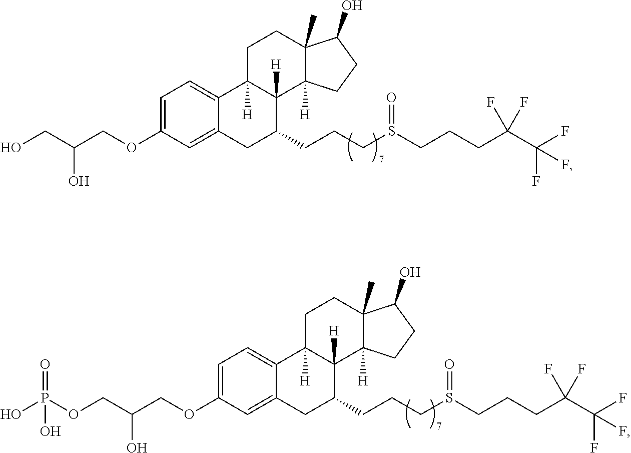

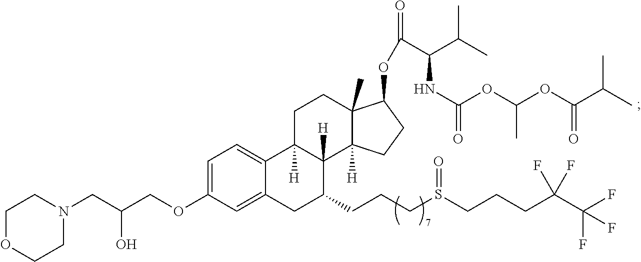

and pharmaceutically acceptable salts or solvates thereof.

In another embodiment, the present invention relates to compound of formula VIII:

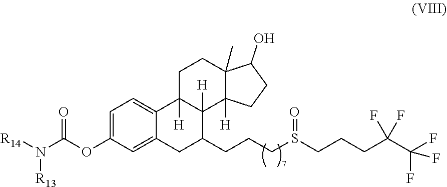

wherein R₁₃ are selected from group comprising H; optionally substituted alkyl, aryl, heteroaryl, heterocycloalkyl, cycloalkyl; and R₁₄ are selected from group comprising optionally substituted alkyl, aryl, heteroaryl, heterocycloalkyl, cycloalkyl; or R₁₃ and R₁₄ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, phosphonyl, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and; enantiomers, diastereomers, racemates, pharmaceutically acceptable salts or solvates thereof.

In another embodiment, the present invention relates to compound of formula VIII:

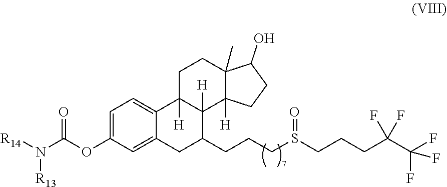

wherein R₁₃ and R₁₄ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of substituted alkyl, alkylcarbonyl, formyl, halo, haloalkyl, alkylphosphate, phosphate, phosphonyl, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and; enantiomers, diastereomers, racemates, pharmaceutically acceptable salts or solvates thereof. In one embodiment, one or more substitution on radical R₂₁ is selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate. In another embodiment, the present invention relates to compound of formula VIII:

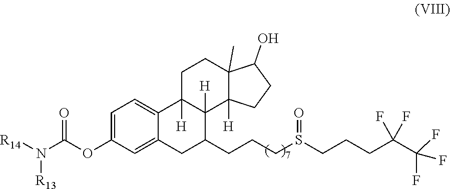

wherein R₁₃ and R₁₄ are taken together along with the nitrogen to which they are attached to form a three to seven membered saturated, partially unsaturated or unsaturated heterocyclic ring and substituted at a substitutable position with one or more R₂₁ radicals, wherein the one or more R₂₁ radicals are independently selected at each occurrence from the group consisting of substituted alkyl, alkylcarbonyl, formyl, halo, haloalkyl, alkylphosphate, phosphate, phosphonyl, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthio alkyl, arylcarbonyl, aralkylcarbonyl, alkoxyalkyl and; enantiomers, diastereomers, racemates, pharmaceutically acceptable salts or solvates thereof. In one embodiment, one or more substitution on radical R₂₁ is selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate.

In another embodiment, the present invention relates to compound of formula VIII:

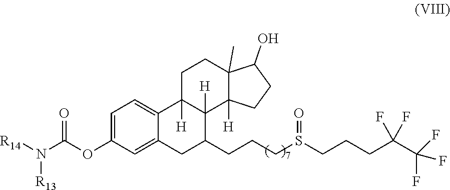

wherein R₁₃ is selected from hydrogen, alkyl; and
R₁₄ is selected from group comprising optionally substituted alkyl, heterocycloalkyl, or aryl; wherein one or more substitution is selected from alkyl, alkoxy, halo, heteroaryl, heterocycloalkyl, cycloalkyl, amino, or hydroxy; and
enantiomers, diastereomers, racemates, pharmaceutically acceptable salts or solvates thereof.

In another embodiment, the present invention relates to compound selected from group consisting of:

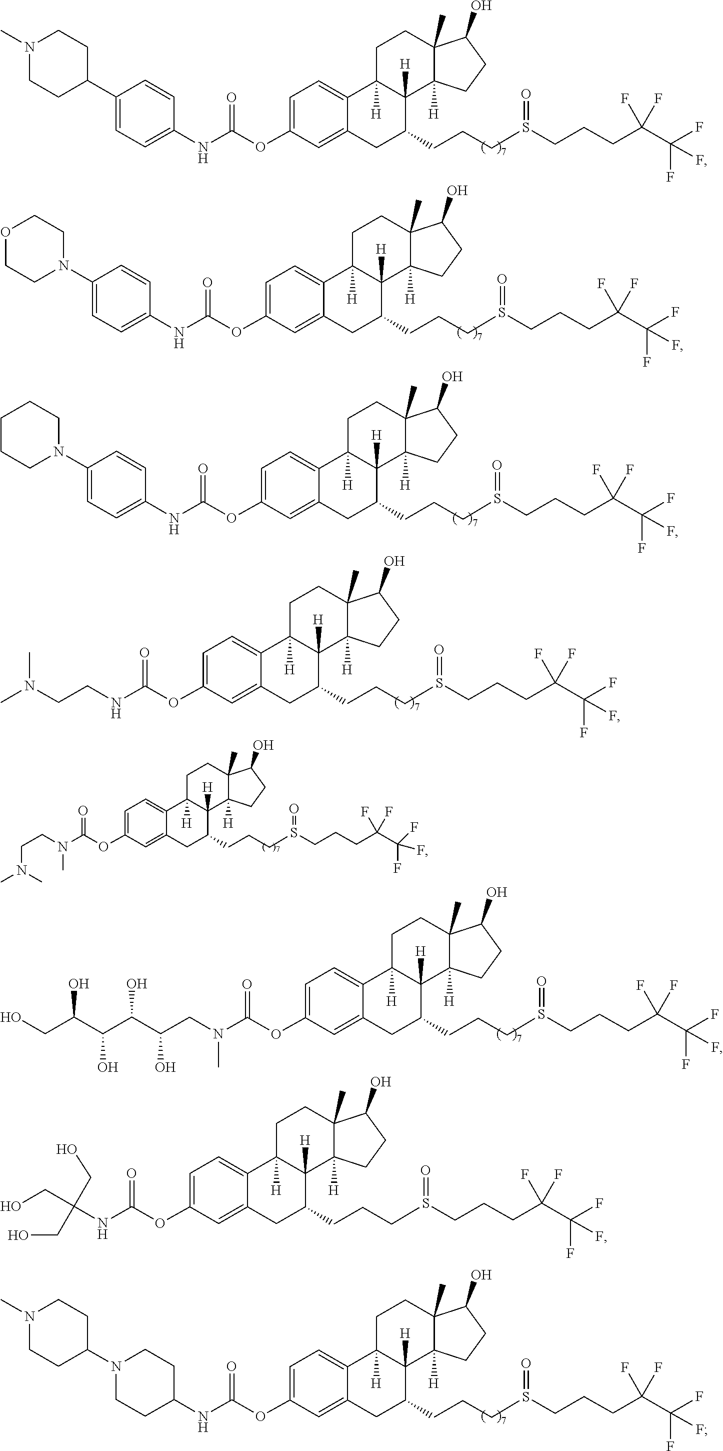

and pharmaceutically acceptable salts or solvates thereof.
In another embodiment, the present invention relates to prodrugs of Fulvestrant of formula IX:

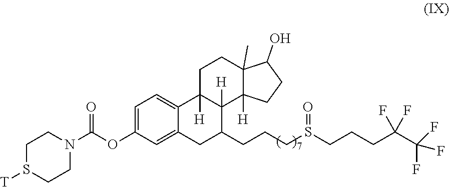

wherein S is selected from O, C, or N; and
T is selected from group comprising optionally substituted aryl, heteroaryl, heterocycloalkyl, cycloalkyl, amino,

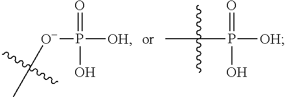

wherein one or more substitution is selected from alkyl, alkoxy; and enantiomers, diastereomers, racemates, pharmaceutically acceptable salts or solvates thereof.

In another embodiment, the present invention relates to compound selected from group consisting of:

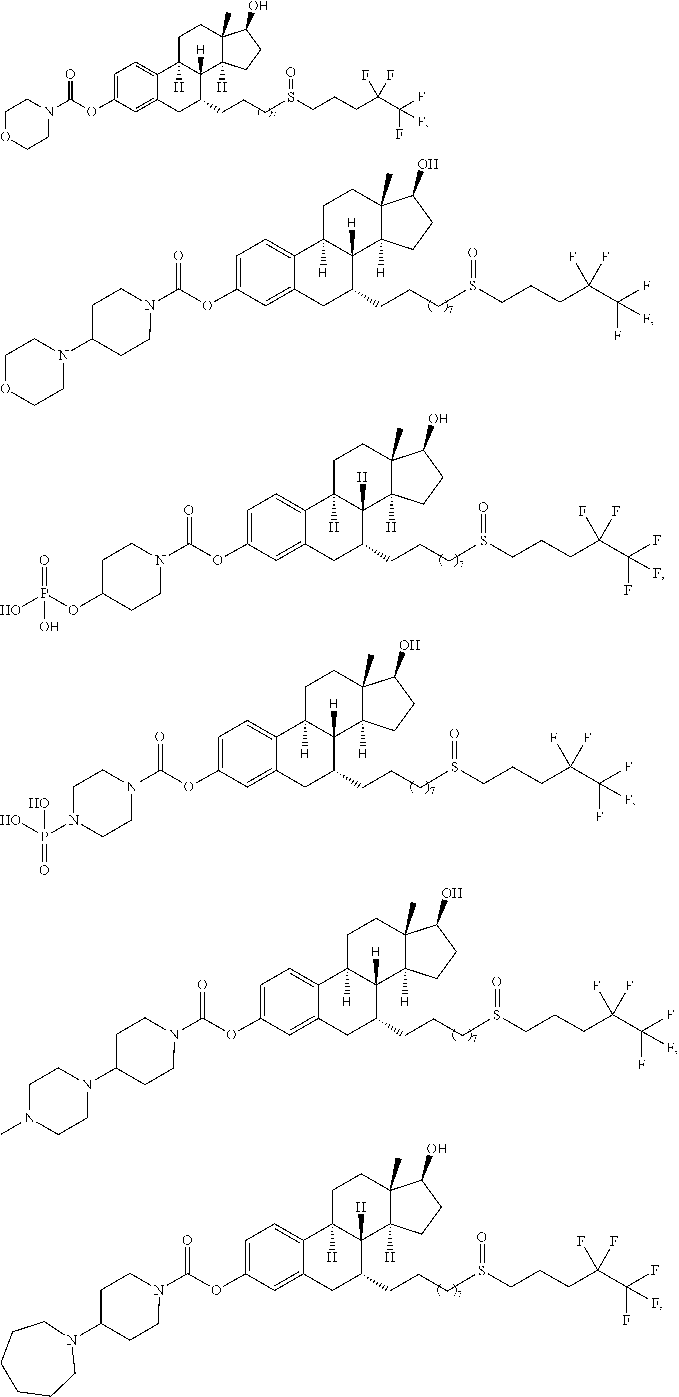

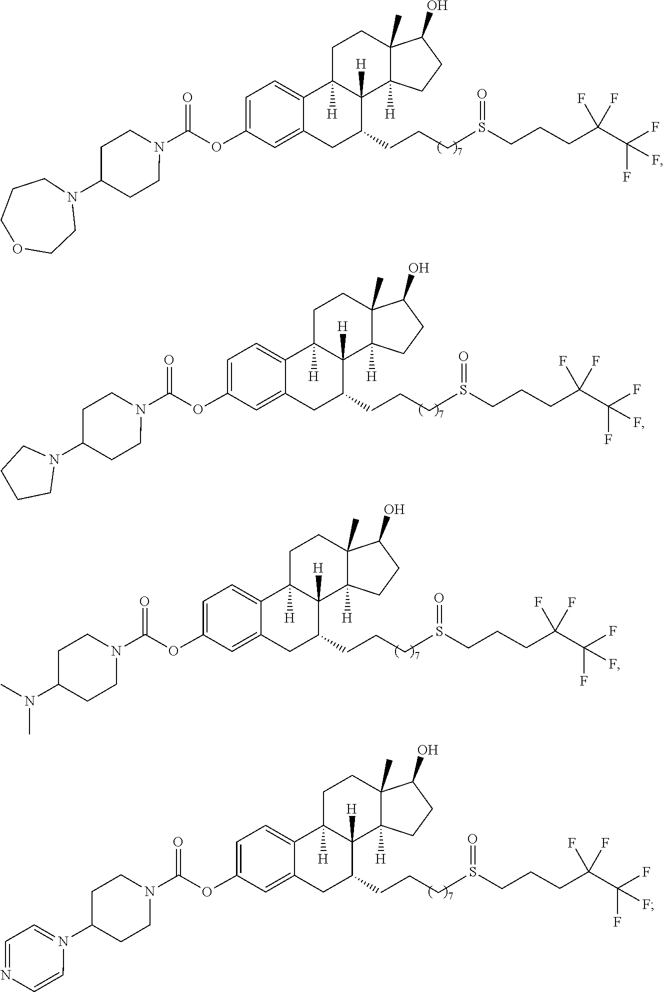

and pharmaceutically acceptable salts or solvates thereof.

In another embodiment, the present invention relates to prodrugs of Fulvestrant of formula X:

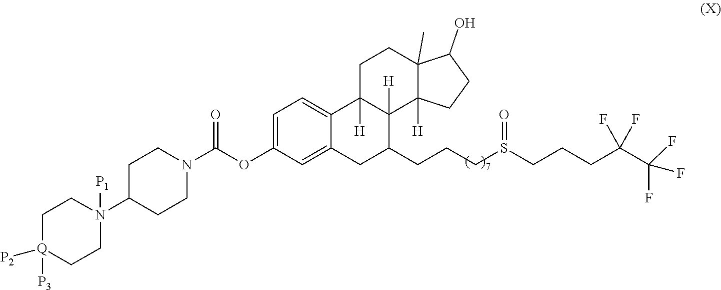

wherein, Q is selected from C or N; and
when Q is C, P₁ is selected independently from optionally substituted alkyl, optionally substituted

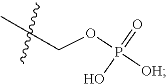 [SMILES: *COP(=O)(O)O]

wherein substitution on

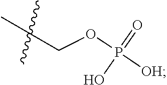 [SMILES: *COP(=O)(O)O]

is alkyl; and P₂ and P₃ is selected independently from hydrogen or alkyl; and
when Q is N, P₂ is selected independently from optionally substituted alkyl, optionally substituted

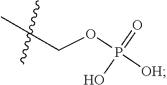 [SMILES: *COP(=O)(O)O]

wherein substitution on

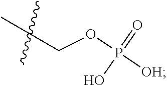 [SMILES: *COP(=O)(O)O]

is alkyl; P₁ is hydrogen and P₃ is optionally substituted alkyl; and
enantiomers, diastereomers, racemates, pharmaceutically acceptable salts or solvates thereof.

In another embodiment, the present invention relates to compound selected from the group consisting of:

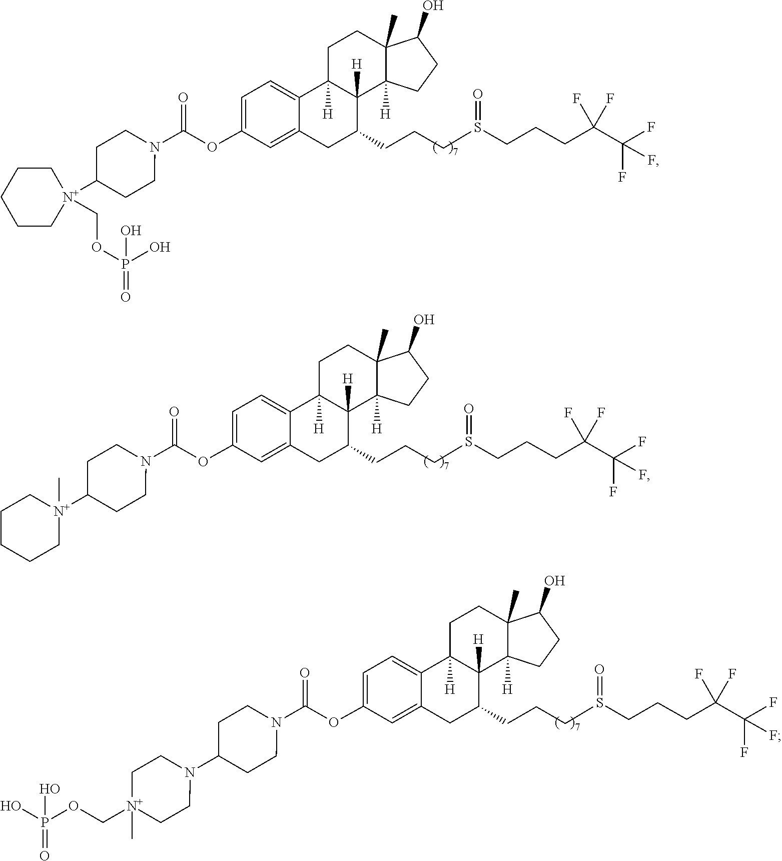

and pharmaceutically acceptable salts or solvates thereof.

In another embodiment, the present invention relates to compound of formula XI:

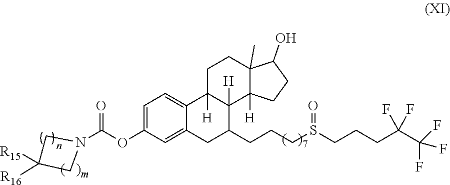

wherein R₁₅ and R₁₆ together forms a cyclic structure; wherein the said cyclic structure is selected from optionally substituted cycloalkyl, or heterocycloalkyl; wherein one or more substitution is selected from alkyl, alkoxy, halo, amino, or hydroxy; m and n can be independently 1 to 3; and enantiomers, diastereomers, racemates, pharmaceutically acceptable salts or solvates thereof.

In a preferred embodiment, the present invention relates to compound of formula XI where m and n both are 1 or 2.

In another embodiment, the present invention relates to compound selected from the group consisting of:

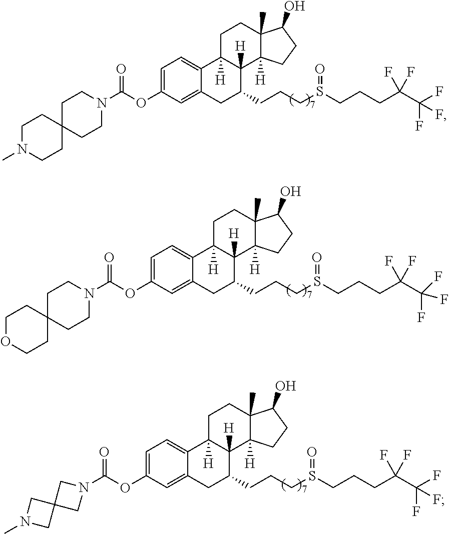

and pharmaceutically acceptable salts, solvates thereof.

In another embodiment, the present invention relates to compound selected from group consisting of:

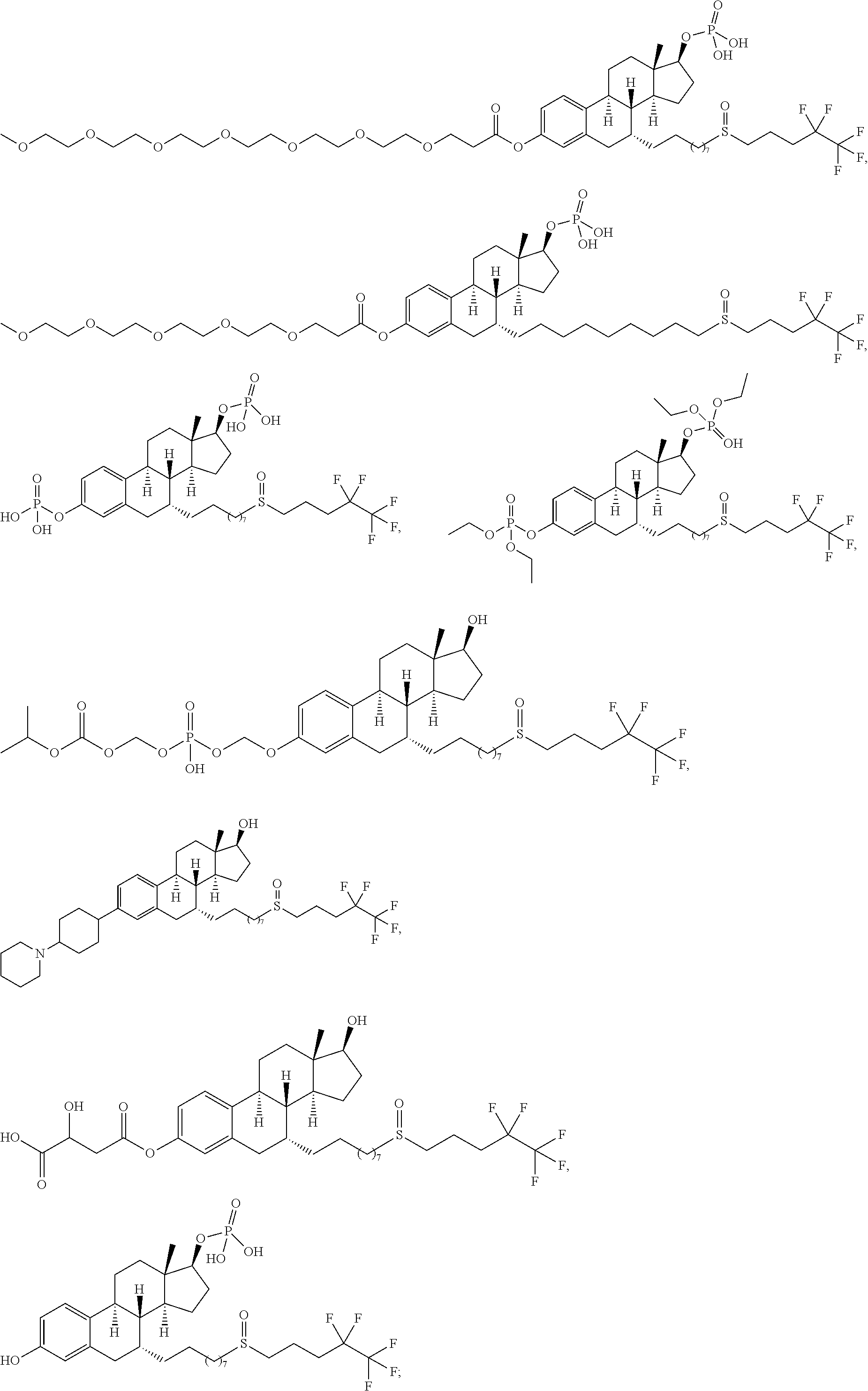

and pharmaceutically acceptable salts or solvates thereof.

In another embodiment, the present invention relates to meglumine salt of compound

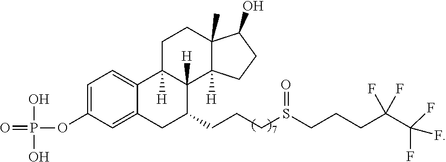

In another embodiment, the present invention relates to dimeglumine salt of compound

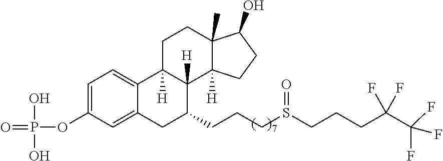

In another embodiment, the present invention relates to compound

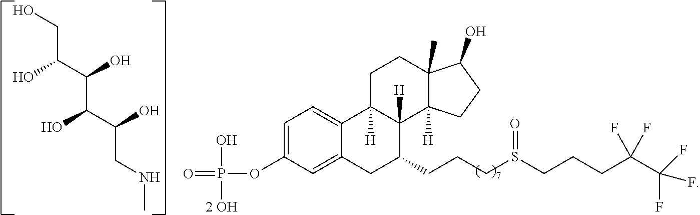

In another embodiment, the compounds of the present invention further can be delivered in a form of pharmaceutical composition comprising compound of invention and enantiomers, diastereomers, racemates, pharmaceutically acceptable salts or solvates thereof, and pharmaceutically acceptable excipients.

In another embodiment, the present invention relates to pharmaceutical composition comprising Fulvestrant prodrug and enantiomers, diastereomers, racemates, pharmaceutically acceptable salts or solvates thereof, and pharmaceutically acceptable excipients.

The Fulvestrant prodrug for the said pharmaceutical composition is selected from the compound of formula I, formula II, formula III, formula IV, formula V, formula VI, formula VII, formula VIII, formula IX, formula X and formula XI; and enantiomers, diastereomers, racemates, pharmaceutically acceptable salts and solvates thereof.

The pharmaceutical compositions of the present disclosure can be in any form known to those of skill in the art. The pharmaceutical compositions of this invention may be administered orally, parenterally, by inhalation spray, topically, rectally, nasally, buccally or via an implanted reservoir, preferably oral administration or administration by injection.

For instance, in some embodiments the pharmaceutical composition comprising desired product is formulated for oral delivery. In embodiment the pharmaceutical composition comprising desired product is selected from a group consisting of a concentrate, dried powder, liquid, capsule, pellet, and pill.

For instance, in some embodiments the pharmaceutical composition comprising desired product is for parenteral. In embodiment the pharmaceutical composition comprising desired product is selected from a group consisting of a intravenous injection, intramuscular injection, subcutaneous injection, powder for solution for injection, powder for suspension for injection, liposome, oily injection, sustained release particles.

The pharmaceutical compositions disclosed herein may also further comprise carriers, binders, diluents, and excipients.

In an embodiment, the disclosure provides for a pharmaceutical composition in the form of Fulvestrant prodrug for treatment of diseases and/or symptoms that are meant to be treated by the original drug molecule. The composition may comprise Fulvestrant prodrug in an amount that is as therapeutically effective as or more therapeutically effective than the original drug.

In another embodiment, the invention provides use of compounds of the present invention in the preparation of a pharmaceutical formulation as describe hereinabove, for the treatment of a benign or malignant disease of the breast or reproductive tract, preferably treating breast cancer.

In another embodiment, the invention provides compounds for the treatment of a benign or malignant disease of the breast or reproductive tract, preferably treating breast cancer.

In another embodiment, the invention provides Fulvestrant prodrugs useful in the treatment of oestrogen-dependent indications such as breast cancer and gynaecological conditions, such as endometriosis.

The Fulvestrant prodrugs for the said treatment of a benign or malignant disease of the breast or reproductive tract is selected from the compound of formula I, formula II, formula III, formula IV, formula V, formula V-A, formula VI, formula VII, formula VIII, formula IX, formula X and formula XI.

In another embodiment, the invention provides a method of treating a benign or malignant diseases of the breast or reproductive tract, preferably treating breast cancer, comprising administration of a compound selected from formula I, formula II, formula III, formula IV, formula V, formula VI, formula VII, formula VIII, formula IX, formula X, formula XI; and enantiomers, diastereomers, racemates, pharmaceutically acceptable salts and solvates thereof.

In another embodiment, the invention provides a method of treating a benign or malignant diseases of the breast or reproductive tract, preferably treating breast cancer, comprising administration of a compound selected from formula I, formula II, formula III, formula IV, formula V, formula V-A, formula VI, formula VII, formula VIII, formula IX, formula X, formula XI; and enantiomers, diastereomers, racemates, pharmaceutically acceptable salts and solvates thereof.

Also, in other embodiment, the present disclosure relates to new Fulvestrant prodrug and any stereochemically isomeric form, hydrate, solvate or pharmaceutically acceptable salt thereof; either alone or in combination with at least one additional therapeutic agent, in the treatment of diseases and/or symptoms meant to be treated by the original drugs. The combination with an additional therapeutic agent may take the form of combining the new Fulvestrant prodrug compounds with any known therapeutic agent.

The following examples are given for the purpose of illustrating the present invention and should not be considered as limiting the scope of the invention.

### EXAMPLE-01

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-(piperidin-1-yl)phenyl)carbamate

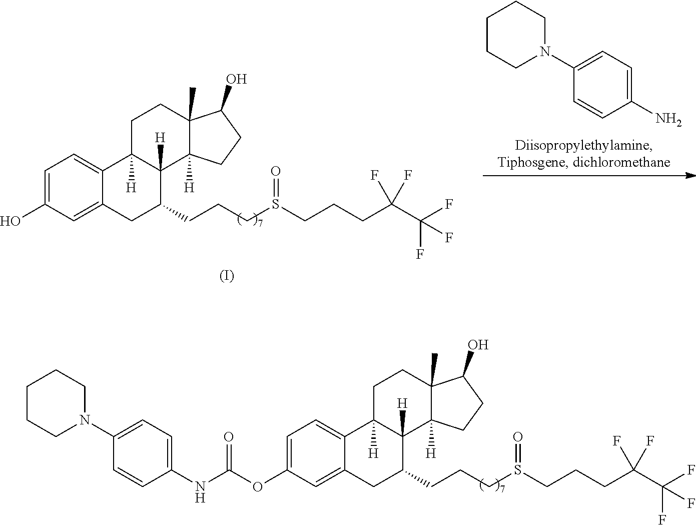

To a stirred solution of Fulvestrant (0.5 g) in dichloromethane (7 mL) were added diisopropylethylamine (0.21 mL) and triphosgene (0.122 g) at 0° C. and stirred the reaction mixture for 10 min at the same temperature. After 10 min, 4-(piperidin-1-yl)aniline (0.218 g) was added in the reaction mixture at 0° C. and the reaction mixture was stirred at room temperature for 2 h. After completion of reaction on TLC, the reaction mass was diluted with water (5 mL) and extracted with dichloromethane (3×40 mL). The combined organic layer was dried over anhydrous sodium sulphate and evaporated under reduced pressure to obtained crude material. The crude was purified by prep HPLC and the product fractions were lyophilized to give 0.060 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-(piperidin-1-yl)phenyl)carbamate as a white colour solid.

LCMS: 99.69% (m/z: 809.59, [M+1]⁺, 214 nm).

¹H NMR (400 MHz , DMSO-d₆): δ 9.85 (s, 1H), 7.30 (m, 3H), 6.89 (m, 4H), 4.49 (s, 1H), 3.54 (m, 1H), 3.03 (t, J=5.2 Hz, 4H), 2.87-2.80 (m, 2H), 2.75-2.70 (m, 3H), 2.68-2.66 (m, 1H), 2.50-2.49 (m, 2H), 2.38-2.29 (m, 2H), 1.92-1.88 (m, 3H), 1.82 (d, J=12.4 Hz, 1H), 1.75 (m, 1H), 1.63-1.58 (m, 7H), 1.52-1.49 (m, 3H), 1.38-1.20 (m, 18H) 0.9 (m, 1 H), 0.68 (s, 3H).

### EXAMPLE-02

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(phosphonooxy)piperidine-1-carboxylate

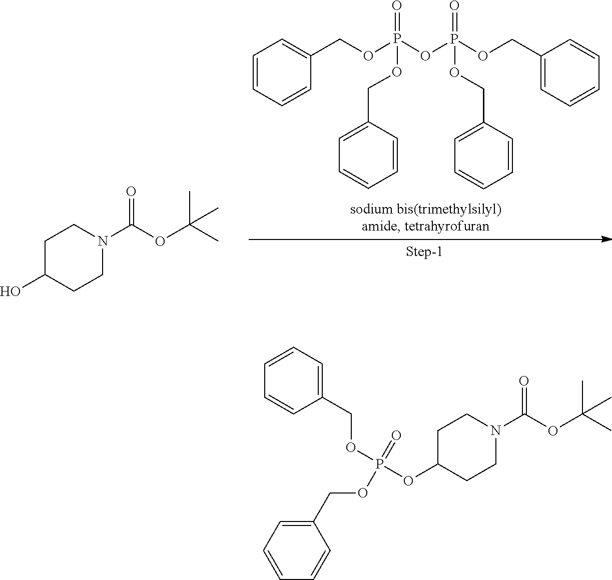 [SMILES: O=P(OCc1ccccc1)(OCc1ccccc1)OP(=O)(OCc1ccccc1)OCc1ccccc1 ; CC(C)(C)OC(=O)N1CCC(O)CC1 ; CC(C)(C)OC(=O)N1CCC(OP(=O)(OCc2ccccc2)OCc2ccccc2)CC1]

### Step-1: Preparation of tert-butyl 4-((bis(benzyloxy)phosphoryl)oxy)piperidine-1-carboxylate

To a stirred solution of tert-butyl 4-hydroxypiperidine-1-carboxylate (1 g) in tetrahydrofuran (20 mL) was cooled to −10° C. and then added 1M sodium bis(trimethylsilyl)amide (NaHMDS) in tetrahydrofuran (9.9 mL) and stirred the reaction mixture for 30 min at the same temperature. Tetrabenzyl diphosphate (5.34 g) was then added and the reaction mixture was again stirred at room temperature for 16 h. After completion of the reaction on TLC, the mixture was diluted with water and extracted with ethyl acetate, dried the organic layer over sodium sulfate and concentrated under reduced pressure to obtain the crude material (2.1 g, crude LCMS purity 70%) as a light yellow solid. The crude proceeded next step without further purification.

LCMS purity: 70.00%

### Step-2: Preparation of Dibenzyl piperidin-4-yl phosphate (Trifluoroacetate salt)

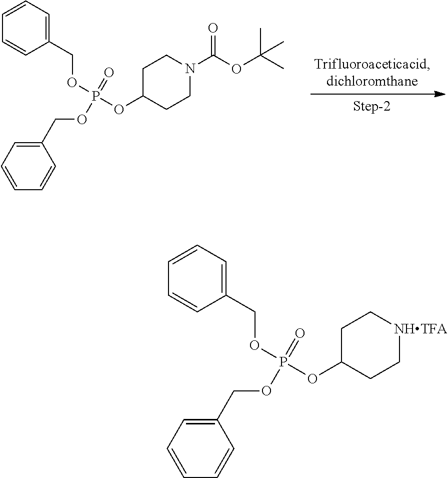

To a stirred solution of tert-butyl 4-((bis(benzyloxy)phosphoryl)oxy)piperidine-1-carboxylate (above crude 1 g) in dichloromethane (40 mL) at 0° C. was added trifluoroacetic acid (6 mL) and stirred for 2 h at the same temperature. After completion of the reaction on TLC, the mixture was concentrated under reduced pressure to obtain the crude material (1.0 g, crude LCMS purity 63.6%) as a sticky yellow solid. The crude proceeded next step without further purification.

LCMS purity: 63.60%

### Step-3: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-((bis(benzyloxy)phosphoryl)oxy)piperidine-1-carboxylate

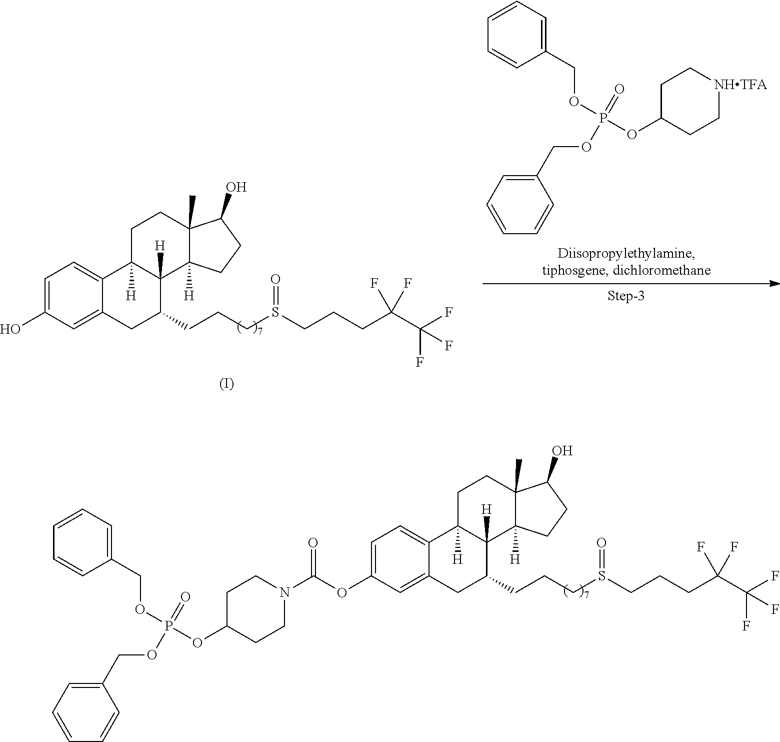

To a stirred solution of (7R,8R,9S,13S,14S,17S)-13-methyl-7-(9-((4,4,5,5,5-pentafluoropentyl)sulfinyl)nonyl)-7,8,9,11,12,13,14,15,16,17-decahydro-6H-cyclopenta[a]phenanthrene-3,17-diol (1 g) in dichloromethane (20 mL) was added diisopropylethylamine (0.55 mL) and triphosgene (0.245 mL) at 0° C. and the mixture was stirred at same temperature for 10 min. Then dibenzyl piperidin-4-yl phosphate (trifluoroacetate salt) (0.9 g) was added and the mixture was stirred for 16 h at room temperature. After completion of the reaction by TLC, the reaction mixture was diluted with water and extracted with dichloromethane (2×50 mL), dried the organic layer over sodium sulfate and concentrated under reduced pressure to obtained the crude material (1.1 g, crude LCMS purity 27%) as a colourless thick oil. The crude proceeded next step without further purification.

LCMS purity: 27.00%

### Step-4: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(phosphonooxy)piperidine-1-carboxylate

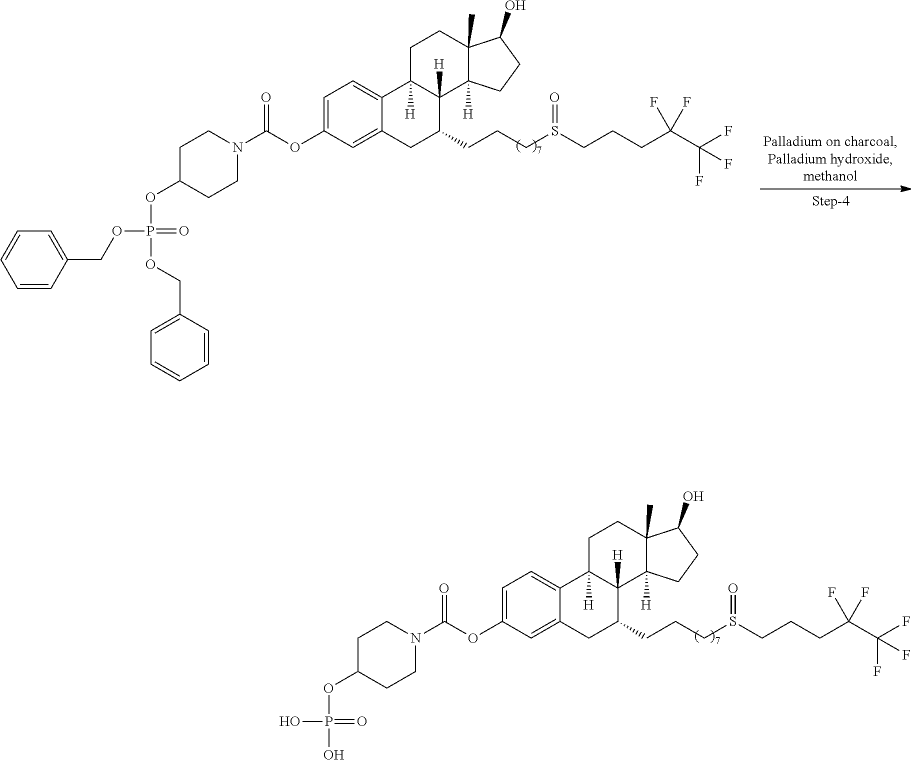

To a stirred solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-((bis(benzyloxy)phosphoryl)oxy)piperidine-1-carboxylate (above crude 0.68 g) in methanol (40 mL) was added 10% palladium on charcoal:10% palladium hydroxide (1:1, 540 mg) and the reaction mixture was stirred at room temperature for 1 h. After completion of the reaction by TLC, the reaction mixture was filtered through celite and filtrate was concentrated under reduced pressure to obtain the crude material (0.6 g, crude LCMS purity 44%). The crude was purified by prep HPLC and the product fractions were lyophilized to give 0.047 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (phosphonooxy)piperidine-1-carboxylate as a white solid.

LCMS: 97.8% (m/z: 812.22, [M+1]⁺, 214 nm).

¹H NMR (400 MHz, DMSO-d₆): 6 7.22 (d, J=8.8 Hz, 1H), 6.79 (d, J=8.4 Hz, 1H), 6.73 (d, J=2 Hz, 1H), 4.24 (bd, J=3.6 Hz,1H), 3.74 (bs, 1H), 3.67 (bs, 1H), 3.51 (t, J=8.4 Hz, 1H), 3.36 (bs, 1H), 3.22 (bs, 1H), 2.83-2.75 (m, 2H), 2.74-2.61 (m, 4H), 2.24-2.21 (m, 4H), 1.92-1.77 (m, 6 H), 1.71-1.62 (m, 1H), 1.62-1.42 (m, 6H), 1.41-1.01 (m, 19H), 0.81 (bs, 1H), 0.64 (s, 3H).

### EXAMPLE-03

### Preparation of (7R,8R,9S,13S,14S,17S)-13-methyl-7-(9-((4,4,5,5,5-pentafluoropentyl)sulfinyl)nonyl)-3-(phosphonooxy)-7,8,9,11,12,13,14,15,16,17-decahydro-6H-cyclopenta[a]phenanthren-17-yl 3-(dimethylamino)propanoate

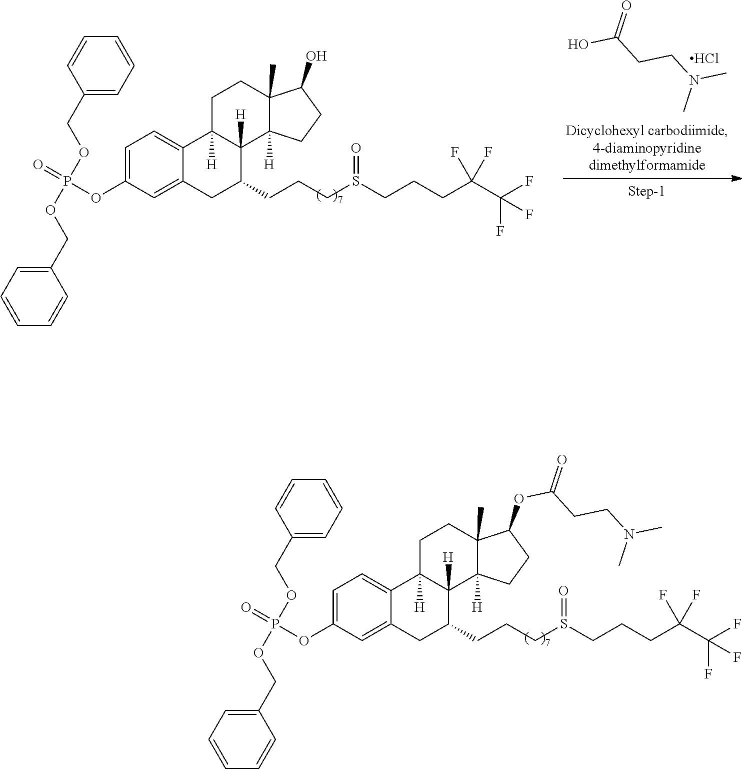

### Step-1: Preparation of (7R,8R,9S,13S,14S,17S)-3-((bis(benzyloxy)phosphoryl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-17-yl 3-(dimethylamino)propanoate

To a stirred solution of dibenzyl ((7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl) phosphate (1 g) in dimethylformamide (10 mL) were added dicyclohexyl carbodiimide (0.712 g) and 4-dimethylaminopyridine (0.07 g) at 0° C. After 20 min, 3-(dimethylamino)propanoic acid hydrogen chloride (0.529 g) was added and the resulting mixture was stirred at room temperature for 4 h. After completion of reaction by LCMS, the reaction mixture was diluted with water and extracted with ethyl acetate. The combined organic layer was dried over sodium sulphate and concentrated under reduced pressure to obtain the crude material (0.75 g, Crude LCMS purity 38%) as yellow sticky solid which was used next step without further purification.

LCMS purity: 38.00%

### Step-2: Preparation of (7R,8R,9S,13S,14S,17S)-13-methyl-7-(9-((4,4,5,5,5-pentafluoropentyl)sulfinyl)nonyl)-3-(phosphonooxy)-7,8,9,11,12,13,14,15,16,17-decahydro-6H-cyclopenta[a]phenanthren-17-yl 3-(dimethylamino)propanoate

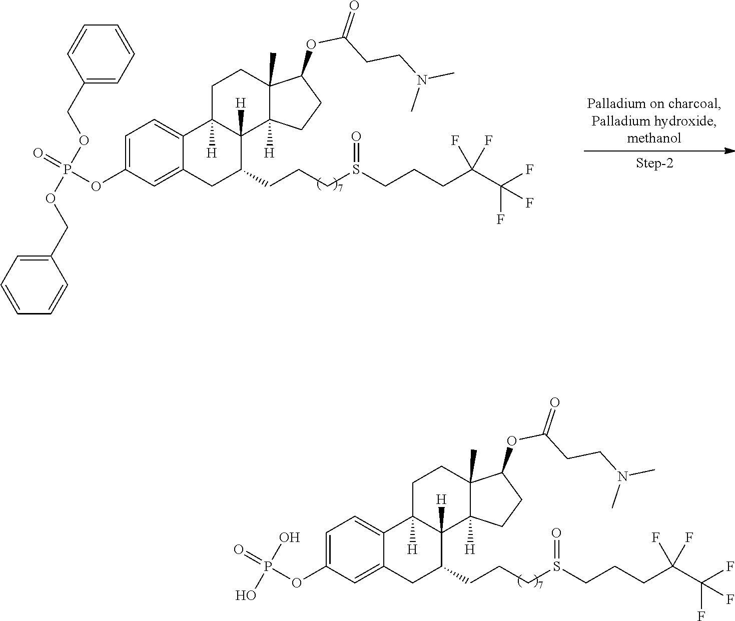

To a stirred solution of (7R,8R,9S,13S,14S,17S)-3-((bis(benzyloxy)phosphoryl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-17-yl 3-(dimethylamino)propanoate (above crude 0.75 g) in methanol (50 mL) was added 10% palladium on charcoal:10% palladium hydroxide (1:1, 0.6 g) and the reaction mixture was stirred at room temperature for 1 h. After completion of the reaction by TLC, the reaction mixture was filtered through celite bed and filtrate was concentrated under reduced pressure to obtain the crude material (0.510 g, LCMS purity 69%). The crude was purified by prep HPLC and the product fractions were lyophilized to give 0.13 g of (7R,8R,9S,13S,14S,17S)-13-methyl-7-(9-((4,4,5,5,5-pentafluoropentyl)sulfinyl) nonyl)-3-(phosphonooxy)-7,8,9,11,12,13,14,15,16,17-decahydro-6H-cyclopenta[a]phenanthren-17-yl-3-(dimethylamino)propanoate as a white solid.

LCMS: 90.2% (m/z: 786.32 [M+1]⁺, 214 nm).

¹H NMR (400 MHz, DMSO-d₆): δ 7.07 (d, J=8 Hz, 1H), 6.83 (s, 1H), 6.82 (d, J=7.8 Hz, 1H), 4.65 (t, J=8.4 Hz, 1H), 2.86-2.81 (m, 3H), 2.76-2.61 (m, 4H), 2.63-2.61 (m, 2H), 2.43-2.33 (m, 10H), 2.28-2.20 (m, 2H), 2.10 (bs, 1H), 1.92-1.88 (m, 2H), 1.65-1.56 (m, 5H), 1.51-1.42 (m, 3H), 1.38-1.17 (m, 17H), 0.91 (bs, 1H), 0.64 (s, 3H).

### EXAMPLE-04

### Preparation of (7R,8R,9S,13S,14S,17S)-13-methyl-7-(9-((4,4,5,5,5-pentafluoropentyl)sulfinyl)nonyl)-3-(phosphonooxy)-7,8,9,11,12,13,14,15,16,17-decahydro-6H-cyclopenta[a]phenanthren-17-yl [1,4′-bipiperidine]-1′-carboxylate

### Step-1: Preparation of (7R,8R,9S,13S,14S,17S)-3-((bis(benzyloxy)phosphoryl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-17-yl [1,4′-bipiperidine]-1′-carboxylate

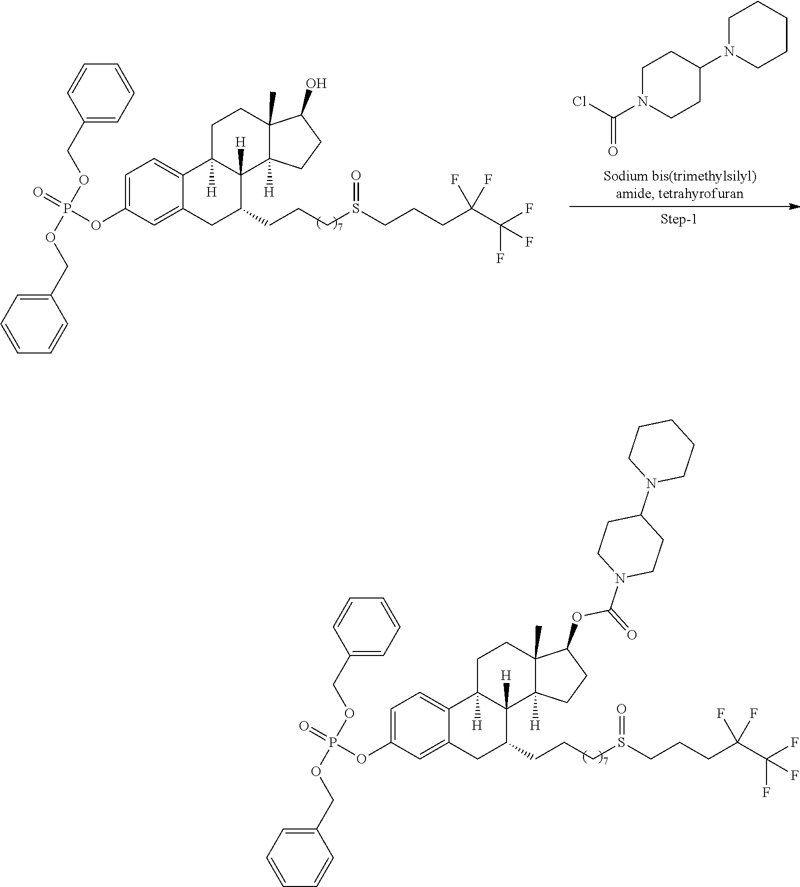

To a solution of dibenzyl ((7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl) phosphate (0.300 g) in tetrahydrofuran (5 mL) was added sodium bis(trimethylsilyl)amide (0.7 mL) at 0° C. The resulting mixture was stirred at the same temperature for 30 min. Then added [1,4′-bipiperidine]-1′-carbonyl chloride (0.160 g) at room temperature and the reaction mixture was stirred at room temperature for 16 h. After completion of reaction by LCMS, the reaction mixture was diluted with water and extracted with ethyl acetate. The combined organic layer was dried over sodium sulphate and concentrated under reduced pressure to obtain the crude material (320 mg 35% pure by LCMS) as a brown sticky solid which was used next step without further purification.

LCMS purity: 35.00%

### Step-2: Preparation of (7R,8R,9S,13S,14S,17S)-13-methyl-7-(9-((4,4,5,5,5-pentafluoropentyl)sulfinyl)nonyl)-3-(phosphonooxy)-7,8,9,11,12,13,14,15,16,17-decahydro-6H-cyclopenta[a]phenanthren-17-yl [1,4′-bipiperidine]-1′-carboxylate

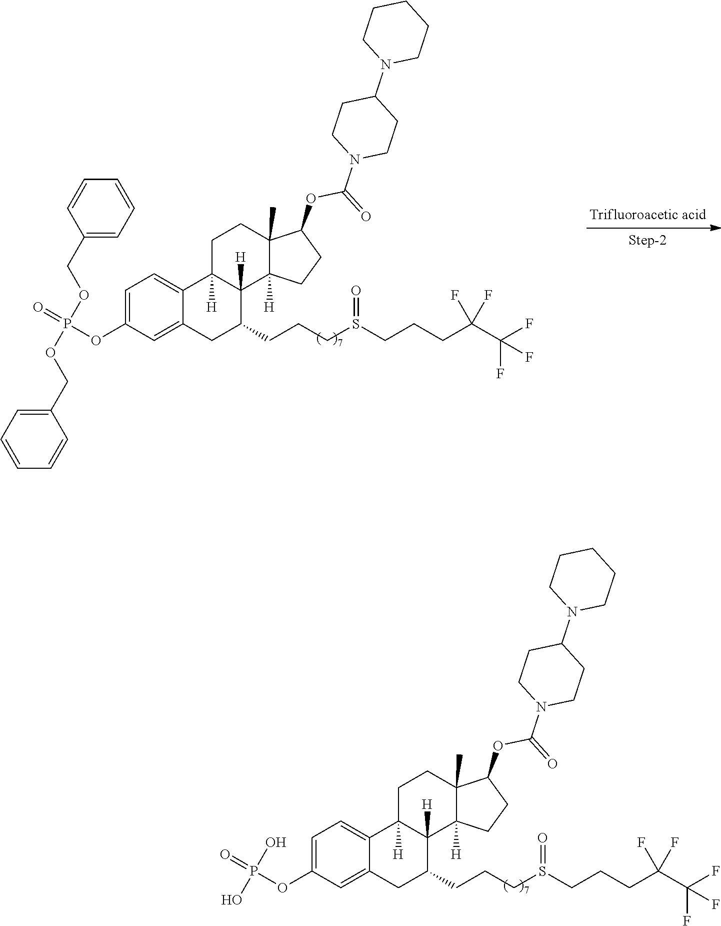

A solution of (7R,8R,9S,13S,14S,17S)-3-((bis(benzyloxy)phosphoryl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-17-yl [1,4′-bipiperidine]-1′-carboxylate (step-1 crude 0.250 mg) in trifluoroacetic acid (6 mL) was stirred at 70° C. for 2 h. After completion of reaction by LCMS, reaction mass was cooled to room temperature and concentrated under reduced pressure to obtain the crude material (200 mg, 19% pure by LCMS). The crude was purified by prep HPLC and the product fractions were lyophilized to give 0.02 g of (7R,8R,9S,13S,14S,17S)-13-methyl-7-(9-((4,4,5,5,5-pentafluoropentyl)sulfinyl)nonyl)-3-(phosphonooxy)-7,8,9,11,12,13,14,15,16,17-decahydro-6H-cyclopenta[a]phenanthren-17-yl [1,4′-bipiperidine]-1′-carboxylate as a white colour solid.

LCMS: 96.7% (m/z: 881.52, [M+1]⁺, 214 nm).

¹H NMR (400 MHz, DMSO-d₆): δ 7.16 (bs, 1H), 6.75 (bs, 2H), 4.08-4.06 (m, 3H), 2.99-2.50 (m, 10H), 2.39-2.32 (m, 5H), 2.03-1.86 (m, 7H), 1.79-1.50 (m, 14H), 1.33-1.21 (m, 20H), 0.92 (m, 1H), 0.66 (s, 3H).

### EXAMPLE-05

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-methyl((2S,3R,4R,5R)-2,3,4,5,6-pentahydroxyhexyl)carbamate

### Step-1: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate

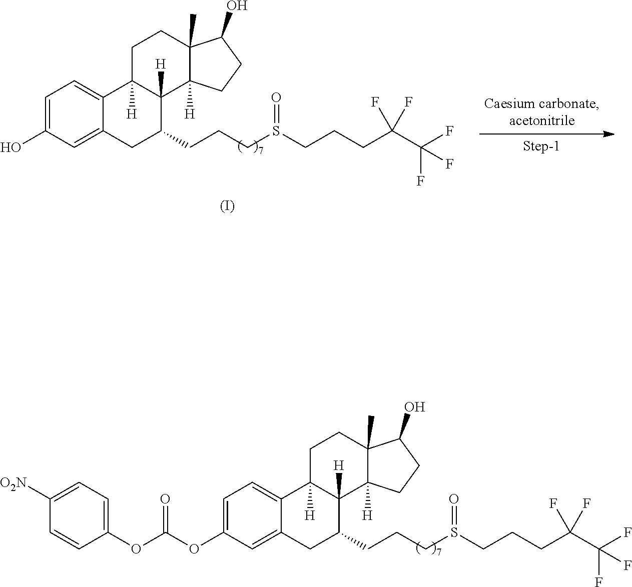

To a stirred solution of Fulvestrant (2.0 g) in acetonitrile (20 mL) were added caesium carbonate (3.24 g) and 4-nitrophenyl chloroformate (0.99 g) at room temperature and stirred the reaction mixture for 15 min at the same temperature. After completion of the reaction on TLC, the reaction mass was diluted with water (15 mL) and extracted with ethyl acetate (2×30 mL), combined organic layer was dried over anhydrous sodium sulphate, and concentrated the organics under reduced pressure to give 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (2.3 g crude, LCMS 60%), which was used for next step without further purification.

LCMS purity: 60.00%

### Step-2: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-methyl((2S,3R,4R,5R)-2,3,4,5,6-pentahydroxyhexyl)carbamate

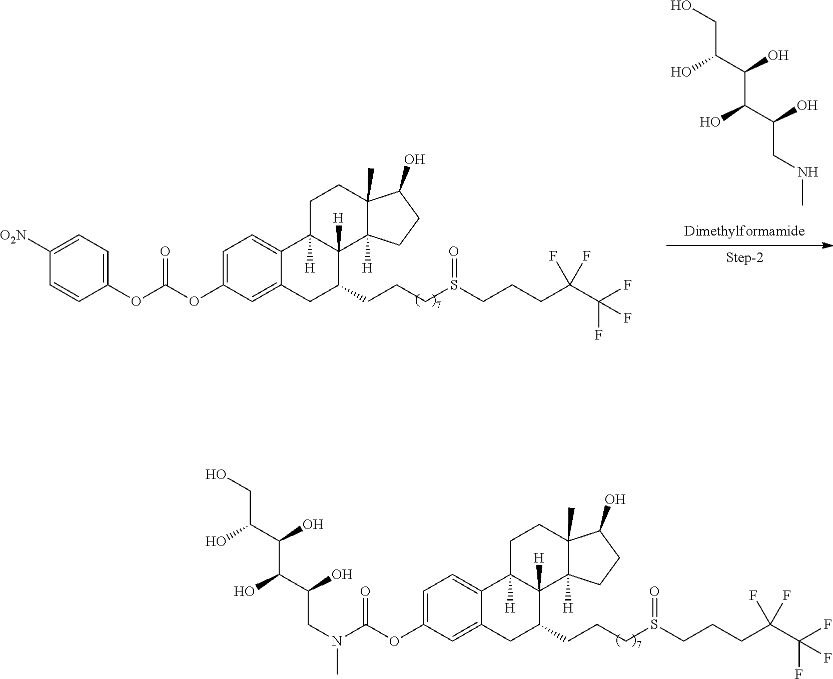

To a stirred solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl)carbonate (0.5 g) in dimethylformamide (3 mL) was added (2R,3R,4R,5S)-6-(methyl amino)hexane-1,2,3,4,5-pentaol (0.370 g) at room temperature and stirred the reaction mixture for 2 h at the same temperature. After completion of the reaction on TLC, the reaction mass was diluted with ice water (15 mL) and extracted with ethyl acetate (2×30 mL). The organic layer was combined, dried over sodium sulfate, and concentrated the organic layer under reduced pressure to give 0.510 g crude material, which was purified by PREP-HPLC and the product fractions were lyophilized to give 0.072 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-ylmethyl((2S,3R,4R,5R)-2,3,4,5,6-pentahydroxyhexyl)carbamate as a white colour solid.

LCMS: 99.41% (m/z: 828.34, [M+1]⁺, 214 nm).

¹H NMR (400 MHz, DMSO-d₆): δ 7.25 (d, J=8.4 Hz, 1H), 6.82-6.81 (d, J=8.4 Hz, 1H), 6.76 (bs, 1H), 4.89-4.81 (m, 1H), 4.52 (s, 1H), 4.50 (d, J=4.8 Hz, 1H), 4.30-445 (m, 3H), 3.89-3.82 (m, 1H), 3.58-3.52 (m, 3H), 3.46-3.37 (m, 4H), 3.32-3.29 (m, 1H), 3.05-2.92 (m, 3H), 2.87-2.60 (m, 6 H), 2.42-2.37 (m , 2H), 2.33-2.24 (m, 2H), 1.94-1.85 (m, 3H), 1.80 (d, J=12 Hz, 1H), 1.71-1.68 (bs, 1H), 1.63-1.47 (m, 4H), 1.38-1.18 (m, 18H), 0.90 (bs, 1H), 0.67 (s, 3H).

### EXAMPLE-06

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-(1,3-dihydroxy-2-(hydroxymethyl)propan-2-yl)carbamate

### Step-1: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate

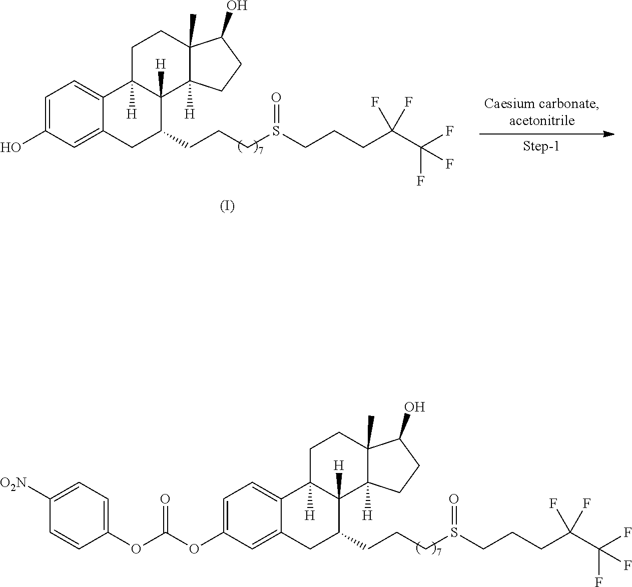

To a stirred solution of Fulvestrant (2.0 g) in acetonitrile (20 mL) were added caesium carbonate (3.24 g) and 4-nitrophenyl chloroformate (0.99 g) at room temperature and stirred the reaction mixture for 15 min at the same temperature. After completion of the reaction on TLC, the reaction mass was diluted with water (15 mL) and extracted with ethyl acetate (2×30 mL), combined organic layer was dried over anhydrous sodium sulphate, and concentrated the organics under reduced pressure to give 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (2.3 g crude, LCMS 60%), which was used for next step without further purification.

LCMS purity: 60.00%

### Step-2: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-(1,3-dihydroxy-2-(hydroxymethyl)propan-2-yl)carbamate

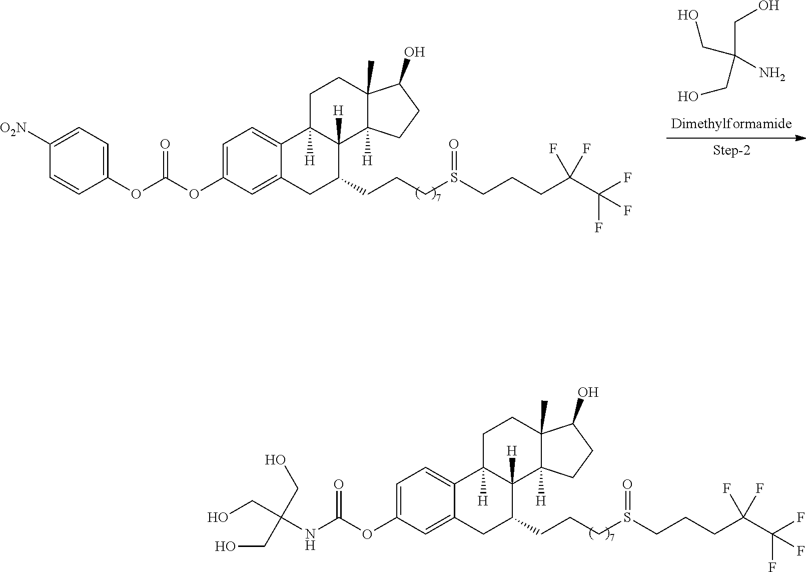

To a stirred solution of 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl)carbonate (0.5 g) in dimethylformamide (3 mL) was added 2-amino-2-(hydroxymethyl)propane-1,3-diol (0.156 g) at room temperature and stirred the reaction mixture for 2 h at the same temperature. After completion of reaction on TLC, the reaction mass was diluted with ice water (15 mL) and extracted with ethyl acetate (2×30 mL). The combined organic layer was dried over anhydrous sodium sulphate and concentrated the organics under reduced pressure to give 0.510 g crude material, which was purified by Prep-HPLC and the product fraction was lyophilized to give 0.140 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-(1,3-dihydroxy-2-(hydroxymethyl)propan-2-yl)carbamate as a white solid.

LCMS: 99.18% (m/z: 754.35, [M+1]⁺, 214 nm).

¹H NMR (400 MHz, DMSO-d₆): δ 7.25 (d, J=8.4 Hz, 1H), 6.82 (dd, J=2, 8.4 Hz, 1H), 6.77 (d, J=2 Hz, 1H), 6.68 (s, 1H), 4.53-4.49 (m, 4H), 3.57-3.54 (m, 7H), 2.87-2.80 (m, 2H), 2.76-2.71 (m, 2H), 2.67-2.62 (m, 1H), 2.43-2.37 (m, 2H), 2.32-2.23. (m, 2H), 1.94-1.86 (m, 3H), 1.80 (d, J=12 Hz, 1H), 1.71-1.68 (m, 1H), 1.63-1.56 (m, 4H), 1.38-1.18 (m, 19 H), 0.90-0.88 (m, 1H), 0.67 (s, 3H)

### EXAMPLE-07

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(azepan-1-yl)piperidine-1-carboxylate

### Step-1: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate

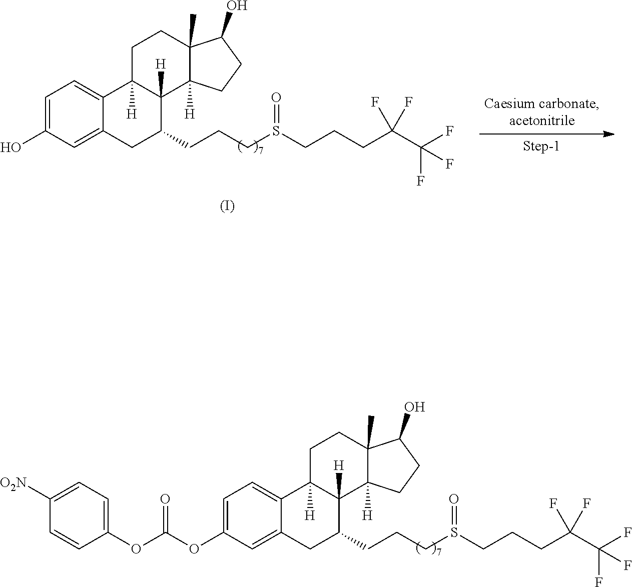

To a stirred solution of Fulvestrant (2.0 g) in acetonitrile (20 mL) were added caesium carbonate (3.24 g) and 4-nitrophenyl chloroformate (0.99 g) at room temperature and stirred the reaction mixture for 15 min at the same temperature. After completion of the reaction on TLC, the reaction mass was diluted with water (15 mL) and extracted with ethyl acetate (2×30 mL), combined organic layer was dried over anhydrous sodium sulphate, and concentrated the organics under reduced pressure to give 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (2.3 g crude, LCMS 60%), which was used for next step without further purification.

LCMS purity: 60.00%

### Step-2: Preparation of (7R,8R,9S,13S,14S,17S)-17-hydroxy-13-methyl-7-(9-((4,4,5,5,5-pentafluoropentyl)sulfinyl) nonyl)-7,8,9,11,12,13,14,15,16,17-decahydro-6H-cyclopenta[a]phenanthren-3-yl 4-(azepan-1-yl)piperidine-1-carboxylate

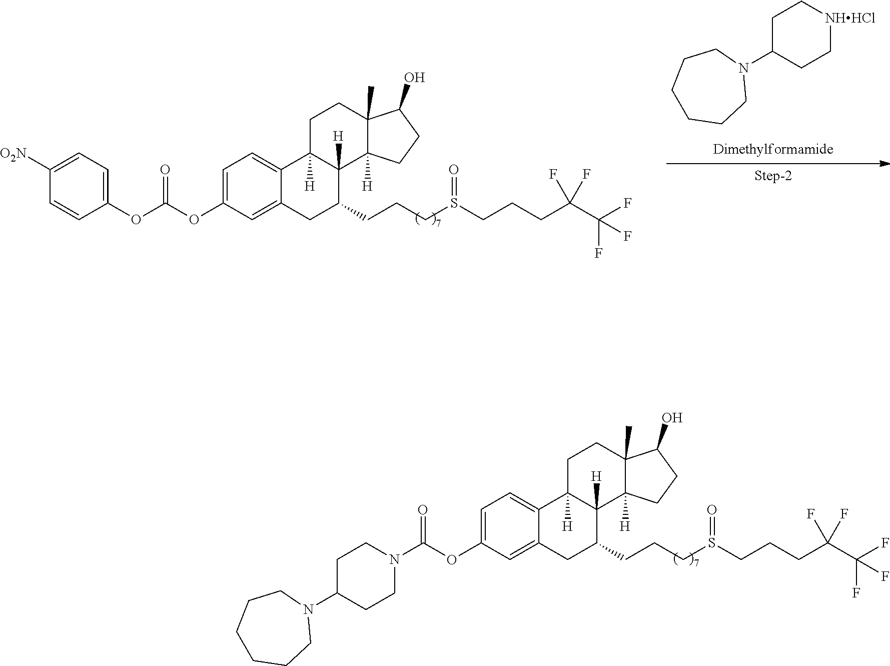

To a stirred solution of 1-(piperidin-4-yl)azepane hydrochloride (0.212 g) in acetonitrile (5 mL) was added potassium carbonate (0.88 g) and stirred at room temperature for 2 h. After that (7R,8R,9S,13S,14S,17S)-17-hydroxy-13-methyl-7-(9-((4,4,5,5,5-pentafluoropentyl)sulfinyl) nonyl)-7,8,9,11,12,13,14,15,16,17-decahydro-6H-cyclopenta[a]phenanthren-3-yl (4-nitrophenyl) carbonate (0.500 g) was added and reaction mass was stirred at room temperature for 30 min. After completion of the reaction on TLC, the reaction mass was diluted with ice water (15 mL) and extracted with ethyl acetate (2×30 mL). The combined organic layer was dried over anhydrous sodium sulphate and concentrated under reduced pressure to give 0.580 g crude material. The crude material was purified by Prep-HPLC and the product fractions were lyophilized to give 0.082 g of (7R,8R,9S,13S,14S,17S)-17-hydroxy-13-methyl-7-(9-((4,4,5,5,5-pentafluoropentyl)sulfinyl) nonyl)-7,8,9,11,12,13,14,15,16,17-decahydro-6H-cyclopenta[a]phenanthren-3-yl-4-(azepan-1-yl)piperidine-1-carboxylate as a white colour solid.

LCMS: 99.07% (m/z: 815.48, [M+1]⁺, 214 nm).

¹H NMR (400 MHz, DMSO-d₆): δ 7.25 (d, J=8.4 Hz, 1H), 6.82-6.80 (m, 1H), 6.77 (s, 1H), 4.50 (bs, 1H), 3.95-4.15 (m, 2H), 3.54 (t, J=8 Hz, 1H), 2.95-2.92 (m, 1H), 2.87-2.76 (m, 4H), 2.74-2.65 (m, 2H), 2.62-2.61 (m, 5H), 2.43-2.36 (m, 2H), 2.32-2.24 (m, 2H), 1.94-1.86 (m, 3H), 1.65-1.85 (m, 6H), 1.63-1.52 (m, 12H), 1.38-1.15 (m, 20H), 0.90-0.87 (m, 1H), 0.67 (s, 3H).

### EXAMPLE-08

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl(1′-methyl-[1,4′-bipiperidin]-4-yl)carbamate

### Step-1: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate

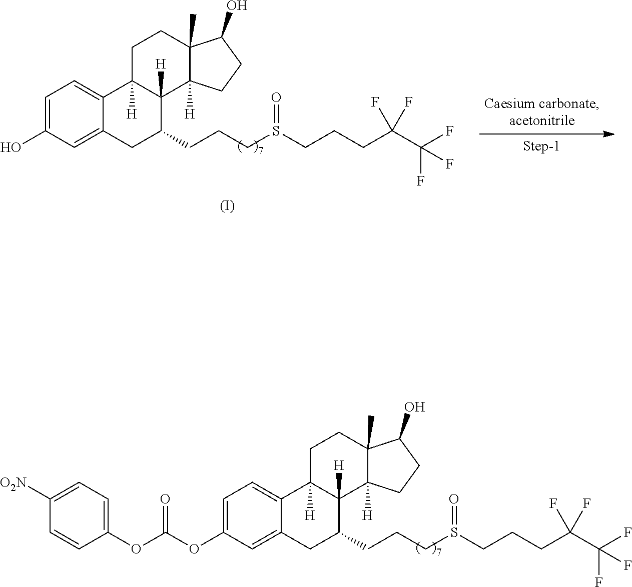

To a stirred solution of Fulvestrant (2.0 g) in acetonitrile (20 mL) were added caesium carbonate (3.24 g) and 4-nitrophenyl chloroformate (0.99 g) at room temperature and stirred the reaction mixture for 15 min at the same temperature. After completion of the reaction on TLC, the reaction mass was diluted with water (15 mL) and extracted with ethyl acetate (2×30 mL), combined organic layer was dried over anhydrous sodium sulphate, and concentrated the organics under reduced pressure to give 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (2.3 g crude, LCMS 60%), which was used for next step without further purification.

LCMS purity: 60.00%

### Step-2: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl(1′-methyl-[1,4′-bipiperidin]-4-yl)carbamate

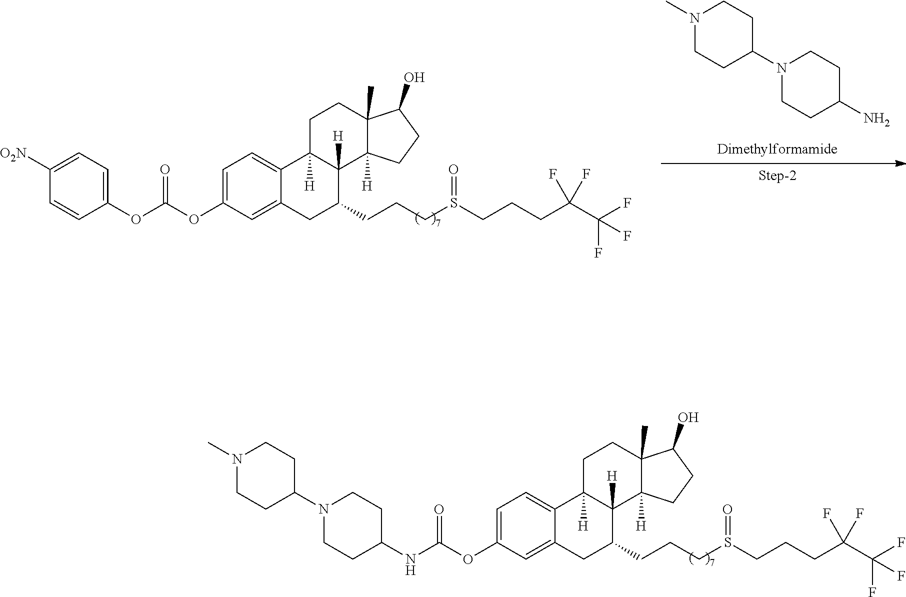

To a stirred solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (0.5 g) in ACN (7 mL) were added potassium carbonate (0.223 mg) followed by 1′-methyl-[1,4′-bipiperidin]-4-amine (0.191 mg) and stirred the reaction mixture for 1 h at room temperature. The reaction mixture was monitored by TLC (mobile phase: 70% ethyl acetate in Hexane). After completion of the reaction, the mixture was diluted with water and extracted with ethyl acetate (3×120 mL). The combined organic layer was dried over anhydrous sodium sulphate and evaporated under reduced pressure to obtained crude material. The crude was purified by prep HPLC and the product fractions were lyophilized to give 0.197 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl(1′-methyl-[1,4′-bipiperidin]-4-yl)carbamate as a white colour solid.

LCMS: 99.23% (m/z: 830.58, [M+1]⁺, 214 nm).

¹H NMR (400 MHz, DMSO-d₆): δ 7.65 (d, J=8 Hz, 1H), 7.24 (d, J=8.4 Hz, 1H), 6.79 (dd, J=2, 8 Hz, 1H), 6.75 (s, 1H), 4.49 (bs, 1H), 3.54 (t, J=8 Hz, 1H), 3.24 (m, 1H), 2.83-2.67 (m, 10H), 2.50-2.29 (m, 4H), 2.15-2.10 (m, 6H), 1.92-1.77 (m, 10 H), 1.75-1.56 (m, 7H), 1.56-1.23 (m, 22H), 1.17 (m, 1H), 0.67 (s, 3H)

### EXAMPLE-09

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(pyrrolidin-1-yl)piperidine-1-carboxylate

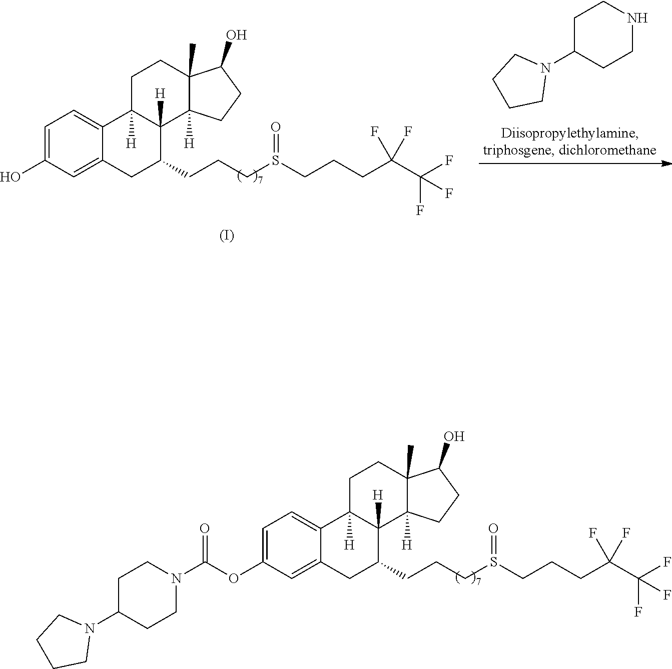

To a stirred solution of Fulvestrant (0.250 g) in dichloromethane (4 mL) were added diisopropylethylamine (0.11 mL) and triphosgene (0.062 g) at 0° C. and stirred the reaction mixture for 10 min at the same temperature. After 10 that 4-(pyrrolidin-1-yl)piperidine (0.099 g) was added in the reaction mixture at 0° C. and the resulting mixture was stirred at room temperature for 16 h. After completion of reaction on TLC, the reaction mass was diluted with water (4 mL) and extracted with dichloromethane (2×10 mL). The combined organic layer was dried over anhydrous sodium sulphate and concentrated under reduced pressure to give 0.8 g crude material, which was purified by flash chromatography using 8-9% methanol in dichloromethane. The desired fraction was concentrated under reduced pressure to obtained pure compound.

LCMS purity: 99.25% (m/z: 787.51, [M+1]⁺, 214 nm).

¹H NMR (400 MHz, DMSO-d₆): δ 7.27 (d, J=8.4 Hz, 1H), 6.82 (d, J=8.4 Hz, 1H), 6.78 (s, 1H), 4.50 (d, J=4.4 Hz, 1H), 4.02-3.88 (m, 2H), 3.57-3.52 (m, 1H), 3.10 (bs, 1H), 2.96 (bs, 1H), 2.88-2.62 (m, 7H), 2.48-2.27 (m, 7H), 1.94-1.79 (m, 6H), 1.69-1.48 (m, 9H), 1.38-1.16 (m, 21H), 0.90-0.85 (m, 1H), 0.67 (s, 3H)

### EXAMPLE-10

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-9-methyl-3,9-diazaspiro[5.5]undecane-3-carboxylate

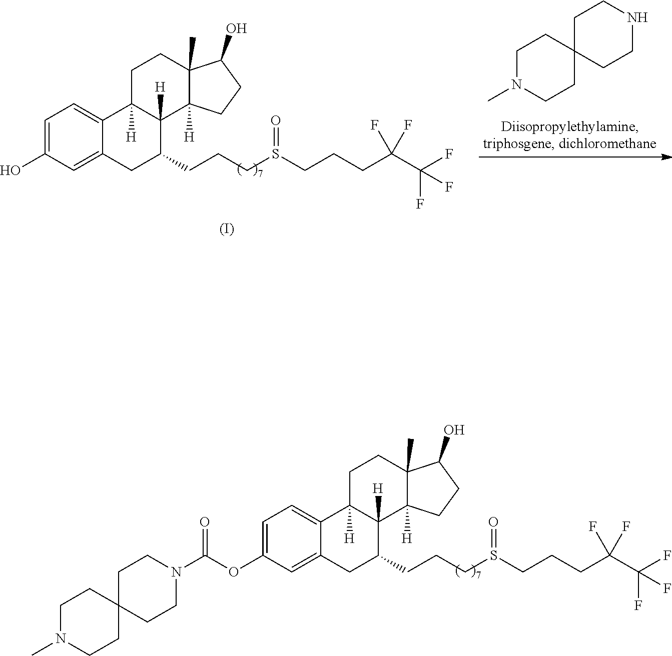

To a stirred solution of Fulvestrant (0.5 g) in dichloromethane (7 mL) were added diisopropylethylamine (0.21 mL) and triphosgene (0.122 g) at 0° C. and stirred the reaction mixture for 10 min at the same temperature. After 10 min, 3-methyl-3,9-diazaspiro[5.5]undecane (0.207 g) was added in the reaction mixture at 0° C. and the reaction mixture was stirred at room temperature for 16 h. After completion of reaction on TLC, the reaction mass was diluted with water (5 mL) and extracted with dichloromethane (3×40 mL). The combined organic layer was dried over anhydrous sodium sulphate and evaporated under reduced pressure to obtained crude material. The crude was purified by prep HPLC and the product fractions were lyophilized to give 0.060 g of (7R,8R,9S,13S,14S,17S)-17-hydroxy-13-methyl 7(9((4,4,5,5,5pentafluoropentyl)sulfinyl)nonyl)-7,8,9,11,12,13,14,15,16,17-decahydro-6H-cyclopenta[a]phenanthren-3-yl-9-methyl-3,9-diazaspiro[5.5]undecane-3-carboxylate as a white colour solid.

LCMS: 98.96% (m/z: 801.46, [M+1]⁺, 214 nm).

¹H NMR (400 MHz, DMSO-d₆): δ 7.24 (d, J=8.4 Hz, 1H), 6.81 (dd, J=2, 8.8 Hz, 1H), 6.76 (d, J=2 Hz, 1H), 4.49 (d, J=4.8 Hz, 1H), 3.53 (m, 3H), 3.39 (bs, 2H), 2.84-2.50 (m, 6H), 2.49-2.26 (m, 9H), 1.90-1.79 (m, 4H), 1.67-1.56 (m, 5H), 1.60-1.18 (m, 28 H), 1.17 (m, 1H), 0.67 (s, 3H).

### EXAMPLE-11

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 6-methyl-2,6-diazaspiro[3.3]heptane-2-carboxylate

### Step-1: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl(4-nitrophenyl) carbonate

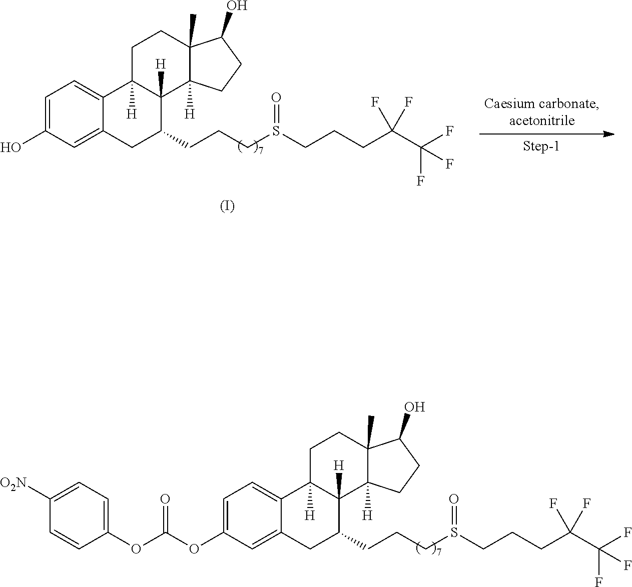

To a stirred solution of Fulvestrant (2.0 g) in acetonitrile (20 mL) were added caesium carbonate (3.24 g) and 4-nitrophenyl chloroformate (0.99 g) at room temperature and stirred the reaction mixture for 15 min at the same temperature. After completion of the reaction on TLC, the reaction mass was diluted with water (15 mL) and extracted with ethyl acetate (2×30 mL), combined organic layer was dried over anhydrous sodium sulphate, and concentrated the organics under reduced pressure to give 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (2.3 g crude, LCMS 60%), which was used for next step without further purification.

LCMS purity: 60.00%

### Step-2: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 6-methyl-2,6-diazaspiro[3.3]heptane-2-carboxylate

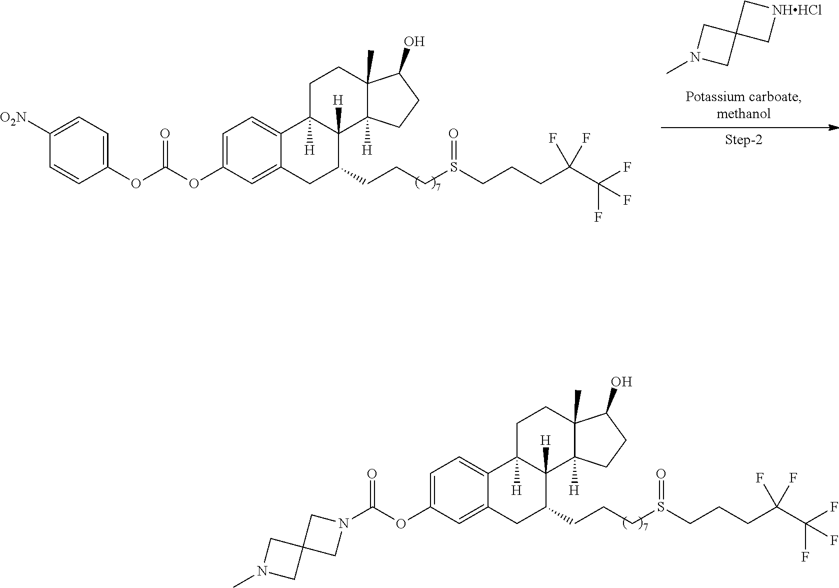

To a stirred solution of 2-methyl-2,6-diazaspiro[3.3]heptane hydrochloride (0.109 g) in acetonitrile (5 mL) was added potassium carbonate (0.93 g) and stirred at room temperature for 2 h. Then (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (0.500 g) was added and reaction mass was stirred at room temperature for 30 min. After completion of reaction on TLC, the reaction mass was diluted with ice water (15 mL) and extracted with ethyl acetate (2×30 mL). Combined the organic layer and dried over anhydrous sodium sulphate, then concentrated the organics under reduced pressure to give 0.450 g crude material, which was purified by Prep HPLC and the product fractions were lyophilized to give 0.150 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 6-methyl-2,6-diazaspiro[3.3]heptane-2-carboxylate as a white colour solid.

LCMS: 99.24% (m/z: 745.37, [M+1]⁺, 214 nm).

¹H NMR (400 MHz, DMSO-d₆): δ 7.24 (d, J=8.8 Hz, 1H), 6.81 (dd, J=2.4, 8.8 Hz, 1H), 6.75 (d, J=2.4 Hz, 1H), 4.50 (bs, 1H), 4.16 (s, 2H), 3.99 (s, 2H), 3.54 (t, J=8.8 Hz, 1H), 3.23 (s, 4H), 2.84-2.80 (m, 2H), 2.76-2.70 (m, 2H), 2.66-2.64 (m, 1H), 2.39-2.37 (m, 2H), 2.32-2.28 (m, 2H), 2.15 (s, 3H), 1.92-1.86 (m, 3H), 1.78 (m, 1H), 1.68 (m, 1H), 1.64 (m, 1H), 1.61-1.55 (m, 4H), 1.37-1.15 (m, 18H), 0.90-0.87 (m, 1H), 0.66 (s, 3H).

### EXAMPLE 12

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-(dimethylamino)ethyl)(methyl)carbamate

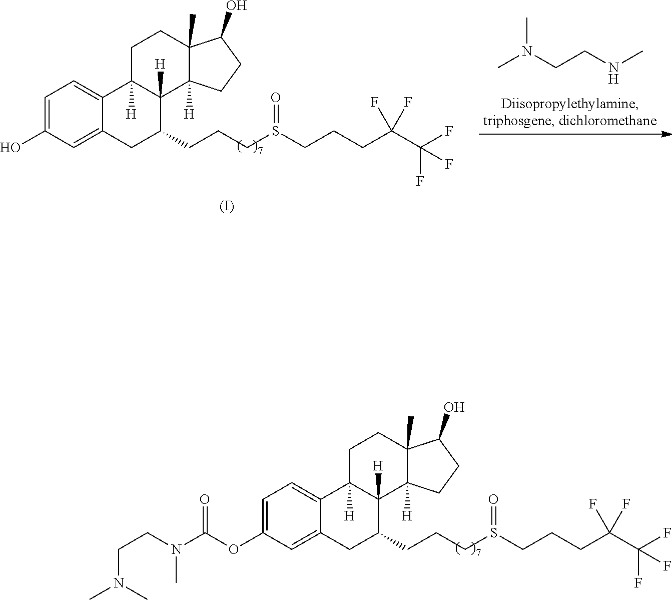

To a solution of Fulvestrant (0.5 g) in dichloromethane (5 mL) was added diisopropylethylamine (0.23 mL) followed by triphosgene (0.12 g) at 0° C. and stirred the reaction mass for 10 min. Then added N1,N1,N2-trimethylethane-1,2-diamine (0.29 g) and stirred the reaction mixture again at same temperature for 15 min and then at room temperature for 18 h. The progress of the reaction progress was monitored by TLC (mobile phase: 50% ethyl acetate in Heptane). After completion of the reaction by LCMS, diluted the reaction mixture with water (5 mL) and extracted with dichloromethane (2×25 mL), combined the organic layers and dried over anhydrous sodium sulphate. The organic layer was evaporated under reduced pressure to obtained crude material. The crude compound was purified by prep-HPLC to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-(dimethylamino)ethyl)(methyl)carbamate (0.04 g) as white solid.

LCMS: 98.0% (m/z: 735.45, [M+1]⁺, 214 nm).

¹H NMR (400 MHz, DMSO-d₆): δ 7.27 (d, J=8.4 Hz, 1H), 6.81-6.77 (m, 2H), 4.49 (d, J=4.8 Hz, 1H), 3.57-3.54 (m, 1H), 3.44-3.32 (m, 2H), 3.01-2.89 (m, 3H), 2.86-2.64 (m, 5H) 2.39-2.24 (m, 5H), 2.19-2.17 (m, 6H), 1.94-1.49 (m, 9H), 1.35-1.24 (m, 20H), 0.90 (bs, 1H), 0.67 (s, 3H).

### EXAMPLE 13

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(pyridin-4-yl)piperidine-1-carboxylate

### Step-1: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl(4-nitrophenyl) carbonate

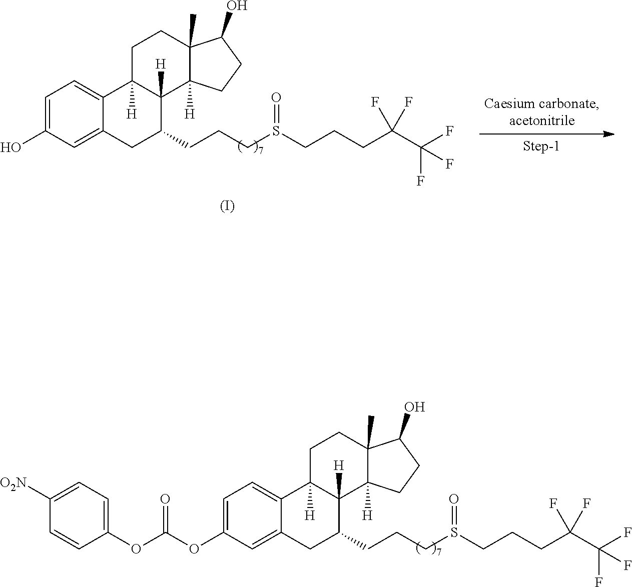

To a stirred solution of Fulvestrant (2.0 g) in acetonitrile (20 mL) were added caesium carbonate (3.24 g) and 4-nitrophenyl chloroformate (0.99 g) at room temperature and stirred the reaction mixture for 15 min at the same temperature. After completion of the reaction on TLC, the reaction mass was diluted with water (15 mL) and extracted with ethyl acetate (2×30 mL), combined organic layer was dried over anhydrous sodium sulphate, and concentrated the organics under reduced pressure to give 7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-nitrophenyl) carbonate (2.3 g crude, LCMS 60%), which was used for next step without further purification.

LCMS purity: 60.00%

### Step-2: Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(pyridin-4-yl)piperidine-1-carboxylate

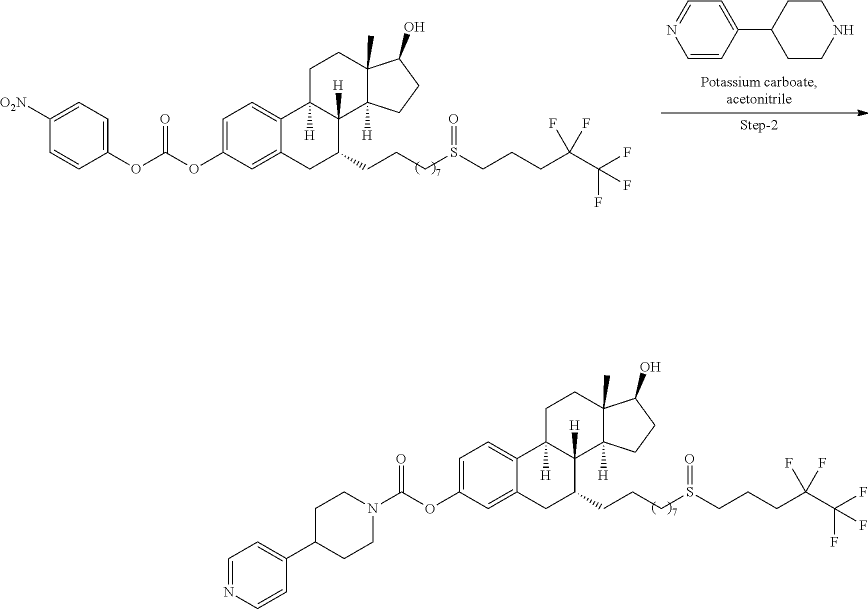

To a solution of 4-(piperidin-4-yl)pyridine (0.094 g) in acetonitrile (5 mL) was added potassium carbonate (0.134 g) followed by the addition of (7R,8R,9S,13S,14S,17S)-17-hydroxy-13-methyl-7-(9-((4,4,5,5,5-pentafluoropentyl)sulfinyl) nonyl)-7,8,9,11,12,13,14,15,16,17-decahydro-6H-cyclopenta[a]phenanthren-3-yl(4-nitrophenyl) carbonate (0.3 g). The reaction was then stirred at room temperature for 45 mins. The progress of the reaction was monitored by TLC (mobile phase: 70% ethyl acetate in hexane). After completion of the reaction, the mixture was extracted with ethyl acetate, dried the organic layer over anhydrous sodium sulphate and the organic layer was evaporated under reduced pressure to obtained crude material. The crude compound was purified by prep-HPLC to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(pyridin-4-yl)piperidine-1-carboxylate (0.07 g, 99%) as a white solid.

LCMS: 99.14% (m/z: 795.49, [M+1]⁺, 214 nm).

¹H NMR (400 MHz, DMSO-d₆): δ 8.49 (d, J=5.2 Hz, 2H), 7.32 (d, J=5.2 Hz, 2H), 7.27 (d, J=8.8 Hz, 1H), 6.85 (d, J=8.8 Hz, 1H), 6.81 (s, 1H), 4.50 (d, J=4.4 Hz, 1H), 4.24-4.16 (m, 2H), 3.56-3.55 (m, 1H), 3.10-2.95 (m, 2H), 2.88-2.64 (m, 7H), 2.37-2.28 (m, 3H), 1.94-1.80 (m, 6H), 1.69-1.50 (m, 8H), 1.48-1.23 (m, 18H) 0.91 (bs, 1H), 0.68 (s, 3H).

### EXAMPLE-14

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(dimethylamino)piperidine-1-carboxylate

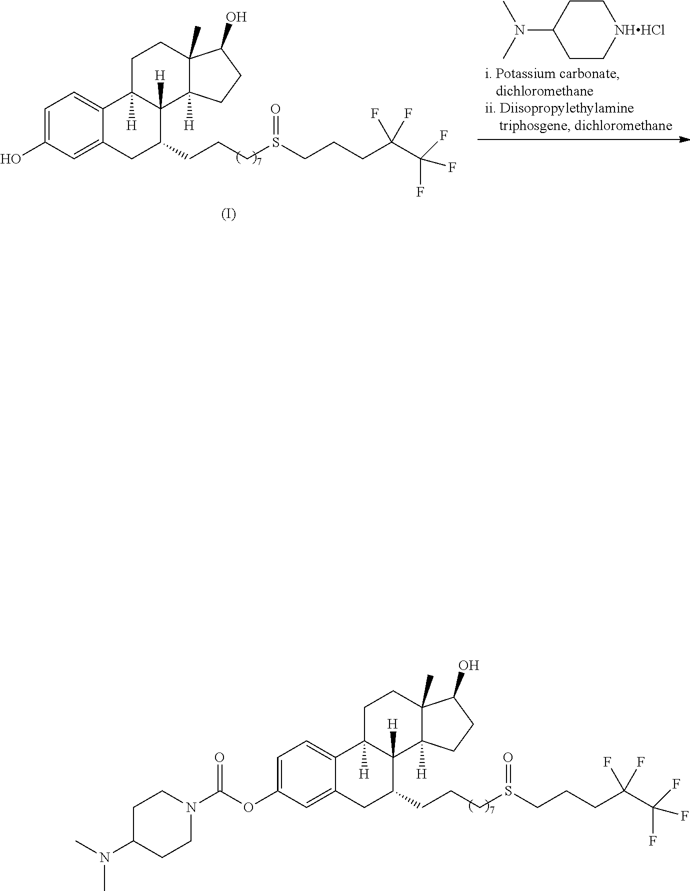

To a solution of N,N-dimethylpiperidin-4-amine.hydrochloride (0.081 g) in dichloromethane (2 mL) was added potassium carbonate (0.341 g) and stirred this reaction mixture-1 at room temperature for 1 h. In another vessel, Fulvestrant (0.2 g) was taken in dichloromethane (2 mL) and added diisopropylethylamine (0.086 mL) followed by triphosgene (0.049 g) at 0° C. under nitrogen atmosphere. The mixture was left at the same temperature for 15 mins and then reaction mixture-1 was added and continued stirring at room temperature for 18 h. The progress of the reaction was monitored by TLC (mobile phase: 70% ethyl acetate in heptane). After completion of the reaction, extracted with ethyl acetate, dried the organic layer over anhydrous sodium sulphate and the organic layer was evaporated under reduced pressure to obtained crude material. The crude compound was purified by flash column chromatography to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(dimethylamino)piperidine-1-carboxylate (0.04 g) as white solid.

LCMS: 99.04% (m/z: 761.77, [M+1]⁺, 214 nm).

¹H NMR (400 MHz, DMSO-d₆): δ 7.26 (d, J=8.8 Hz, 1H), 6.82 (d, J=8.4 Hz, 1H), 6.78 (s, 1H), 4.50 (d, J=4.4 Hz, 1H), 4.13-4.02 (m, 2H), 3.57-3.52 (m, 1H), 3.00 (bs, 1H), 2.94-2.81 (m, 3H), 2.76-2.61 (m, 4H), 2.40-2.30 (m, 9H), 1.94-1.79 (m, 6H), 1.69 (bs, 1H), 1.62-1.54 (m, 3H), 1.49-1.18 (m, 23H), 0.98-0.85 (m, 1H), 0.67 (s, 3H).

### EXAMPLE-15

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(1,4-oxazepan-4-yl)piperidine-1-carboxylate

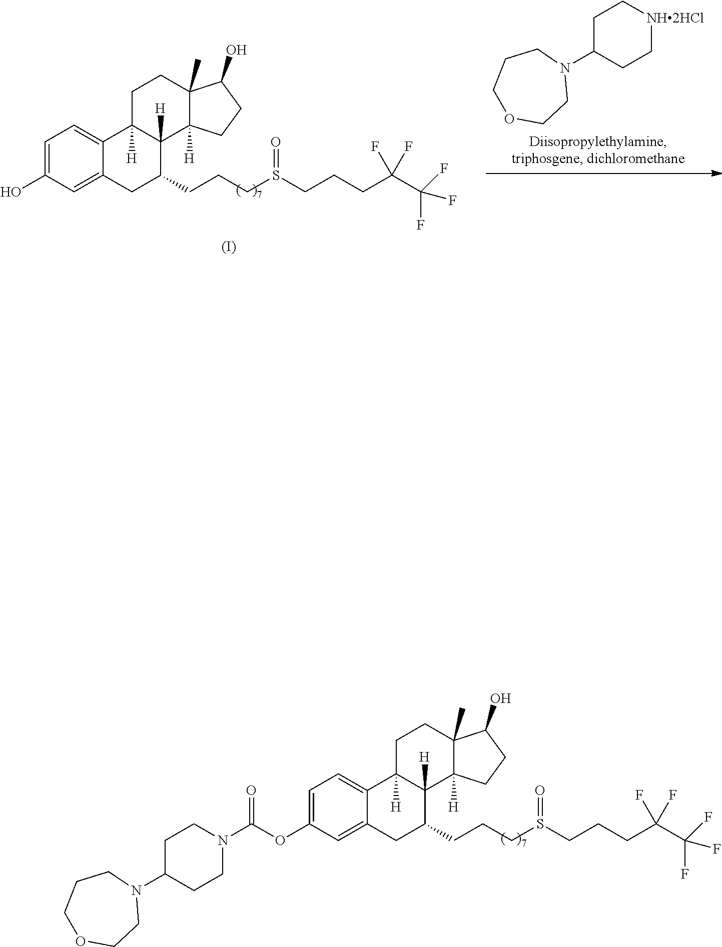

To a solution of 4-(piperidin-4-yl)-1,4-oxazepane (0.250 g) in dichloromethane (2 mL) was added triethylamine (0.341 g) and stirred this reaction mixture-1 at room temperature for 2 h. In another vessel, Fulvestrant (0.27 g) was taken in dichloromethane (5 mL) was added diisopropylethylamine (0.12 mL) followed by triphosgene (0.067 g) at 0° C. and the reaction mixture was stirred at the same temperature for 15 mins. To this mixture was added reaction mixture-1 and stirred at room temperature for 1 h. The progress of the reaction was monitored by TLC (mobile phase: 5% methanol in dichloromethane). After completion of the reaction, the reaction mass was extracted with dichloromethane, dried the organic layer over anhydrous sodium sulphate and the organic layer was evaporated under reduced pressure to obtained crude material. The crude compound was purified by prep-HPLC to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-4-(1,4-oxazepan-4-yl)piperidine-1-carboxylate (0.065 g) as white solid.

LCMS: 99.64% (m/z: 817.56, [M+1]⁺, 214 nm).

¹H NMR (400 MHz, DMSO-d₆), TFA salt: δ 7.28 (d, J=8.8 Hz, 1H), 6.84 (dd, J=2.4, 8.4 Hz, 1H), 6.79 (s, J=2.4 Hz, 1H), 4.49 (bs, 1H), 4.15-4.16 (m, 2H), 3.86-3.77 (m, 3H), 3.71-3.53 (m, 3H), 3.35-3.25 (m, 3H), 3.02-2.81 (m, 4H), 2.76-2.61 (m, 5H), 2.44-2.25 (m, 4H), 2.06-2.03 (m, 4H), 1,94-1.85 (m, 3H), 1.83-1.80 (m, 1H), 1.72-1.54 (m, 6H), 1.50-1.16 (m, 19H), 0.92-0.87 (m, 1H), 0.67 (s, 3H).

### EXAMPLE-16

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 3-oxa-9-azaspiro[5.5]undecane-9-carboxylate

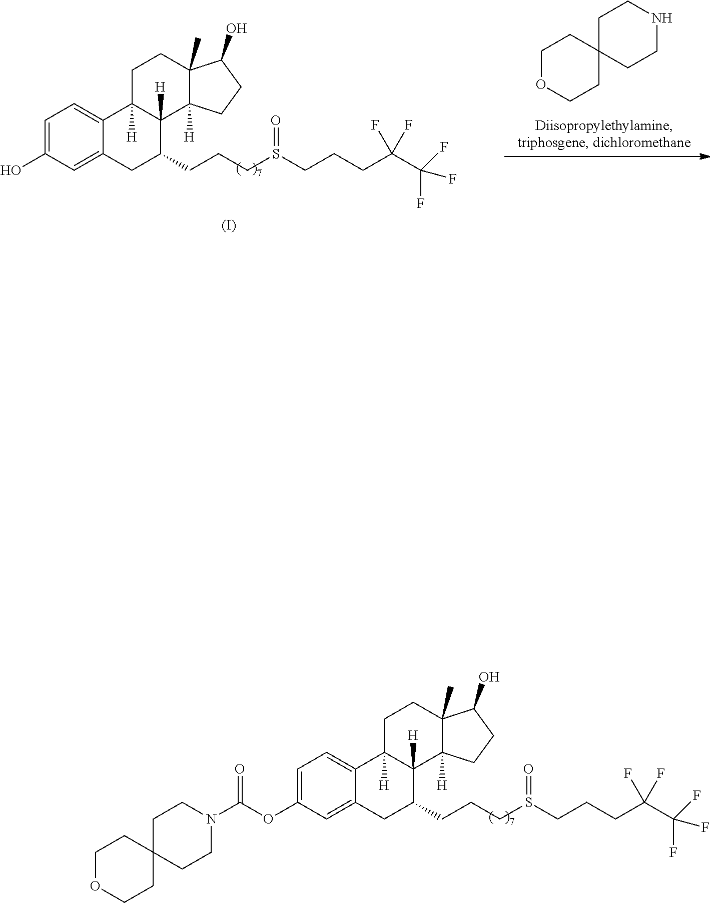

To a stirred solution of Fulvestrant (0.300 g) in dichloromethane (10 mL) were added diisopropylethylamine (0.12 mL) and triphosgene (0.07 g) at 0° C. and stirred the reaction mixture for 10 min at the same temperature. After 10 min, 3-oxa-9-azaspiro[5.5]undecane (0.15 g) was added in the reaction mixture at 0° C. and the reaction mixture was stirred at room temperature for 16 h. After completion of reaction on TLC, the reaction mass was diluted with water (25 mL) and extracted with dichloromethane (2×25 mL). The combined organic layer was dried over anhydrous sodium sulphate and concentrated under reduced pressure to give 0.5 g crude material (crude LCMS purity 60%). The crude was purified by prep HPLC to give 0.100 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl-3-oxa-9-azaspiro[5.5]undecane-9-carboxylate as a white colour solid. (Yield:100 mg, 32.6%)

LCMS: 94.37% (m/z: 788.68, [M+1]⁺, 214 nm).

¹H NMR (400 MHz, DMSO-d₆) δ 7.26 (d, J=8.8 Hz, 1H), 6.81 (d, J=8.4 Hz, 1H), 6.77 (s, 1H), 4.50 (bs, 1H), 3.58-3.55 (m, 7H), 3.40 (bs, 2H), 2.88-2.81 (m, 2H), 2.76-2.60 (m, 4H), 2.45-2.24 (m, 4H), 1.94-1.87 (m, 3H), 1.82-1.80 (m, 1H), 1.71-1.69 (m, 1H), 1.64-1.45 (m, 11H), 1.38-1.18 (m, 19H), 0.90-0.85 (m, 1H), 0.67 (s, 3H).

### EXAMPLE-17

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-(1-methylpiperidin-4-yl)phenyl)carbamate

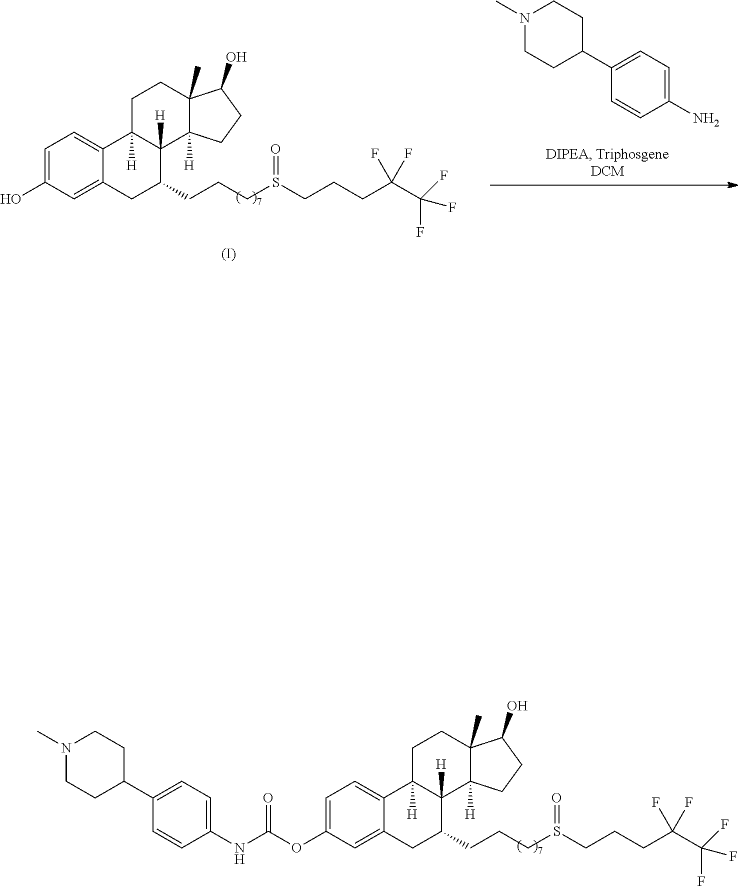

To a solution of Fulvestrant (0.5 g) in DCM (8 mL) were added DIPEA (0.21 mL) and triphosgene (0.122 g) at 0° C. and stirred the reaction mixture for 10 min at the same temperature. After 10 min, 4-(1-methylpiperidin-4-yl)aniline (0.235 g) was added in the reaction mixture at 0° C. and the reaction mixture was stirred at room temperature for 16 h. After completion of reaction on TLC, the reaction mixture was diluted with water (5 mL) and extracted with DCM (2×15 mL). The combined organic layer was dried over anhydrous sodium sulfate and concentrated under reduced pressure to obtain 0.7 g of crude material which was further purified by reverse-phase chromatography to obtain (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-(1-methylpiperidin-4-yl)phenyl)carbamate as a light brown solid.

LCMS purity: 88%

### EXAMPLE-18

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-morpholinophenyl)carbamate

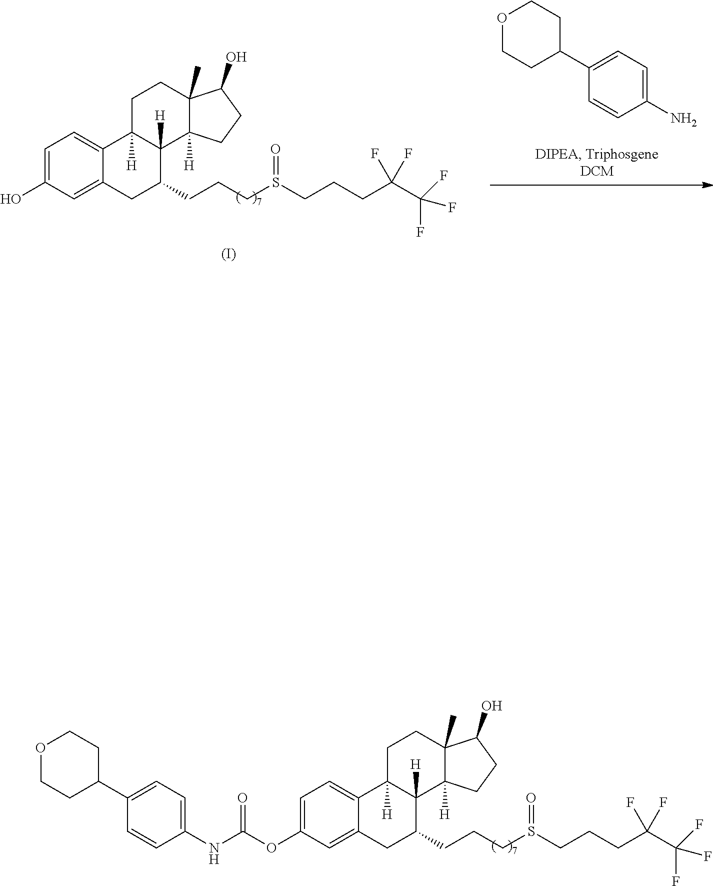

To a solution of Fulvestrant (0.5 g) in DCM (8 mL) were added DIPEA (0.21 mL) and triphosgene (0.122 g) at 0° C. and stirred the reaction mixture for 10 min at the same temperature. After 10 min, 4-morpholinoaniline (0.220 g) was added in the reaction mixture at 0° C. and the reaction mixture was stirred at room temperature for 16 h. After completion of reaction on TLC, the reaction mixture was diluted with water (5 mL) and extracted with DCM (2×15 mL). The combined organic layer was dried over anhydrous sodium sulfate and concentrated under reduced pressure to obtain 0.7 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (4-morpholinophenyl)carbamate as a light yellow gum.

LCMS purity: 66%

### EXAMPLE-19

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-(dimethylamino)ethyl)carbamate

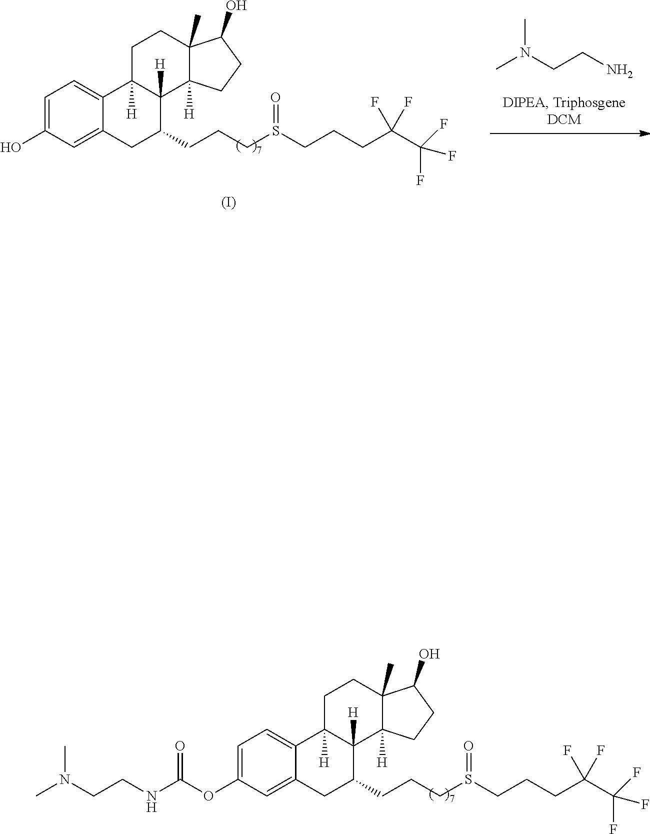

To a solution of Fulvestrant (0.5 g) in DCM (8 mL) were added DIPEA (0.21 mL) and triphosgene (0.122 g) at 0° C. and stirred the reaction mixture for 10 min at the same temperature. After 10 min, N,N-dimethylethane-1,2-diamine (0.108 g) was added in the reaction mixture at 0° C. and the reaction mixture was stirred at room temperature for 16 h. After completion of reaction on TLC, the reaction mixture was diluted with water (5 mL) and extracted with DCM (2×15 mL). The combined organic layer was dried over anhydrous sodium sulfate and concentrated under reduced pressure to obtain 0.5 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl (2-(dimethylamino)ethyl)carbamate as a light brown solid.

LCMS purity: 22%

### EXAMPLE-20

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-morpholinopiperidine-1-carboxylate

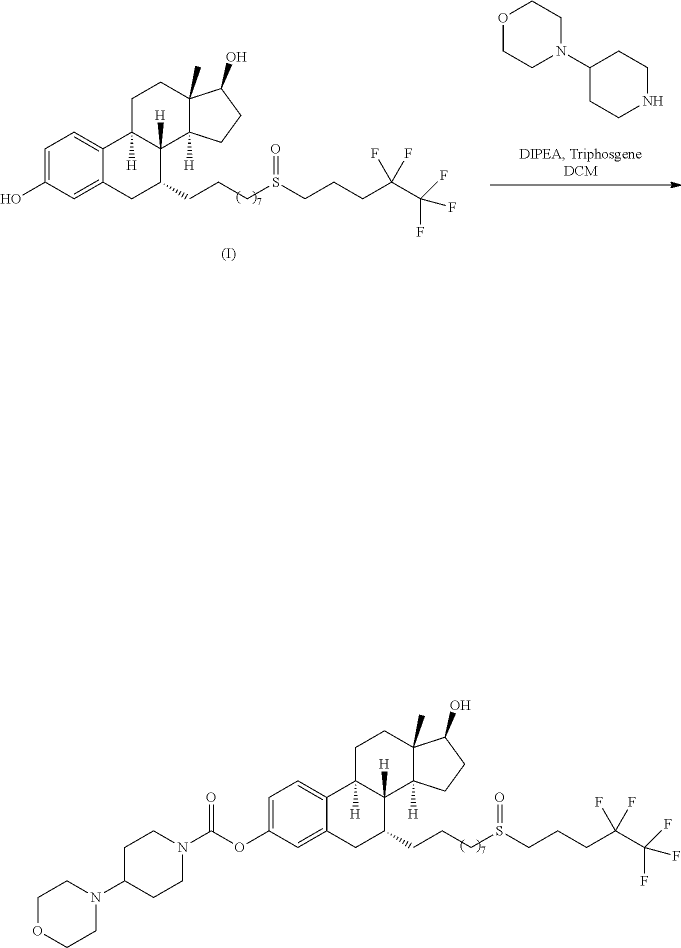

To a solution of Fulvestrant (0.5 g) in DCM (8 mL) were added DIPEA (0.21 mL) and triphosgene (0.122 g) at 0° C. and stirred the reaction mixture for 10 min at the same temperature. After 10 min, 4-(piperidin-4-yl)morpholine (0.209 g) was added in the reaction mixture at 0° C. and the reaction mixture was stirred at room temperature for 16 h. After completion of reaction on TLC, the reaction mixture was diluted with water (5 mL) and extracted with DCM (2×15 mL). The combined organic layer was dried over anhydrous sodium sulfate and concentrated under reduced pressure to obtain 0.56 g of (7R,8R,9S,13S,14S,17S)-17-hydroxy-13-methyl-7-(9-((4,4,5,5,5-pentafluoropentyl)sulfinyl)nonyl)-7,8,9,11,12,13 ,14,15,16,17-decahydro-6H-cyclopenta[a]phenanthren-3-yl 4-morpholinopiperidine-1-carboxylate as a light brown solid.

LCMS purity: 48%

### EXAMPLE-21

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(4-methylpiperazin-1-yl)piperidine-1-carboxylate

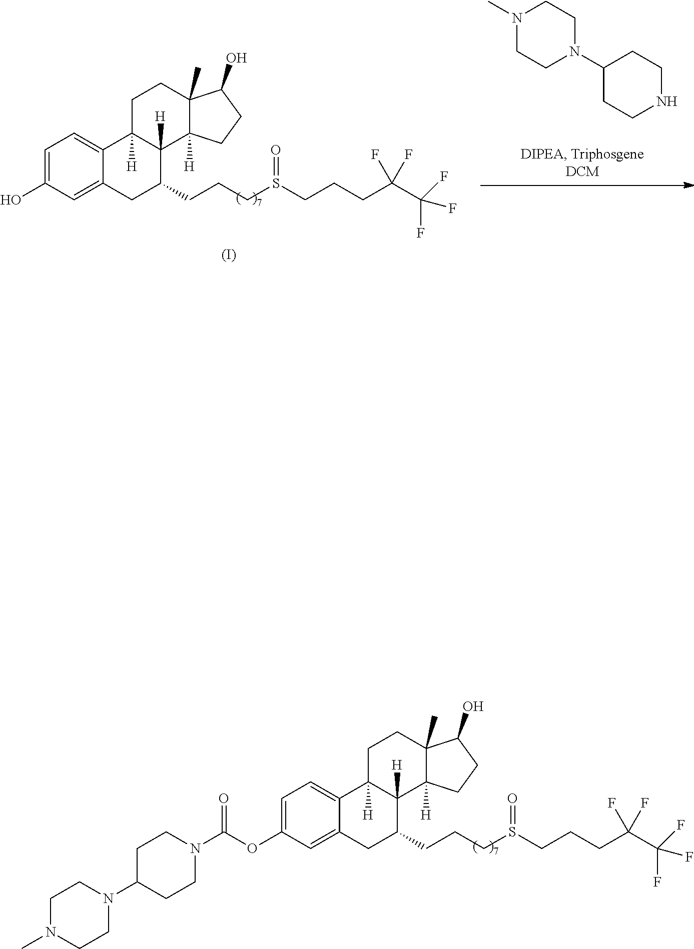

To a solution of Fulvestrant (0.5 g) in DCM (8 mL) were added DIPEA (0.21 mL) and triphosgene (0.122 g) at 0° C. and stirred the reaction mixture for 10 min at the same temperature. After 10 min, 1-methyl-4-(piperidin-4-yl)piperazine (0.225 g) was added in the reaction mixture at 0° C. and the reaction mixture was stirred at room temperature for 16 h. After completion of reaction on TLC, the reaction mixture was diluted with water (5 mL) and extracted with DCM (2×15 mL). The combined organic layer was dried over anhydrous sodium sulfate and concentrated under reduced pressure to obtain 0.1 g of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 4-(4-methylpiperazin-1-yl)piperidine-1-carboxylate as a light brown solid.

LCMS purity: 65%

### EXAMPLE-22

### Preparation of (7R,8R,9S,13S,14S,17S)-13-methyl-7-(9-((4,4,5,5,5-pentafluoropentyl)sulfinyl)nonyl)-17-(phosphonooxy)-7,8,9,11,12,13,14,15,16,17-decahydro-6H-cyclopenta[a]phenanthren-3-yl [1,4′-bipiperidine]-1′-carboxylate

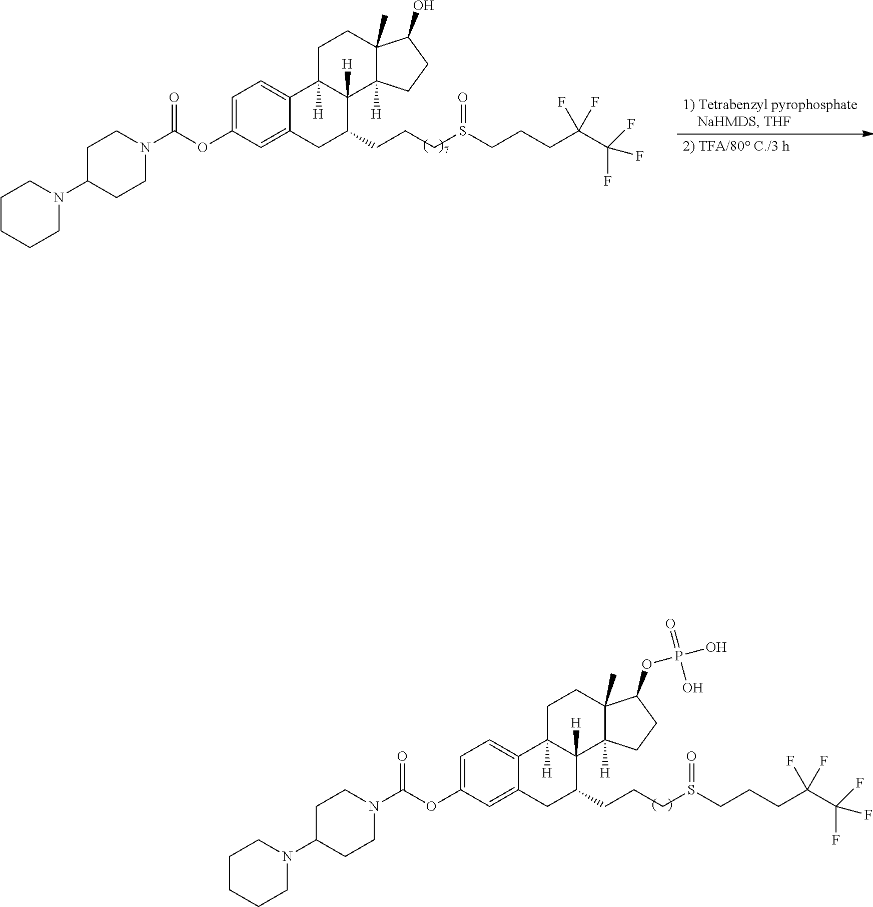

Step-1: To a solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate (0.500 g) in THF (5 mL) was added NaHMDS (1.25 mL) at 0° C. The resulting reaction mixture was stirred at the same temperature for 30 min. Then added tetrabenzyl pyrophosphate (0.671 g) at room temperature. Then reaction mixture was stirred at rt for 16 h. After completion of reaction by LCMS, the reaction mixture was diluted with water and extracted with EtOAc. The combined organic layer was dried over anhydrous sodium sulfate and concentrated under reduced pressure to obtain (7R,8R,9S,13S,14S,17S)-17-((bis(benzyloxy)phosphoryl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate (550 mg 21% pure by LCMS) which was used in next step without further purification.

Step-2: To a solution of (7R,8R,9S,13S,14S,17S)-17-((bis(benzyloxy)phosphoryl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate (step-1 product 0.550 g) in TFA (6 mL) was stirred at 80° C. for 3h. After completion of reaction by LCMS, reaction mixture was cooled to rt and concentrated under reduced pressure to obtained the crude material (450 mg, 21% pure by LCMS) which was purified by prep HPLC and obtained 23 mg of (7R,8R,9S,13S,14S,17S)-13-methyl-7-(9-((4,4,5,5,5-pentafluoropentyl)sulfinyl)nonyl)(phosphonooxy)-7,8,9,11,12,13,14,15,16,17-decahydro-6H-cyclopenta[a]phenanthren-3-yl [1,4′-bipiperidine]-1′-carboxylate

LCMS purity: 97%

### EXAMPLE-23

### Preparation of (7R,8R,9S,13S,14S,17S)-17-((D-valyl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate

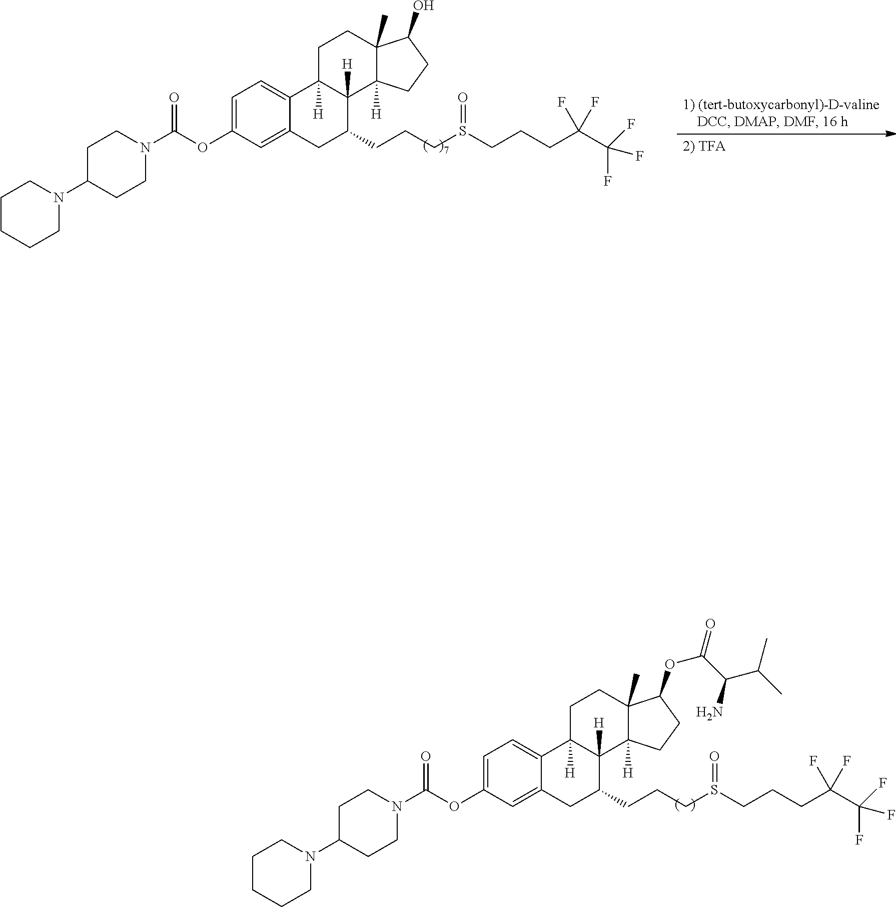

Step-1: To a stirred solution of (7R,8R,9S,13S,14S,17S)-17-hydroxy-13-methyl-7-(94(4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate (0.5 g) in DMF (10 mL) were added DCC (0.386 g) and DMAP (0.045 g) at room temperature. After 20 minutes, (tert-butoxycarbonyl)-D-valine (0.244 g) was added and the resulting mixture was stirred at room temperature for 16 h. After completion of reaction by LCMS, the reaction mixture was diluted with water and extracted with EtOAc. The combined organic layer was dried over anhydrous sodium sulfate and concentrated under reduced pressure to obtain (7R,8R,9S,13S,14S,17S)-17-(((tert-butoxycarbonyl)-D-valyl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate (0.8 g 47% pure by LCMS) which was used next step without further purification.

Step-2: To a solution of (7R,8R,9S,13S,14S,17S)-17-(((tert-butoxycarbonyl)-D-valyl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate (Step-1 product 0.8 g) in DCM (50 mL) was added TFA (10 mL) at 0° C. The reaction mixture was stirred at room temperature for 16 h. After completion of the reaction (monitored by LCMS), the reaction mixture was concentrated under reduced pressure at 25-28° C. to obtain 0.7 g of (7R,8R,9S,13 S,14S,17S)-17-((D-valyl)oxy)-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl [1,4′-bipiperidine]-1′-carboxylate as a brown sticky oil.

LCMS purity: 49% (0.7 g, 49% pure by LCMS)

### EXAMPLE-24

### Preparation of 3-(((7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl)oxy)propane-1,2-diol

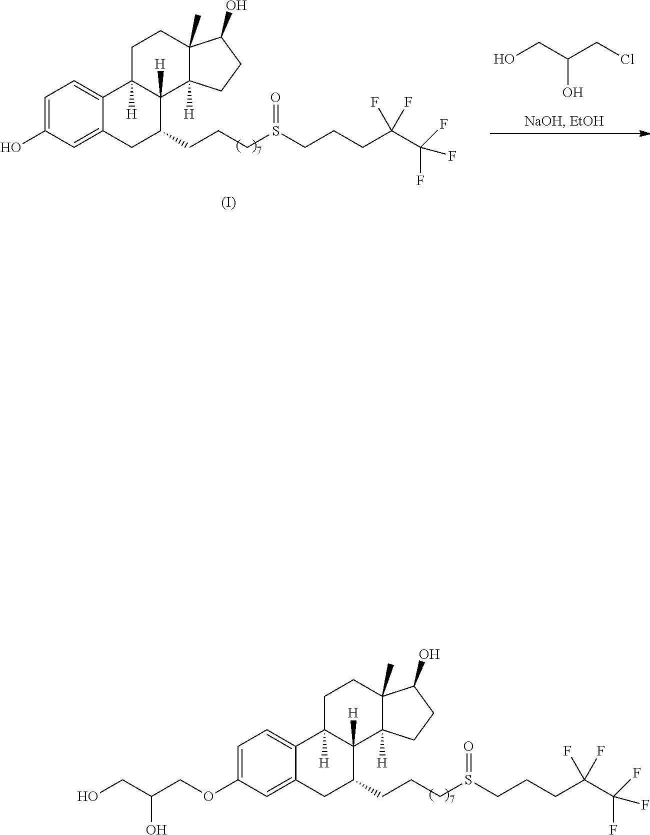

To a solution of Fulvestrant (0.150 g) in ethanol (3 mL) was added NaOH solution (0.014 gin 0.8 mL water) and the resulting solution was stirred at 80° C. for 30 min. To the above reaction mixture was then added 3-chloropropane-1,2-diol (0.040 g) and resulting suspension was stirred at 80° C. for 16 h. After completion of reaction on TLC, the reaction mass was cooled to room temperature and diluted with water (10 mL). The reaction mass was extracted with DCM (2×25 mL), the organic layer was washed with brine (2×10 mL), dried over anhydrous sodium sulfate and concentrated under reduced pressure to obtain the crude material which was further purified by preparative HPLC to afford 0.083 g of 3-(((7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl)oxy)propane-1,2-diol as a white semisolid (purity 98.5% by LCMS).

1H-NMR (DMSO): δ 7.159 (d, 1H), 6.662 (dd, 1H), 6.590 (d, 1H), 4.888 (d, 1H), 4.626 (t, 1H), 4.494 (d, 1H), 3.919-3.884 (m, 1H), 3.815-3.724 (m, 2H), 3.564-3.512 (m, 1H), 3.415 (t, 2H), 2.859-2.688 (m, 5H), 2.641-2.624 (m, 1H), 2.390-2.327 (m, 2H), 2.306-2.212 (m, 2H), 1.942-1.865 (m, 3H), 1.807-1.777 (m, 1H), 1.670-1.472 (m, 5H), 1.367-1.166 (m, 18H), 0.937-0.904 (m, 1H), 0.663 (s, 3H)

### EXAMPLE-25

### Preparation of 2-hydroxy-4-(((7R,8R,9S,13S,14S,17S)-17-hydroxy-13-methyl-7-(94(4,4,5,5,5-pentafluoropentyl)sulfinyl) nonyl)-7,8,9,11,12,13,14,15,16,17-decahydro-6H-cyclopenta[a]phenanthren-3-yl)oxy)-4-oxobutanoic acid

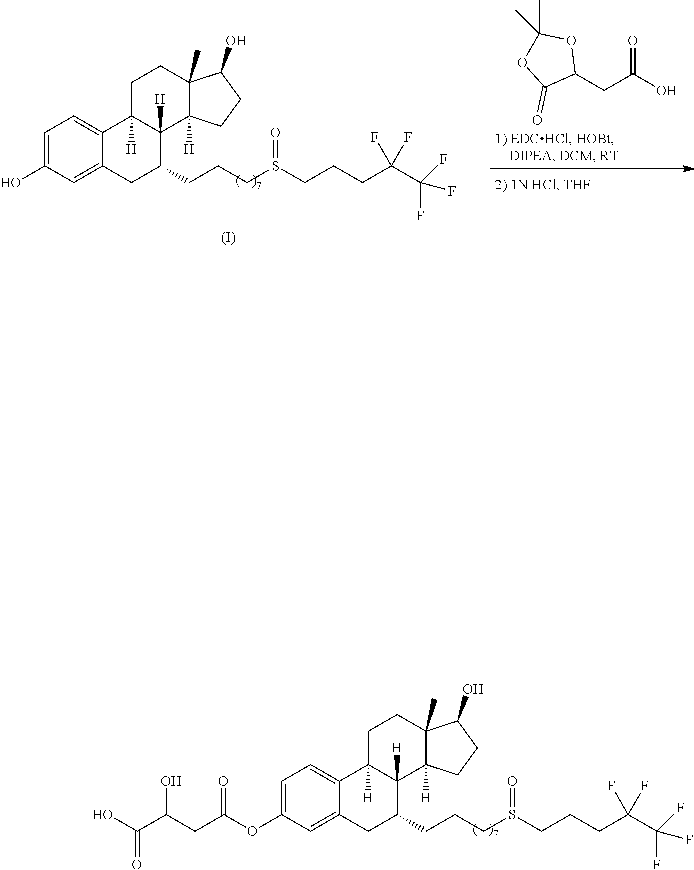

Step-1: To a stirred solution of Fulvestrant (0.7 g) and 2-(2,2-dimethyl-5-oxo-1,3-dioxolan-4-yl)acetic acid (0.201 g) in DCM were added DIPEA (1 mL), EDC.HCl (0.661 g) and HOBt (0.265 g) followed by DMAP (0.028 g) and the resulting reaction mixture was vigorously stirred at room temperature for 16 h. After completion of the reaction on TLC, the reaction mixture was diluted with water and extracted with ethyl acetate, dried the organic layer over anhydrous sodium sulfate and concentrated under reduced pressure to obtain the (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl 2-(2,2-dimethyl-5-oxo-1,3-dioxolan-4-yl)acetate (0.7 g, LCMS purity 49%) as viscous oil, which was used without purification for the next step.

Step-2: To a stirred solution of (7R,8R,9S,13S,14S,17S)-17-hydroxy-13-methyl-7-(9-((4,4,5,5,5-pentafluoropentyl) sulfinyl)nonyl)-7,8,9,11,12,13,14,15,16,17-decahydro-6H-cyclopenta[a]phenanthren-3-yl 2-(2,2-dimethyl-5-oxo-1,3-dioxolan-4-yl)acetate (obtained from step-1, 0.7 g) in THF (7 mL) was added 2N HCl (7 mL) at 0° C. and the reaction mixture was then stirred at 70° C. for 6 h. After completion of the reaction by TLC, the reaction mixture was diluted with water and extracted with ethyl acetate (2×25 mL), dried the organic layer over anhydrous sodium sulfate and concentrated under reduced pressure to obtain the crude material which was purified by prep HPLC to give 0.031 g of 2-hydroxy-4-(((7R,8R,9S,13S,14S,17S)-17-hydroxy-13-methyl-7-(9-((4,4,5,5,5-pentafluoropentyl)sulfinyl) nonyl)-7,8,9,11,12,13,14,15,16,17-decahydro-6H-cyclopentalalphenanthren-3-yl)oxy)-4-oxobutanoic acid as a white solid.

1H-NMR (DMSO): δ 12.112 (br s, 1H), 7.044 (d, 1H), 6.495 (dd, 1H), 6.412 (d, 1H), 4.556-4.141 (m, 3H), 3.557-3.505 (m, 1H), 2.903-2.808 (m, 2H), 2.783-2.710 (m, 3H), 2.695-2.576 (m, 3H), 2.439-2.371 (m, 2H), 2.310-2.317 (m, 2H), 1.944-1.866 (m, 3H), 1.826-1.767 (m, 1H), 1.723-1.438 (m, 5H), 1.382-1.152 (m, 18H), 0.921-0.905 (m, 1H), 0.674-0.659 (m, 3H)

### EXAMPLE-26

### Preparation of tetraethyl ((7R,8R,9S,13S,14S,17S)-13-methyl-7-(9-((4,4,5,5,5-pentafluoropentyl)sulfinyl)nonyl)-7,8,9,11,12,13,14,15,16,17-decahydro-6H-cyclopenta[a]phenanthrene-3,17-diyl) bis(phosphate)

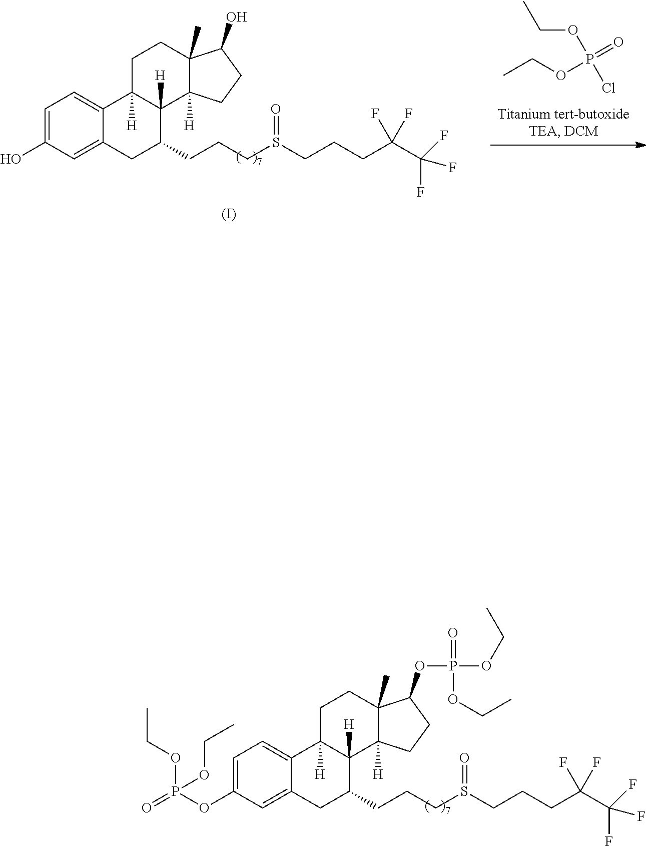

To a solution of Fulvestrant (0.250 g) in DCM (3 mL) were added TEA (0.3 mL) and titanium tertiary butoxide (0.068 g) and diethyl phosphorochloridate (0.354 g) and the resulting solution was stirred at rt for 16 h. After completion of reaction on TLC, the reaction mixture was diluted with water (10 mL), extracted with DCM (2×25 mL) and the organic layer was washed with brine (2×10 mL), dried over anhydrous sodium sulfate and concentrated under reduced pressure to obtained the crude material which was purified by prep HPLC to obtained tetraethyl ((7R,8R,9S,13S,14S,17S)-13-methyl-7-(94(4,4,5,5,5-pentafluoropentyl)sulfinyl)nonyl)-7,8,9,11,12,13,14,15,16,17-decahydro-6H-cyclopenta[a]phenanthrene-3,17-diyl) bis(phosphate) 0.092 g as a brown sticky liquid. (purity 95.6% by LCMS).

1H-NMR (DMSO): δ 7.299 (d, 1H), 6.941-6.919 (m, 1H), 6.878 (s, 1H), 4.262-4.203 (m, 1H), 4.166-4.091 (m, 4H), 4.045-3.958 (m, 4H), 2.861-2.809 (m, 2H), 2.747-2.611 (m, 4H), 2.421-2.300 (m, 3H), 2.114-2.082 (m, 1H), 1.942-1.884 (m, 2H), 1.855-1.832 (m, 1H), 1.720-1.693 (m, 1H), 1.621-1.566 (m, 5H), 1.491-1.334 (m, 9H), 1.271-1.228 (m, 20H), 1.169 (br s, 1H), 0.873 (br s, 1H), 0.771 (s, 3H)

### EXAMPLE-27

### Preparation of (7R,8R,9S,13S,14S,17S)-17-hydroxy-13-methyl-7-(9-((4,4,5,5,5-pentafluoropentyl)sulfinyl)nonyl)-7,8,9,11,12,13, 14,15,16,17-decahydro-6H-cyclopenta[a]phenanthren-3-yl dihydrogen phosphate, di(2S,3R,4R,5R)-2,3,4,5,6-pentahydroxy-N-methylhexan-1-aminium salt (dimeglumine salt)

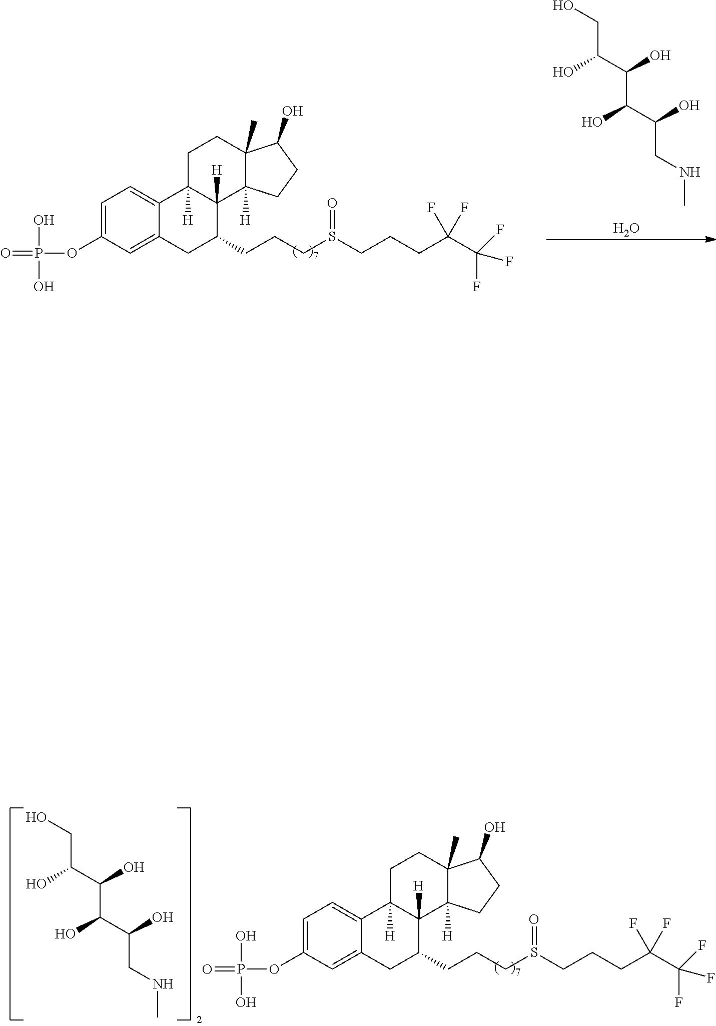

A solution of (7R,8R,9S,13S,14S,17S)-17-hydroxy-13-methyl-7-(94(4,4,5,5,5-pentafluoropentyl)sulfinyl)nonyl)-7,8,9,11,12,13,14,15,16,17-decahydro-6H-cyclopenta[a]phenanthren-3-yl dihydrogen phosphate (0.06 g) and (2R,3R,4R,5S)-6-(methylamino)hexane-1,2,3,4,5-pentaol (0.034 g) in water was stirred at room temperature for 12 h. The reaction mixture was lyophilized to afford (7R,8R,9S,13S,14S,17S)-17-hydroxy-13-methyl-7-(94(4,4,5,5,5-pentafluoropentyl)sulfinyl)nonyl)-7,8,9,11,12,13, 14,15,16,17-decahydro-6H-cyclopenta[a]phenanthren-3-yl dihydrogen phosphate, di(2S,3R,4R,5R)-2,3,4,5,6-pentahydroxy-N-methylhexan-1-aminium salt as a white solid (yield: 0.095 g, purity 96.12% by LCMS).

1H-NMR (DMSO): δ 7.064 (d, 1H), 6.818-6.796 (m, 2H), 4.891 (br s, 12H), 4.490 (br s, 1H), 3.710 (q, 2H), 3.645-3.632 (m, 2H), 3.604-3.568 (m, 2H), 3.537-3.517 (m, 1H), 3.498-3.454 (m, 3H), 3.413-3.325 (m, 6H), 2.868-2.815 (m, 2H), 2.767-2.696 (m, 3H), 2.642-2.599 (m, 4H), 2.393-2.371 (m, 2H), 2.323 (s, 6H), 2.291-2.184 (m, 2H), 1.941-1.883 (m, 3H), 1.865-1.809 (m, 1H), 1.658-1.486 (m, 5H), 1.370-1.167 (m, 18H), 0.924-0.890 (m, 1H), 0.665 (s, 3H)

### EXAMPLE-28

### Preparation of (7R,8R,9S,13S,14S,17S)-17-hydroxy-13-methyl-7-(9-((4,4,5,5,5-pentafluoropentyl)sulfinyl)nonyl)-7,8,9,11,12, 13,14,15,16,17-decahydro-6H-cyclopenta[a]phenanthren-3-yl dihydrogen phosphate di(2-amino-2-(hydroxymethyl)propane-1,3-diol)

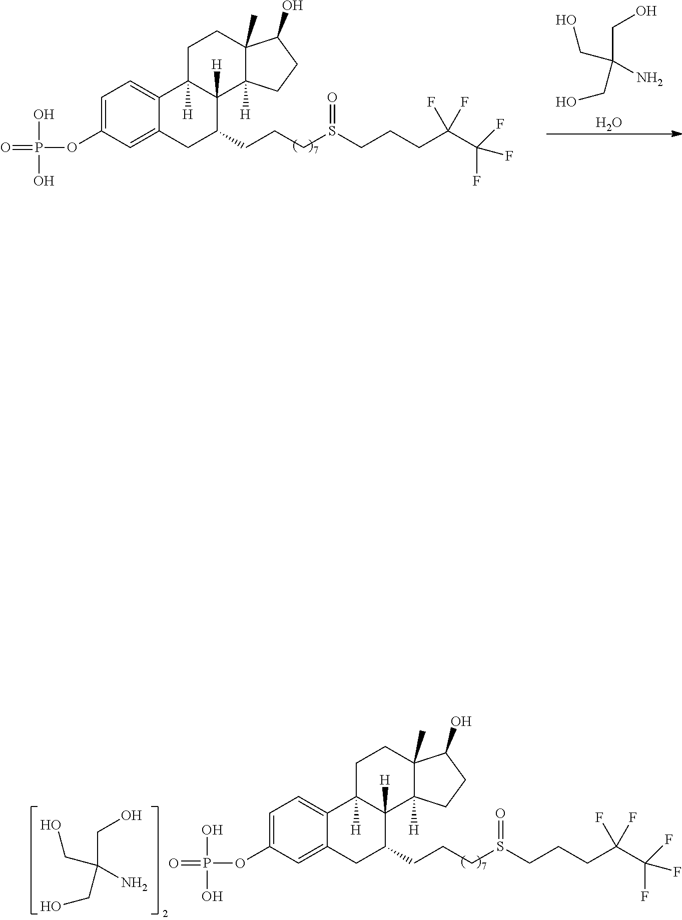

A solution of (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl dihydrogen phosphate (0.06 g) and 2-amino-2-(hydroxymethyl)propane-1,3-diol (0.021 g) in water was stirred at room temperature for 12 h. The reaction mixture was lyophilized to afford (7R,8R,9S,13S,14S,17S)-17-hydroxy-13-methyl-7-(9-((4,4,5,5,5-pentafluoropentyl)sulfinyl)nonyl)-7,8,9,11,12,13,14,15,16,17-decahydro-6H-cyclopenta[a]phenanthren-3-yl dihydrogen phosphate di(2-amino-2-(hydroxy methyl)propane-1,3-diol) as a white solid (yield: 0.077 g, purity 96.67% by LCMS).

1H-NMR (DMSO): δ 7.059 (d, 1H), 6.822-6.790 (m, 2H), 4.483 (br s, 9H), 3.538 (t, 2H), 3.281 (s, 12H), 2.868-2.598 (m, 7H), 2.391-2.182 (m, 5H), 1.943-1.866 (m, 3H), 1.809-1.777 (m, 1H), 1.682-1.660 (m, 1H), 1.619-1.486 (m, 4H), 1.369-1.168 (m, 19H), 0.936-0.918 (m, 1H), 0.665 (s, 3H).

1. A compound of formula I: 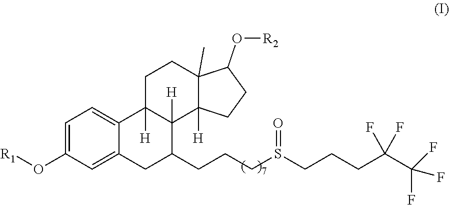
wherein
R₁ is 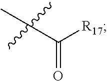
wherein R₁₇ is selected from NH₂, NHR₁₈, or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl and;
R₂ is selected independently from group comprising of:
i) 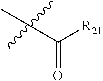
wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl;
ii) 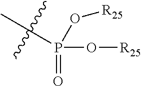
wherein each R₂₅ is selected independently from hydrogen; optionally substituted alkyl, aryl, or heteroaryl;
iii) 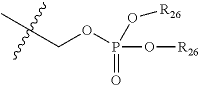
wherein each R₂₆ is selected independently from hydrogen; optionally substituted alkyl, cycloalkyl, heterocycloalkyl, aryl, or heteroaryl;
iv) amino acids, wherein the amino acid is linked via ester linkage at the point of attachment;
enantiomers, diastereomers, racemates, pharmaceutically acceptable salts or solvates thereof.

2. The compound of formula I according to claim 1, wherein R₂₁ is selected from optionally substituted alkyl, alkenyl, alkynyl, cycloalkyl, aryl, heteroaryl; wherein one or more substitution is selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate.

3. The compound of formula II according to claim 1: 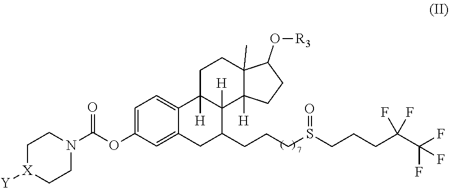
wherein X is selected from O, C, N; and
Y is selected from optionally substituted aryl, heteroaryl, heterocycloalkyl, or cycloalkyl; and
R₃ is selected independently from group comprising of:
i) 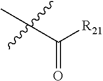
wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, alkoxycarbonyl, alkenyl, alkynyl, alkylthio and arylcarbonyl;
ii) 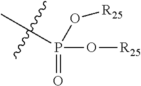
wherein each R₂₅ is selected independently from hydrogen; optionally substituted alkyl, aryl, or heteroaryl;
iii) 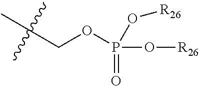
wherein each R₂₆ is selected independently from hydrogen; optionally substituted alkyl, cycloalkyl, heterocycloalkyl, aryl, or heteroaryl;
iv) amino acids, wherein the amino acid is linked via ester linkage at the point of attachment;
enantiomers, diastereomers, racemates, pharmaceutically acceptable salts or solvates thereof.

4. The compound of formula II, according to claim 3: 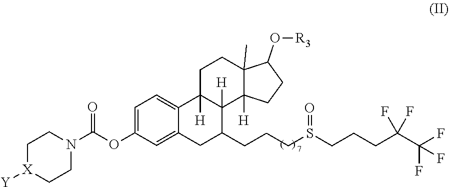
wherein X is selected from O, C, N; and
Y is selected from group comprising optionally substituted aryl, heteroaryl, heterocycloalkyl, or cycloalkyl; wherein the one or more substitution is selected from alkyl or alkoxy; and
R₃ is selected independently from group comprising of:
i) 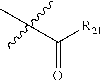
wherein R₂₁ is optionally substituted alkyl; wherein one or more substitution is selected from amino, phosphate, alkylphosphate, or hydroxy;
ii) 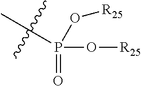
wherein each R₂₅ is selected independently from group comprising of hydrogen; optionally substituted alkyl;
iii) 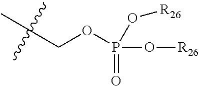
wherein each R₂₆ is selected independently from hydrogen; optionally substituted alkyl;
iv) amino acids; wherein the amino acid is linked via ester linkage at the point of attachment;
enantiomers, diastereomers, racemates, pharmaceutically acceptable salts or solvates thereof.

5. The compound of formula III, according to claim 1: 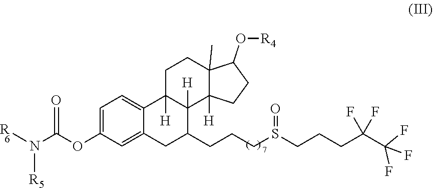
wherein R₅ is selected from hydrogen, alkyl;
R₆ is selected from group comprising optionally substituted alkyl, aryl, heteroaryl, heterocycloalkyl, or cycloalkyl; and
R₄ is selected independently from group comprising of:
i) 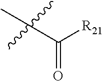
wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, hydroxy, alkylcarbonyl, formyl, halo, alkylphosphate, phosphate, cyano, nitro, alkoxy, amino, alkoxycarbonyl, alkenyl, alkynyl, alkylthio and arylcarbonyl;
ii) 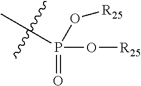
wherein each R₂₅ is selected independently from hydrogen; optionally substituted alkyl, aryl, or heteroaryl;
iii) 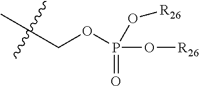
wherein each R₂₆ is selected independently from hydrogen; optionally substituted alkyl, cycloalkyl, heterocycloalkyl, aryl, or heteroaryl;
iv) amino acids, wherein the amino acid is linked via ester linkage at the point of attachment;
enantiomers, diastereomers, racemates, pharmaceutically acceptable salts or solvates thereof.

6. The compound of formula III, according to claim 5: 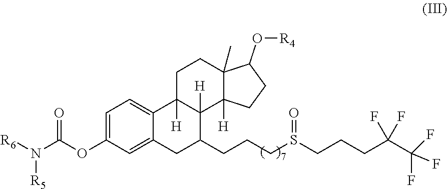
Wherein R₅ is selected from hydrogen, alkyl;
R₆ is selected from group comprising optionally substituted alkyl or aryl, wherein one or more substitution is selected from alkyl, alkoxy, halo, heteroaryl, heterocycloalkyl, cycloalkyl, amino, or hydroxy; and
R₄ is selected independently from group consisting of:
i) 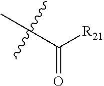
wherein R₂₁ is optionally substituted alkyl; wherein one or more substitution is selected from amino, phosphate, alkylphosphate, or hydroxy;
ii) 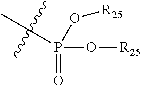
wherein each R₂₅ is selected independently from hydrogen, or optionally substituted alkyl;
iii) 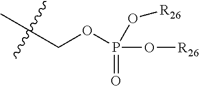
wherein each R₂₆ is selected independently from hydrogen or optionally substituted alkyl;
iv) amino acids, wherein the amino acid is linked via ester linkage at the point of attachment;
enantiomers, diastereomers, racemates, pharmaceutically acceptable salts or solvates thereof.

7. The compound according to claim 1, selected from group consisting of: 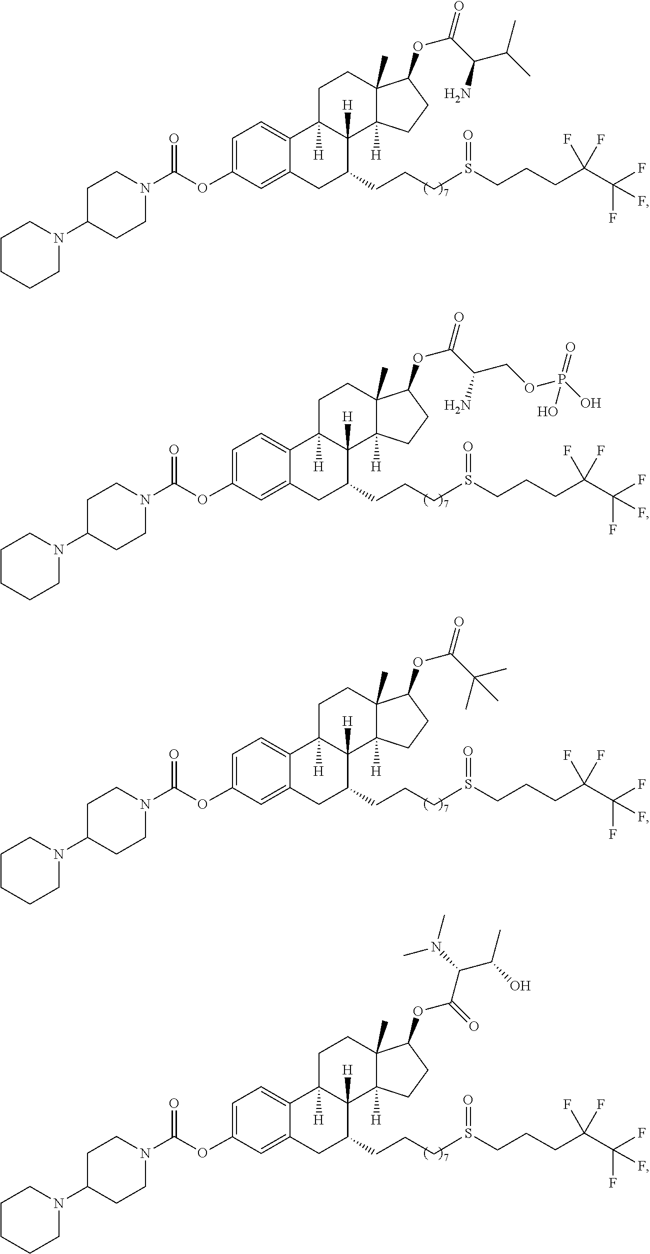 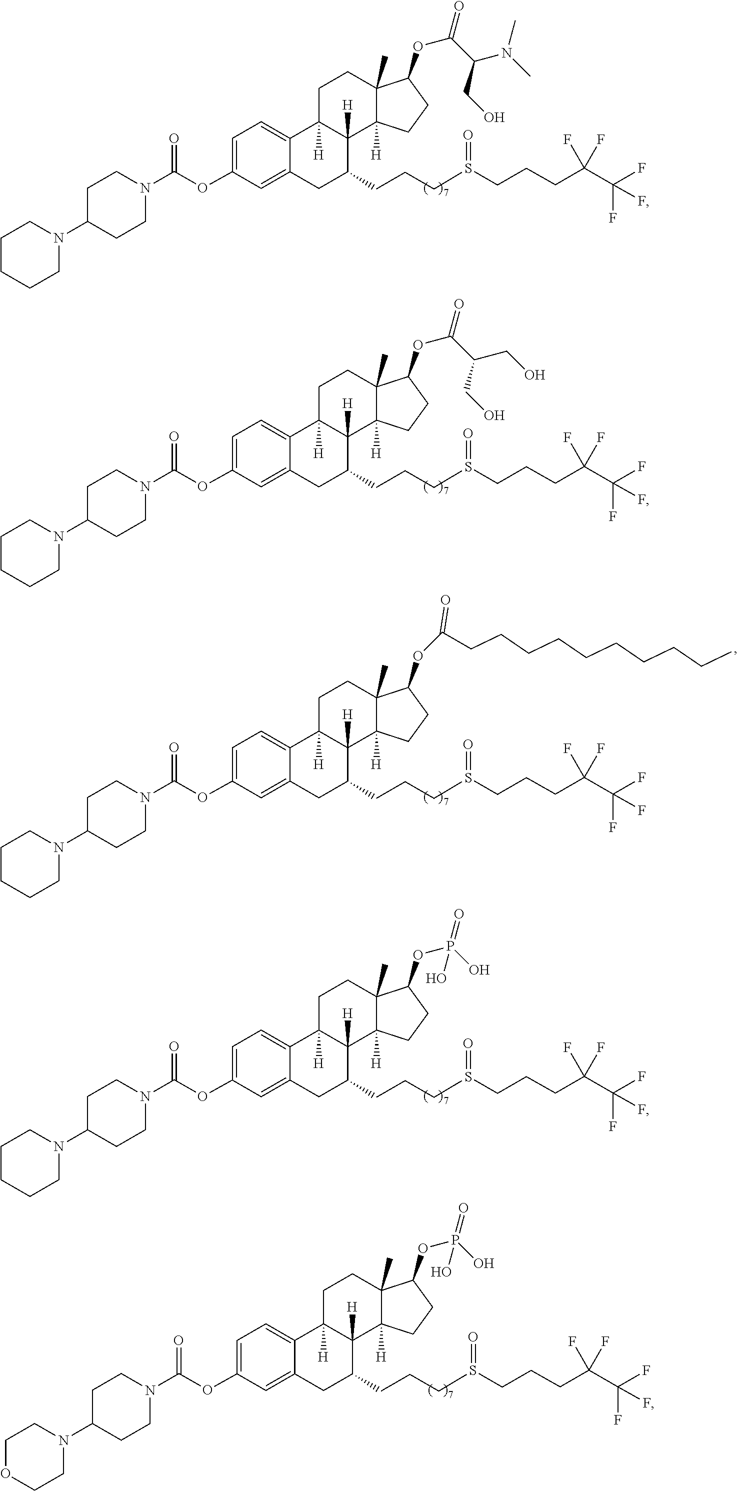 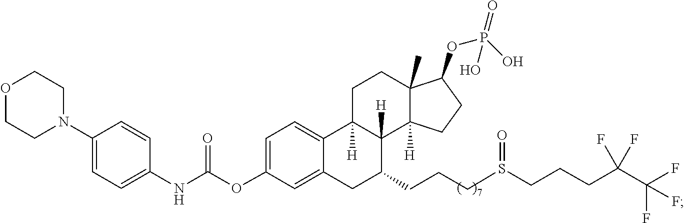
pharmaceutically acceptable salts or solvates thereof.

8. A compound of formula IV: 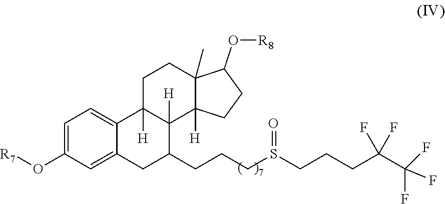
wherein R₇ is 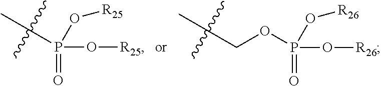
wherein each R₂₅ and R₂₆ is selected independently from hydrogen, optionally substituted alkyl, aryl or heteroaryl; and
R₈ is selected independently from group comprising of:
i) 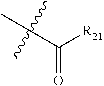
wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio and arylcarbonyl;
ii) 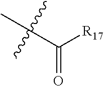
wherein R₁₇ is selected from NH₂, NHR₁₈ or NR₁₉R₂₀; R₁₈ is selected from group comprising optionally substituted alkyl, alkenyl, alkynyl, cycloalkyl, heterocycloalkyl, aryl, or heteroaryl; R₁₉ and R₂₀ are selected independently from group comprising optionally substituted alkyl, aryl, heteroaryl, heterocycloalkyl, cycloalkyl; or R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, oxo, cyano, nitro, amino, alkoxy, alkoxycarbonyl, —C(O)-aminoacid, carboxyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
iii) 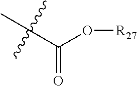
wherein R₂₇ is selected from group comprising optionally substituted alkyl, alkenyl, alkynyl, cycloalkyl, heterocycloalkyl, aryl, or heteroaryl;
iv) 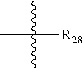
wherein R₂₈ is selected from group comprising optionally substituted alkyl, alkenyl, alkynyl, alkoxy, cycloalkyl, heterocycloalkyl, aryl or heteroaryl; wherein one or more substitution is selected from formyl, halo, phosphate, cyano, nitro, or amino;
v) amino acid, wherein the amino acid is linked via ester linkage at the point of attachment;
enantiomers, diastereomers, racemates, pharmaceutically acceptable salts or solvates thereof.

9. The compound of formula IV according to claim 8, wherein R₂₁ is selected from optionally substituted alkyl, alkenyl, alkynyl, cycloalkyl, aryl, heteroaryl; wherein one or more substitution is selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate.

10. The compound of formula V, according to claim 8: 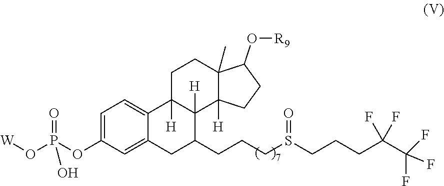
wherein W is selected from group comprising of hydrogen, or optionally substituted alkyl; and
R₉ is selected independently from group comprising of:
i) 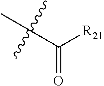
wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, aryl, heteroaryl;
ii) 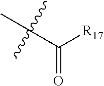
wherein R₁₇ is selected from NHR₁₈, or NR₁₉R₂₀; wherein R₁₈ is selected from group comprising optionally substituted alkyl, cycloalkyl, heterocycloalkyl, aryl and heteroaryl; R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the R₂₁ radicals are independently selected independently from the group consisting of optionally substituted alkyl, carboxyl, alkylcarbonyl, amino, —C(O)-aminoacid, cycloalkyl, aryl, aralkyl, heterocyclyoalkyl;
iii) 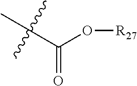
wherein R₂₇ is group comprising selected from optionally substituted alkyl, cycloalkyl, heterocycloalkyl, aryl, or heteroaryl; wherein the substitution can be amino;
iv) 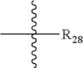
wherein R₂₈ is selected from group comprising optionally substituted alkyl, cycloalkyl, heterocycloalkyl, aryl or heteroaryl; wherein the substitution is amino;
v) amino acid, wherein the amino acid is linked via ester linkage at the point of attachment;
enantiomers, diastereomers, racemates, pharmaceutically acceptable salts or solvates thereof.

11. The compound of formula V-A, according to claim 8: 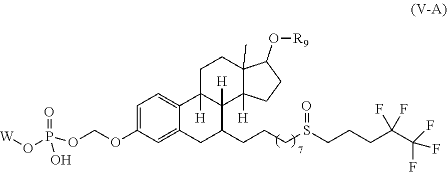
wherein W is selected from group comprising of hydrogen, or optionally substituted alkyl; and
R₉ is selected independently from group comprising of:
i) 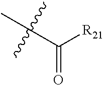
wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, aryl, heteroaryl;
ii) 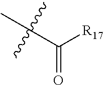
wherein R₁₇ is selected from NHR₁₈, or NR₁₉R₂₀; wherein R₁₈ is selected from group comprising optionally substituted alkyl, cycloalkyl, heterocycloalkyl, aryl and heteroaryl; R₁₉ and R₂₀ are taken together along with the nitrogen to which they are attached to form a three to seven membered saturated, partially unsaturated or unsaturated heterocyclic ring and may optionally be substituted at a substitutable position with one or more R₂₁ radicals, wherein the R₂₁ radicals are independently selected independently from the group consisting of optionally substituted alkyl, carboxyl, alkylcarbonyl, amino, —C(O)-aminoacid, cycloalkyl, aryl, aralkyl, heterocyclyoalkyl;
iii) 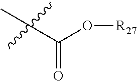
wherein R₂₇ is selected from group comprising optionally substituted alkyl, cycloalkyl, heterocycloalkyl, aryl, or heteroaryl; wherein the substitution can be amino;
iv) 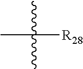
wherein R₂₈ is selected from group comprising optionally substituted alkyl, cycloalkyl, heterocycloalkyl, aryl or heteroaryl; wherein the substitution is amino;
v) amino acid, wherein the amino acid is linked via ester linkage at the point of attachment;
enantiomers, diastereomers, racemates, pharmaceutically acceptable salts or solvates thereof.

12. The compound according to claim 8, selected from group consisting of: 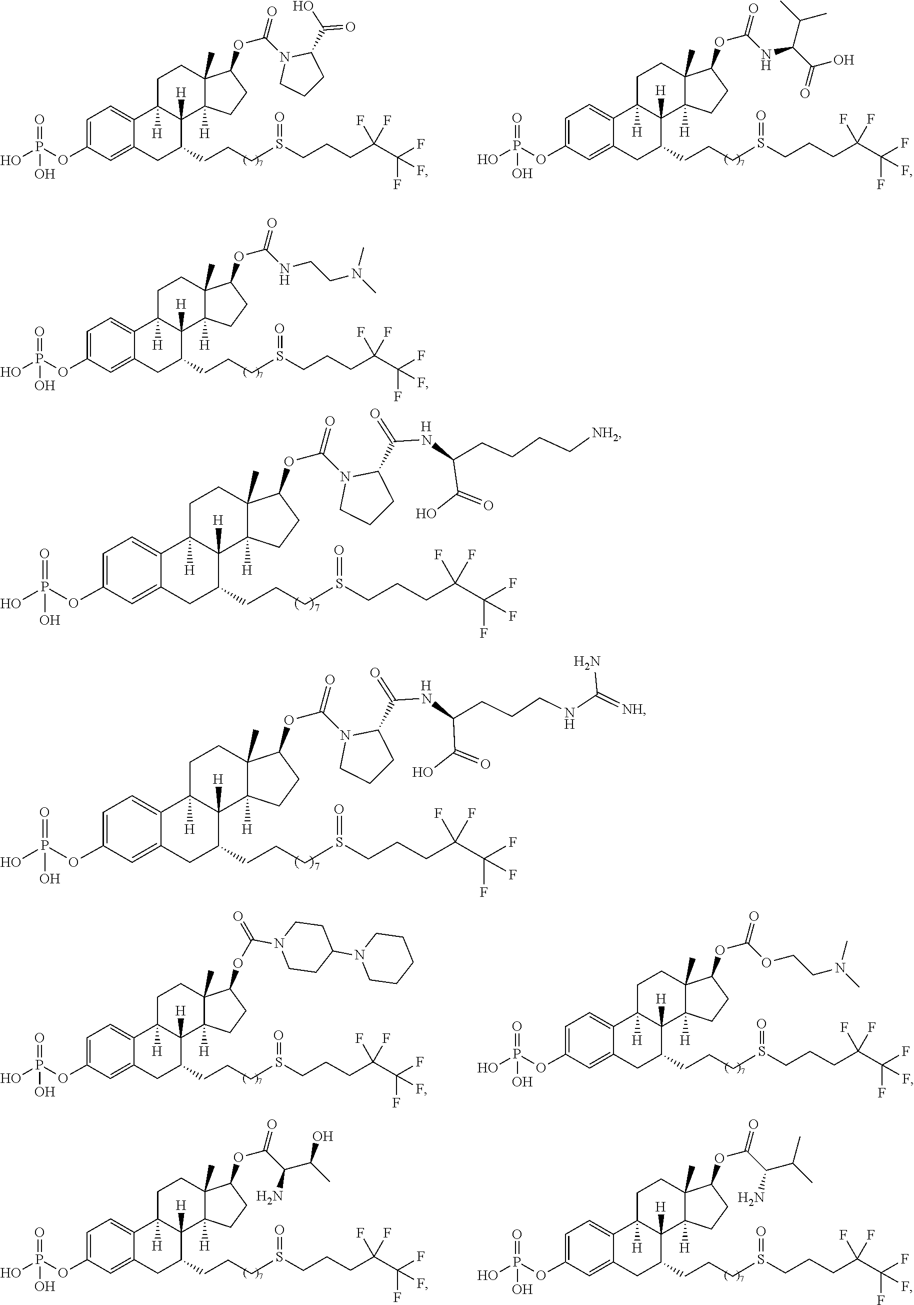 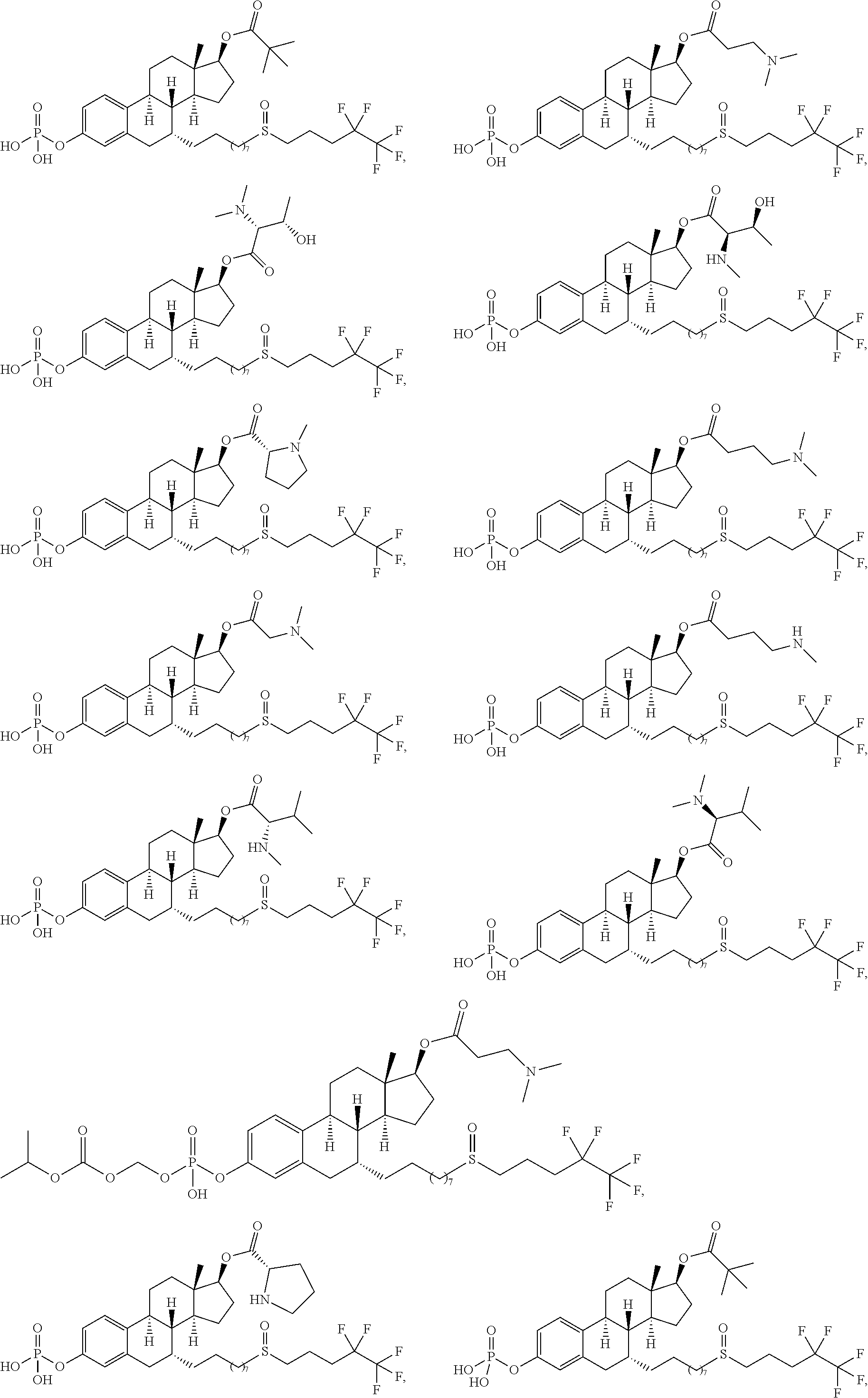 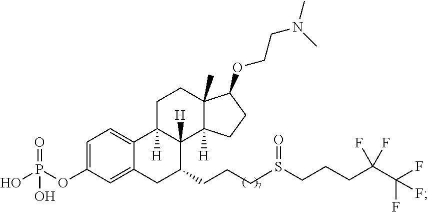
pharmaceutically acceptable salts or solvates thereof.

13. A compound of formula VI: 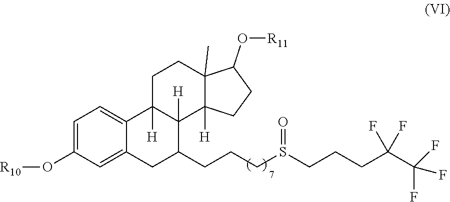
wherein, R₁₀ is 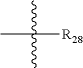
wherein R₂₈ is selected from group comprising optionally substituted alkyl, alkenyl, alkynyl, alkoxy, cycloalkyl, heterocycloalkyl, aryl, or heteroaryl; wherein one or more substitution is selected from formyl, halo, phosphate, cyano, nitro, amino, hydroxy, heterocycloalkyl, or alkoxy; and
R₁₁ is hydrogen or 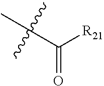
where R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, amino, alkoxycarbonyl, alkenyl, alkynyl, alkylthio, arylcarbonyl;
with the proviso that when R₁₁ is hydrogen, R₁₀ is not methyl or ethyl;
enantiomers, diastereomers, racemates, pharmaceutically acceptable salts or solvates thereof.

14. The compound of formula (VI) according to claim 13, R₂₁ is selected from optionally substituted alkyl, alkenyl, alkynyl, cycloalkyl, aryl, heteroaryl; wherein one or more substitution is selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate.

15. The compound of formula VII, according to claim 13: 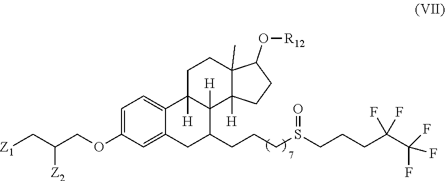
wherein, each Z₁ and Z₂ is independently selected from optionally substituted hydroxy, phosphate, or heterocycloalkyl; and
R₁₂ is hydrogen or 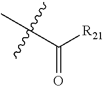
wherein R₂₁ is selected from group comprising optionally substituted alkyl, alkenyl, alkynyl, cycloalkyl, aryl, heteroaryl; or —CR₂₂R₂₃; where R₂₂ and R₂₃ are taken together along with the carbon to which they are attached to form a three to seven membered saturated, partially unsaturated or unsaturated heterocyclic ring in which up to 4 carbon atoms are replaced by heteroatoms chosen from the group consisting of O, S or N and may optionally be substituted at a substitutable position with one or more R₂₄ radicals, wherein one or more R₂₄ radicals are independently selected at each occurrence from the group consisting of optionally substituted alkyl, cycloalkyl, heterocycloalkyl, aryl, heteroaryl, alkylcarbonyl, formyl, halo, alkylphosphate, phosphate, cyano, nitro, alkoxy, alkoxycarbonyl, alkenyl, alkynyl, alkylthio, arylcarbonyl;
enantiomers, diastereomers, racemates, pharmaceutically acceptable salts or solvates thereof.

16. The compound of formula VII, according to claim 13: 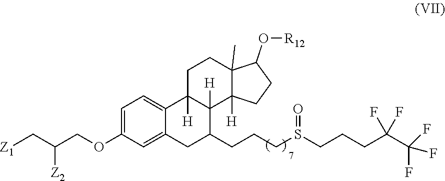
wherein, each Z₁ and Z₂ is independently selected from optionally substituted hydroxy, phosphate, or heterocycloalkyl; and
R₁₂ is hydrogen or 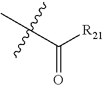
wherein R₂₁ is selected from group comprising optionally substituted alkyl, alkenyl, alkynyl, cycloalkyl, aryl, or heteroaryl; wherein one or more substitution is selected from alkyl, amino, or cycloalkyl;
enantiomers, diastereomers, racemates, pharmaceutically acceptable salts or solvates thereof.

17. The compound according to claim 13, selected from group consisting of: 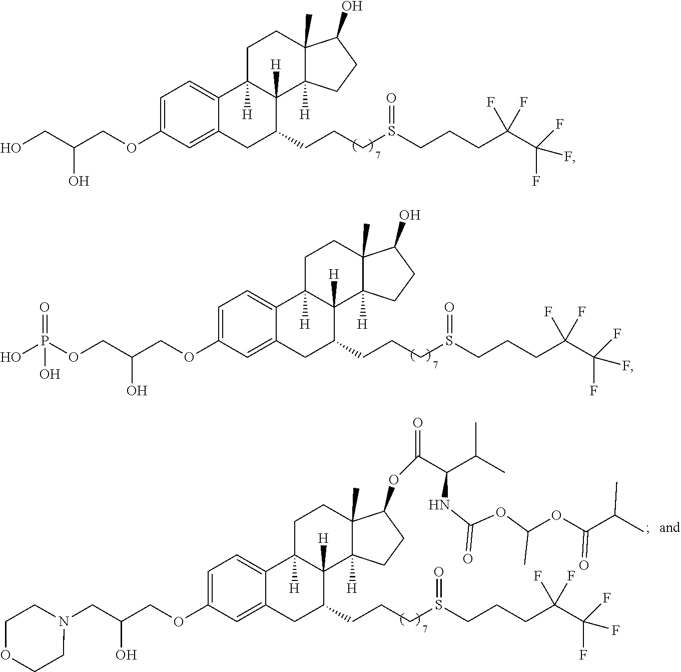
pharmaceutically acceptable salts or solvates thereof.

18. A compound of formula VIII: 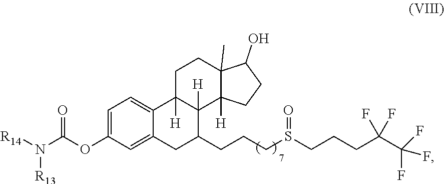
wherein R₁₃ are selected from group comprising H; optionally substituted alkyl, aryl, heteroaryl, heterocycloalkyl, cycloalkyl; and R₁₄ are selected from group comprising optionally substituted alkyl, aryl, heteroaryl, heterocycloalkyl, cycloalkyl; or R₁₃ and R₁₄ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of optionally substituted alkyl, alkylcarbonyl, formyl, halo, haloalkyl, alkylphosphate, phosphate, phosphonyl, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl;
enantiomers, diastereomers, racemates, pharmaceutically acceptable salts or solvates thereof.

19. The compound of formula VIII, according to claim 18: 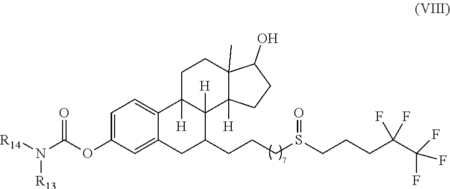
wherein R₁₃ and R₁₄ are taken together along with the nitrogen to which they are attached to form a three to seven membered saturated, partially unsaturated or unsaturated heterocyclic ring and may optionally be substituted at a substitutable position with one or more R₂₁ radicals, wherein the one or more R₂₁ radicals are independently selected at each occurrence from the group consisting of substituted alkyl, alkylcarbonyl, formyl, halo, haloalkyl, alkylphosphate, phosphate, phosphonyl, oxo, cyano, nitro, amino, alkoxy, alkoxycarbonyl, carboxyalkyl, hydroxyalkyl, alkenyl, alkynyl, alkylthio, cycloalkyl, aryl, heteroaryl, aralkyl, heterocycloalkyl, alkylthioalkyl, arylcarbonyl, aralkylcarbonyl, alkoxyalkyl; enantiomers, diastereomers, racemates, pharmaceutically acceptable salts or solvates thereof; wherein one or more substitution on radical R₂₁ is selected from group consisting of alkyl, alkenyl, alkynyl, cycloalkyl, heterocycloalkyl, aryl, heteroaryl, amino, alkylamino, acyl, acyloxy, acylamino, aminocarbonyl, alkoxycarbonyl, alkoxyalkyloxy, alkoxycarbonyloxy, carbamate, sulfinyl, sulfonyl, alkoxy, sulfanyl, halogen, carboxy, trihalomethyl, cyano, hydroxy, mercapto, nitro, phosphate, alkylphosphate.

20. The compound of formula VIII, according to claim 18: 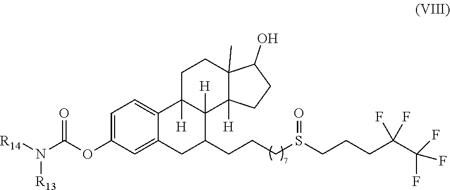
wherein R₁₃ is selected from hydrogen, alkyl; and R₁₄ is selected from group comprising optionally substituted alkyl, heterocycloalkyl, or aryl; wherein one or more substitution is selected from alkyl, alkoxy, halo, heteroaryl, heterocycloalkyl, cycloalkyl, amino, or hydroxy;
enantiomers, diastereomers, racemates, pharmaceutically acceptable salts or solvates thereof.

21. The compound according to claim 20, selected from group consisting of: 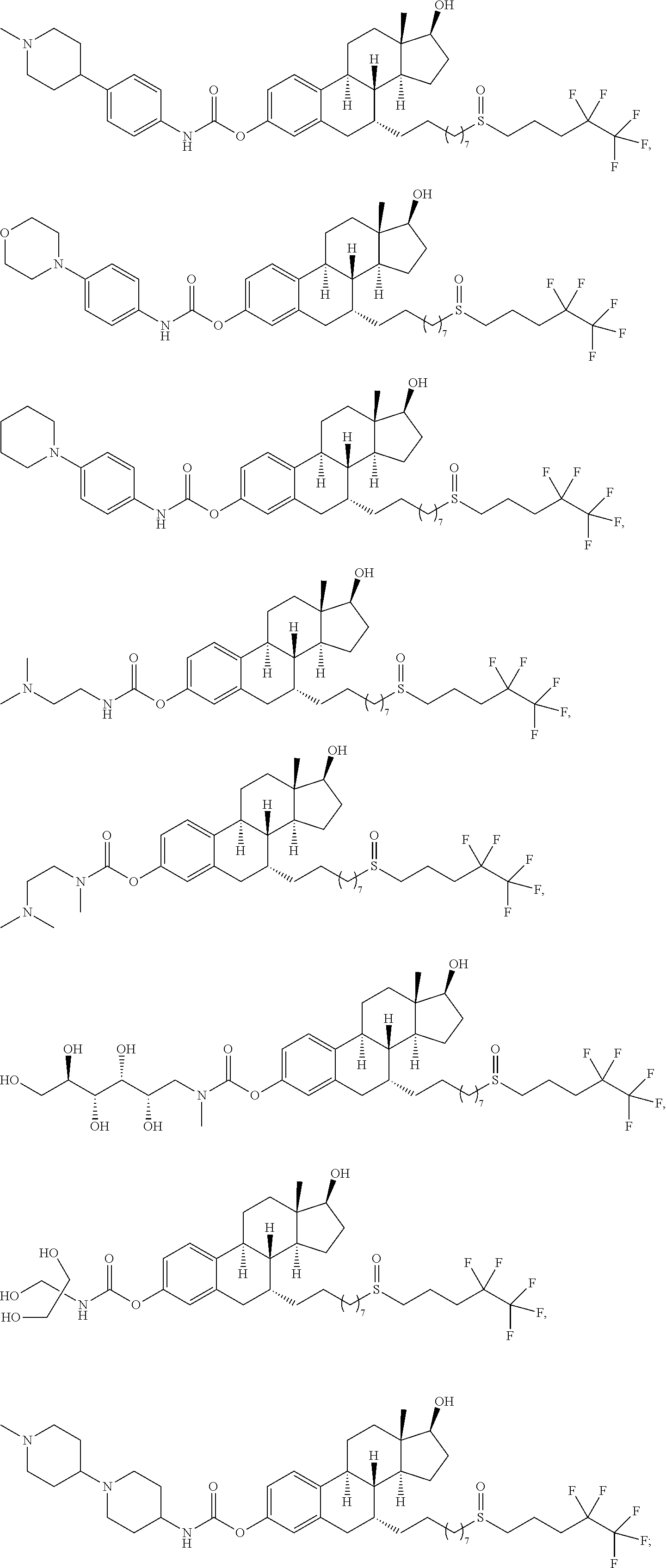
pharmaceutically acceptable salts or solvates thereof.

22. A compound of formula IX according to claim 18, 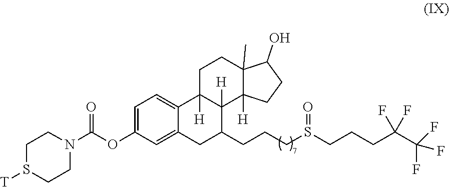
wherein S is selected from O, C, or N; and
T is selected from group comprising optionally substituted aryl, heteroaryl, heterocycloalkyl, cycloalkyl, amino, 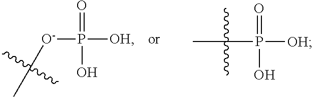
wherein one or more substitution is selected from alkyl, alkoxy;
enantiomers, diastereomers, racemates, pharmaceutically acceptable salts or solvates thereof.

23. The compound according to claim 22, selected from group consisting of: 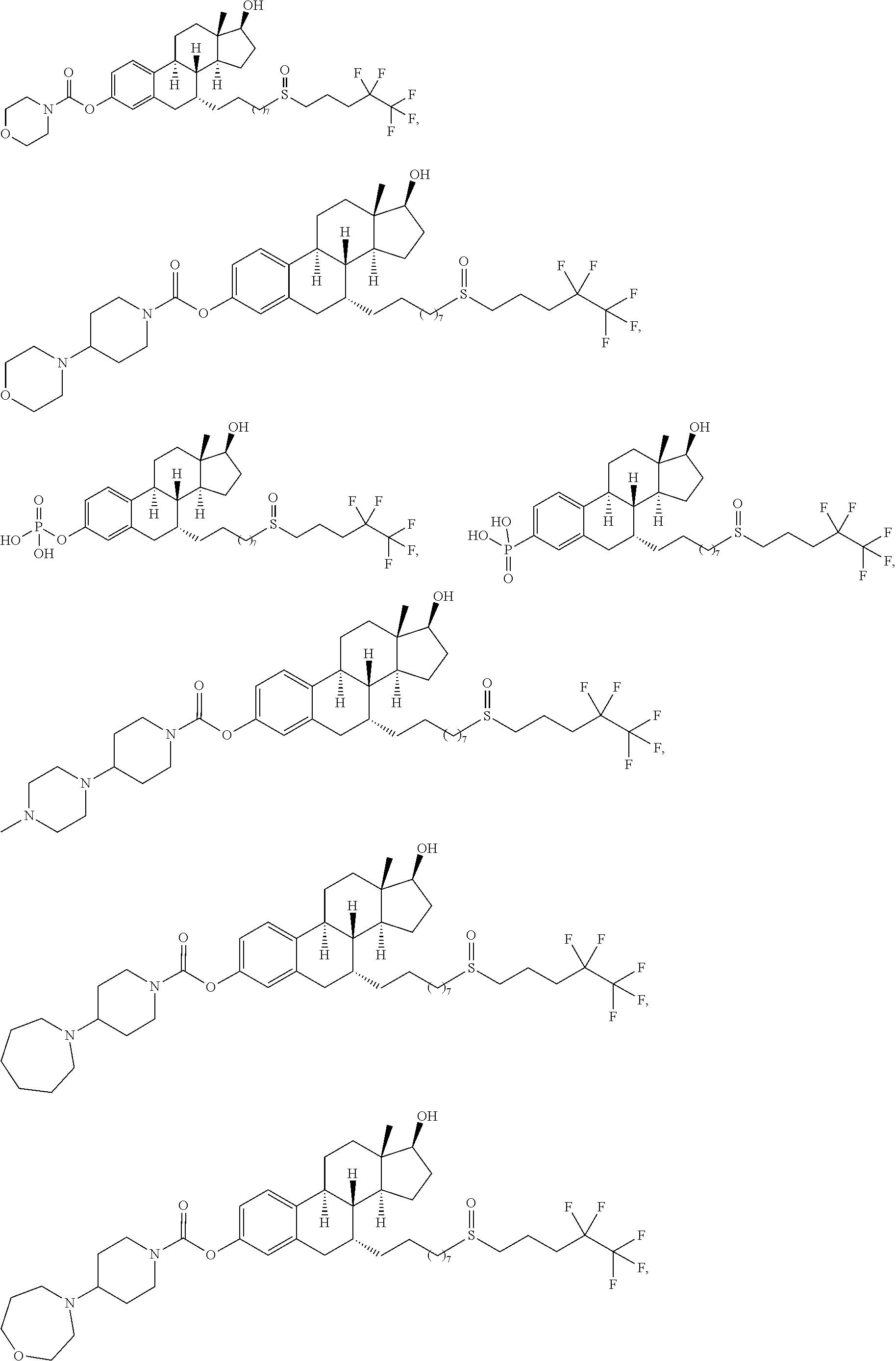 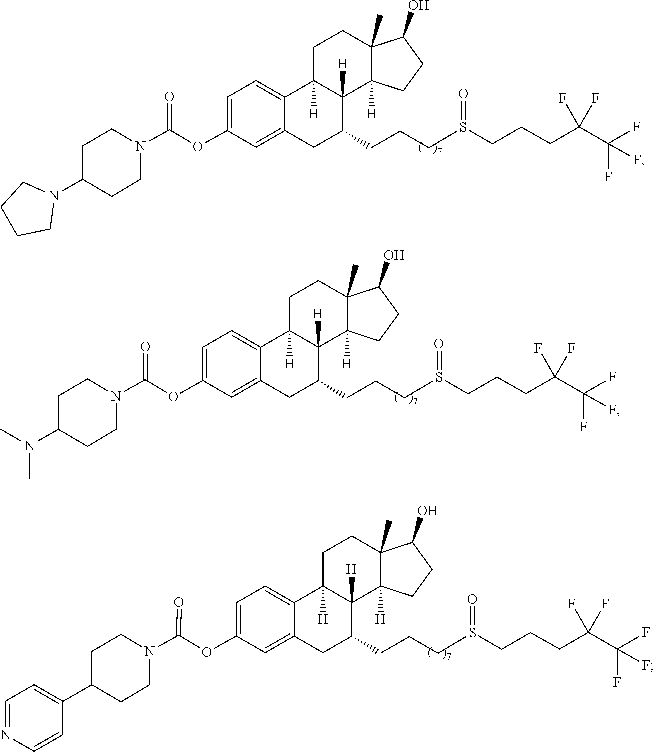
pharmaceutically acceptable salts or solvates thereof.

24. A compound of formula X: 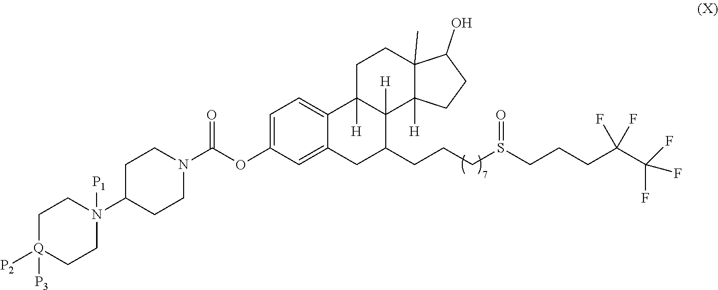
wherein, Q is selected from C or N; and
when Q is C, P₁ is selected independently from optionally substituted alkyl, optionally substituted 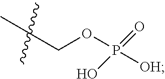 [SMILES: *COP(=O)(O)O]
wherein substitution on 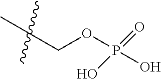 [SMILES: *COP(=O)(O)O]
is alkyl; and P₂ and P₃ is selected independently from hydrogen or alkyl; and
when Q is N, P₂ is selected independently from optionally substituted alkyl, optionally substituted 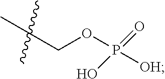 [SMILES: *COP(=O)(O)O]
wherein substitution on 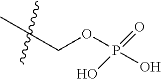 [SMILES: *COP(=O)(O)O]
is alkyl; P₁ is hydrogen and P₃ is optionally substituted alkyl;
enantiomers, diastereomers, racemates, pharmaceutically acceptable salts or solvates thereof.

25. The compound according to claim 24, selected from group consisting of: 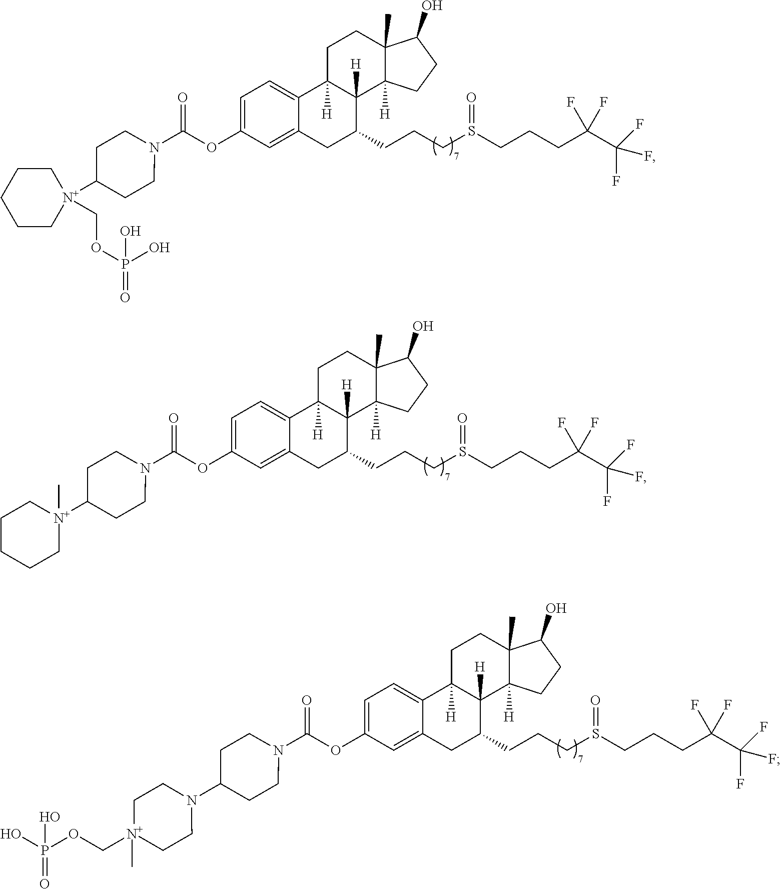
pharmaceutically acceptable salts or solvates thereof.

26. A compound of formula XI: 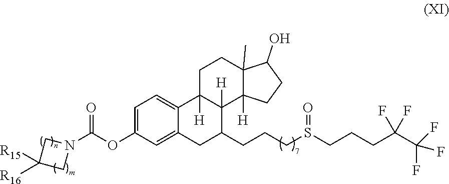
wherein R₁₅ and R₁₆ together forms a cyclic structure; wherein the said cyclic structure is selected from optionally substituted cycloalkyl, or heterocycloalkyl; wherein one or more substitution is selected from alkyl, alkoxy, halo, amino, or hydroxy; m and n can be independently 1 to 3;
enantiomers, diastereomers, racemates, pharmaceutically acceptable salts or solvates thereof.

27. The compound according to claim 26, selected from group consisting of: 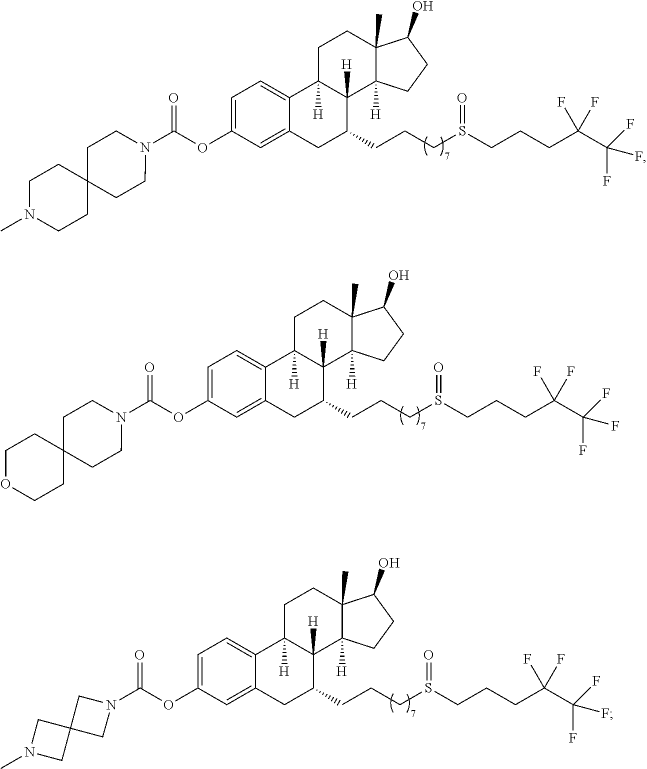
pharmaceutically acceptable salts, or solvates thereof.

28. The compound selected from group consisting of: 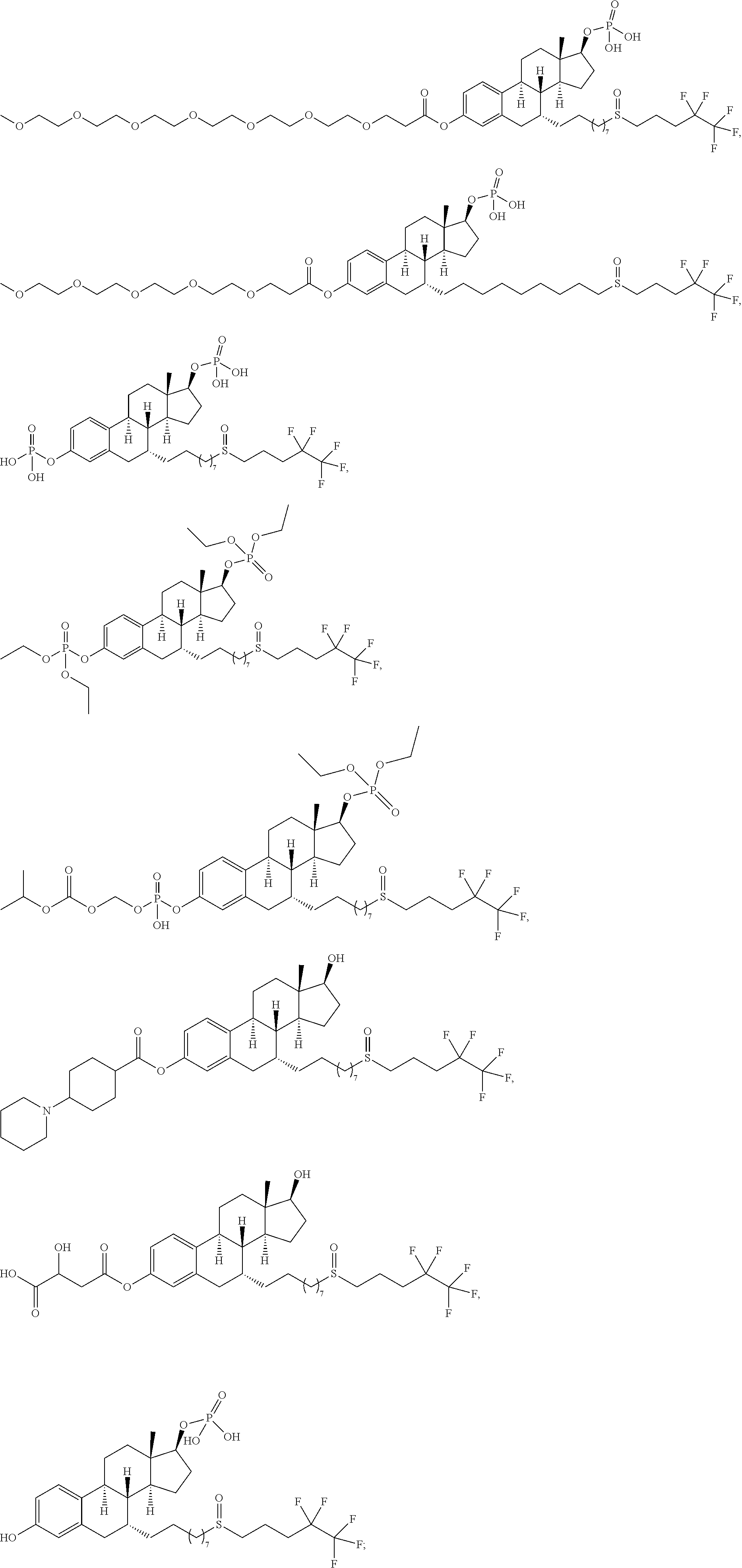
pharmaceutically acceptable salts or solvates thereof.

29. A compound of formula

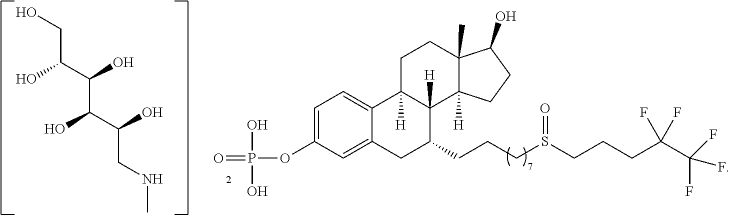

30. A pharmaceutical composition comprising a compound selected from the group consisting of formula I, formula II, formula III, formula IV, formula V, formula VI, formula VII, formula VIII, formula IX, formula X and formula XI; enantiomers, diastereomers, racemates, pharmaceutically acceptable salts or solvates thereof; and a pharmaceutical acceptable excipient.

31. A method of treating breast cancer comprising administration of a compound selected from group consisting of formula I, formula II, formula III, formula IV, formula V, formula V-A, formula VI, formula VII, formula VIII, formula IX, formula X, formula XI; enantiomers, diastereomers, racemates, pharmaceutically acceptable salts or solvates thereof.
